(19) United States
(12) Patent Application Publication
(10) Pub. No.: US 2024/0299412 A1
(43) Pub. Date: Sep. 12, 2024

(54) DRUG FORMULATIONS OF 4-(3,3-DIFLUORO-2,2-DIMETHYL-PROPANOYL) -3,5-DIHYDRO-2H-PYRIDO[3,4-f][1,4]OXAZEPINE-9-CARBONITRILE

(71) Applicant: Genzyme Corporation, Cambridge, MA (US)

(72) Inventors: Jerome Arigon, Paris (FR); Marilyn Calderone, Paris (FR); Brigitte Illel, Paris (FR)

(73) Assignee: Genzyme Corporation, Cambridge, MA (US)

(21) Appl. No.: 18/659,600

(22) Filed: May 9, 2024

Related U.S. Application Data
(63) Continuation of application No. PCT/US2024/012330, filed on Jan. 22, 2024.
(60) Provisional application No. 63/440,636, filed on Jan. 23, 2023.
(60) Provisional application No. 63/614,973, filed on Dec. 27, 2023.

(51) Int. Cl. A61K 31/553; A61K 9/20; A61K 9/28; A61K 9/48

(52) U.S. Cl. CPC A61K 31/553; A61K 9/2009; A61K 9/2018; A61K 9/2054; A61K 9/2059; A61K 9/2893; A61K 9/4825

(57) ABSTRACT

This disclosure relates to the field of therapeutic tyrosine kinase inhibitors, in particular receptor-interacting serine/threonine-protein kinase 1 (RIPK1) inhibitors. Solid and liquid formulations of 4-(3,3-difluoro-2,2-dimethyl-propanoyl)-3,5-dihydro-2H-pyrido[3,4-f][1,4]oxazepine-9-carbonitrile are described.

### CROSS-REFERENCE TO RELATED APPLICATIONS

This application claims the benefit of priority to U.S. Provisional Application No. 63/440,636, filed Jan. 23, 2023, and U.S. Provisional Application No. 63/614,973, filed Dec. 27, 2023, the contents of each of which are incorporated by reference herein in their entirety.

### FIELD

The present disclosure relates to pharmaceutical formulations of 4-(3,3-difluoro-2,2-dimethyl-propanoyl)-3,5-dihydro-2H-pyrido[3,4-f][1,4]oxazepine-9-carbonitrile, the process of preparing the formulations, and methods of use thereof.

### BACKGROUND AND SUMMARY

Receptor-interacting protein kinase 1 (RIPK1) is an intracellular protein involved in the regulation of inflammation, cytokine release, and cell death. RIPK1 is activated in response to several inflammatory stimuli, most notably tumor necrosis factor alpha (TNF-α) signaling through its receptor 1 (TNF1), with subsequent RIPK1 initiation of a complex signaling cascade that triggers intracellular responses, including cytokine release, microglial activation, and necroptosis, a regulated form of cell death. Inhibition of RIPK1 activity has been shown to protect against necroptotic cell death in vitro across a range of cell death models. Similarly, in various animal models of diseases ranging from Alzheimer's disease (AD) to amyotrophic lateral sclerosis (ALS), inhibition of RIPK1 protects against their respective pathologies and attributed cell death. These nonclinical findings, coupled with observations of increased RIPK1 activity in human diseases, suggest that inhibition of RIPK1 could ameliorate ALS.

The RIPK1 pathway is activated in spinal cords from patients with ALS. These postmortem samples show elevated levels of RIPK1 and its downstream signaling partners, receptor-interacting serine/threonine-protein kinase 3 (RIPK3) and mixed lineage kinase domain-like pseudokinase (MLKL). Similar increases have been observed in the spinal cord of the SOD1 (G93A) transgenic mouse with ALS. Furthermore, both genetic inhibition of the RIPK1 pathway (RIPK3−/−) and treatment with a RIPK1 inhibitor reduced axonal pathology and delayed the onset of motor dysfunction in this model. Several ALS-inducing mutations, including TANK-binding kinase 1 (TBK1) and optineurin (OPTN), have been shown to sensitize cells to RIPK1-dependent cell death and inflammation. Notably, another RIPK1 activity inhibitor, transforming growth factor α-activated kinase 1 (TAK1), declines with age, which may predispose the CNS to neuroinflammation and neurodegeneration in the setting of genetic or environmental stresses. In summary, both preclinical and patient-derived data have suggested RIPK1 may be a key mediator of necroptosis and inflammatory pathways in ALS.

RIPK1 has an important role in modulating inflammatory responses mediated by nuclear-factor kappa-light chain enhancer of activated B cells (NF-kB). More recent research has shown that its kinase activity controls necroptosis, a form of necrotic cell death. Further, RIPK1 is part of a pro-apoptotic complex indicating its activity in regulating apoptosis. Dysregulation of receptor-interacting protein kinase 1 signaling can lead to excessive inflammation or cell death. Research suggests that inhibition of RIPK1 is a potential clinical target for diseases involving inflammation or cell death. RIPK1 kinase has emerged as a promising therapeutic target for the treatment of a wide range of human neurodegenerative, autoimmune, and inflammatory diseases.

The compound 4-(3,3-difluoro-2,2-dimethyl-propanoyl)-3,5-dihydro-2H-pyrido[3,4-f][1,4]oxazepine-9-carbonitrile, (hereinafter also referred to as “Compound 1”), depicted below, is a RIPK1 inhibitor:

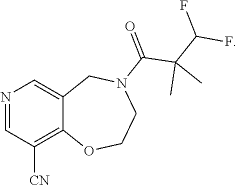 [SMILES: CC(C)(C(=O)N1CCOc2c(C#N)cncc2C1)C(F)F]

One factor in assessing the suitability of a compound as a therapeutic agent is whether the compound as a therapeutic agent can be administered in a form that is easily absorbed by the body and also shelf-stable. The pharmaceutically active substance used to prepare the treatment should be as pure as possible and its stability on long-term storage should be guaranteed under various environmental conditions. These properties are useful to prevent the appearance of unintended degradation products in pharmaceutical compositions, which degradation products may be potentially toxic or result simply in reducing the potency and/or efficacy of the composition.

A primary concern for the large-scale manufacture of pharmaceutical compounds is that the active substance should have a stable crystalline morphology to ensure consistent processing parameters and pharmaceutical quality. If an unstable crystalline form is used, crystal morphology may change during manufacture and/or storage, resulting in quality control problems and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” Although polymorphs of a given substance have the same chemical composition, they may differ from each other with respect to one or more physical properties, such as solubility, dissociation, true density, dissolution, melting point, crystal shape, morphology, particle size, compaction behavior, flow properties, and/or solid-state stability.

Although it is known that the preparation of crystalline forms may improve the physical or pharmaceutical properties of a pharmaceutically active compound, it is not possible to predict whether a compound exists in crystalline form(s) or which crystalline form(s) may possess advantages for a particular purpose prior to the actual preparation and characterization of the crystalline form. In particular, such advantages, in a non-limiting manner could include better processability, solubility or shelf-life stability, just to name a few. Other advantages may also include biological properties such as improved bioavailability, reduced adverse reactions at the GI tract (for example irritation of the GI tract, partial degradation of the compound, etc.), or better deliverability of the drug to the intended target site among other advantages.

Physical properties of a drug substance present a set of challenges that can influence selection of excipients and the manufacturing process. It is not possible to predict how certain excipients might impact the physical properties of a pharmaceutical formulation, such as a tablet, or affect the processibility of the pharmaceutical formulation. Certain combinations of excipients with the drug substance can impart physical and biological advantages over other combinations. In particular, such advantages may include improved linearity of tablet hardness and ejection force versus compression force. Tablet hardness can be an issue depending on the patient population. Extremely hard tablets may indicate that the binding force between the active ingredient and the excipient is too great, which may prevent the proper disintegration and dissolution of the tablet needed for an accurate dosage. Similarly, softer tablets may be the result of weak binding and may cause process issues during tablet film coating or packaging. Poor hardness tablet may also lead to tablet breakage during deblisterisation and impact patient compliance or the dose administered.

The present disclosure relates to a tablet comprising 4-(3,3-difluoro-2,2-dimethyl-propanoyl)-3,5-dihydro-2H-pyrido[3,4-f][1,4]oxazepine-9-carbonitrile (Compound 1) and at least one pharmaceutically acceptable excipient.

### BRIEF DESCRIPTION OF DRAWINGS

FIG. 1 shows an X-ray powder diffractogram of crystalline Form A of 4-(3,3-difluoro-2,2-dimethyl-propanoyl)-3,5-dihydro-2H-pyrido[3,4-f][1,4]oxazepine-9-carbonitrile.

FIG. 2 shows a Differential Scanning calorimetry/Thermal Gravimetric Analysis (DSC/TGA) thermogram of crystalline Form A of 4-(3,3-difluoro-2,2-dimethyl-propanoyl)-3,5-dihydro-2H-pyrido[3,4-f][1,4]oxazepine-9-carbonitrile.

FIG. 3 shows a polarized light microscopy image of crystalline Form A of 4-(3,3-difluoro-2,2-dimethyl-propanoyl)-3,5-dihydro-2H-pyrido[3,4-f][1,4]oxazepine-9-carbonitrile.

FIG. 4 shows a dynamic vapor sorption isotherm plot of crystalline Form A of 4-(3,3-difluoro-2,2-dimethyl-propanoyl)-3,5-dihydro-2H-pyrido[3,4-f][1,4]oxazepine-9-carbonitrile.

FIG. 5 shows an overlay of X-ray powder diffractograms of crystalline Form A of 4-(3,3-difluoro-2,2-dimethyl-propanoyl)-3,5-dihydro-2H-pyrido[3,4-f][1,4]oxazepine-9-carbonitrile before and after dynamic vapor sorption.

FIG. 6 shows an HPLC chromatogram of crystalline Form A of 4-(3,3-difluoro-2,2-dimethyl-propanoyl)-3,5-dihydro-2H-pyrido[3,4-f][1,4]oxazepine-9-carbonitrile.

FIGS. 7A and 7B provide Yasuda-Shedlovsky plots of pKa measurements for crystalline Form A of 4-(3,3-difluoro-2,2-dimethyl-propanoyl)-3,5-dihydro-2H-pyrido[3,4-f][1,4]oxazepine-9-carbonitrile.

FIG. 8 shows a polarized light microscopy image of a single crystal of crystalline Form A of 4-(3,3-difluoro-2,2-dimethyl-propanoyl)-3,5-dihydro-2H-pyrido[3,4-f][1,4]oxazepine-9-carbonitrile.

FIG. 9 shows an asymmetrical unit representation of crystalline Form A of 4-(3,3-difluoro-2,2-dimethyl-propanoyl)-3,5-dihydro-2H-pyrido[3,4-f][1,4]oxazepine-9-carbonitrile.

FIG. 10 shows a thermal ellipsoid (ORTEP) representation of crystalline Form A of 4-(3,3-difluoro-2,2-dimethyl-propanoyl)-3,5-dihydro-2H-pyrido[3,4-f][1,4]oxazepine-9-carbonitrile.

FIG. 11 shows the predicted chemical structure of Compound (1) as determined by single crystal analysis.

FIG. 12 shows a unit cell of crystalline Form A of 4-(3,3-difluoro-2,2-dimethyl-propanoyl)-3,5-dihydro-2H-pyrido[3,4-f][1,4]oxazepine-9-carbonitrile.

FIG. 13 shows a packing diagram of crystalline Form A of 4-(3,3-difluoro-2,2-dimethyl-propanoyl)-3,5-dihydro-2H-pyrido[3,4-f][1,4]oxazepine-9-carbonitrile shown along the a-axis.

FIG. 14 shows a packing diagram of crystalline Form A of 4-(3,3-difluoro-2,2-dimethyl-propanoyl)-3,5-dihydro-2H-pyrido[3,4-f][1,4]oxazepine-9-carbonitrile shown along the b-axis.

FIG. 15 shows a packing diagram of crystalline Form A of 4-(3,3-difluoro-2,2-dimethyl-propanoyl)-3,5-dihydro-2H-pyrido[3,4-f][1,4]oxazepine-9-carbonitrile shown along the c-axis.

FIG. 16 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile starting material, experimental single crystal, and calculated single crystal.

FIG. 17 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile starting material, and after 1 week of storage under the following conditions: 40° C./75% RH, 25° C./60% RH, 60° C.

FIG. 18 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile starting material, and after 4 weeks of storage under the following conditions: 40° C./75% RH, 25° C./60% RH, 60° C.

FIG. 19 shows the kinetic solubility curves of crystalline Form A of 4-(3,3-difluoro-2,2-dimethyl-propanoyl)-3,5-dihydro-2H-pyrido[3,4-f][1,4]oxazepine-9-carbonitrile in various biorelevant media at 37° C.

FIG. 20 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile before and after various solubility tests at 37° C.

FIG. 21 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile before and after various solubility tests at RT.

FIG. 22 shows an overlay of X-ray powder diffractograms comparing crystalline Form A of 4-(3,3-difluoro-2,2-dimethyl-propanoyl)-3,5-dihydro-2H-pyrido[3,4-f][1,4]oxazepine-9-carbonitrile before and after various pH solubility tests.

FIG. 23 shows an LC chromatogram and mass spectra of crystalline Form A of 4-(3,3-difluoro-2,2-dimethyl-propanoyl)-3,5-dihydro-2H-pyrido[3,4-f][1,4]oxazepine-9-carbonitrile after 24 hrs in pH 2.0.

FIG. 24 shows an LC chromatogram and mass spectra of crystalline Form A of 4-(3,3-difluoro-2,2-dimethyl-propanoyl)-3,5-dihydro-2H-pyrido[3,4-f][1,4]oxazepine-9-carbonitrile after 24 hrs in pH 8.0.

FIG. 25 shows an LC chromatogram and mass spectra of crystalline Form A of 4-(3,3-difluoro-2,2-dimethyl-propanoyl)-3,5-dihydro-2H-pyrido[3,4-f][1,4]oxazepine-9-carbonitrile after 96 hrs in pH 8.0.

FIG. 26A shows the free energy landscape at 298.15 K from step 4 of the calculations as discussed in Example 5, and FIG. 26B lists properties of the 30 most stable predicted crustal structures.

FIG. 27 shows an overlay of the molecular conformations in rank 1 (middle structure), rank 5 (top structure), and rank 6 (lower structure), with hydrogen atoms omitted for clarity. The diagram shows the molecular flexibility of −(3,3-difluoro-2,2-dimethyl-propanoyl)-3,5-dihydro-2H-pyrido[3,4-f][1,4]oxazepine-9-carbonitrile.

FIG. 28 shows a similarity matrix of the 30 most stable predicted structures, with values from 0.8 to 1.0 highlighted on a white-grey color scale.

FIG. 29 shows an overlay of the molecular conformations of rank 1 (white), rank 2 (crosshatch), and rank 3 (black). The structures only overlay in projection, not in three dimensions.

FIG. 30 shows an overlay of the single crystal structure of Form A (white) with rank 1 (black).

FIG. 31 shows the free energy landscape with the experimental forms indicated.

FIG. 32 shows the free energy landscape as a function of temperature.

FIG. 33 shows the XRPD spectrum of an amorphous form of 4-(3,3-difluoro-2,2-dimethyl-propanoyl)-3,5-dihydro-2H-pyrido[3,4-f][1,4]oxazepine-9-carbonitrile as described herein.

FIG. 34 shows a Differential Scanning calorimetry (DSC) thermogram of amorphous 4-(3,3-difluoro-2,2-dimethyl-propanoyl)-3,5-dihydro-2H-pyrido[3,4-f][1,4]oxazepine-9-carbonitrile.

FIG. 35 shows the XRPD spectrum of a substantially amorphous form of 4-(3,3-difluoro-2,2-dimethyl-propanoyl)-3,5-dihydro-2H-pyrido[3,4-f][1,4]oxazepine-9-carbonitrile as described herein.

FIG. 36 shows the XRPD spectrum of the substantially amorphous form of −(3,3-difluoro-2,2-dimethyl-propanoyl)-3,5-dihydro-2H-pyrido[3,4-f][1,4]oxazepine-9-carbonitrile shown in FIG. 35 after conversion to a crystalline form as described herein.

FIG. 37 shows a dissolution profile of a tablet of Formulation B5 according to Example 12 containing 20 mg of Compound 1.

FIG. 38 shows tablet harness and ejection force versus compression force for two different tablet formulations.

FIG. 39 shows the friability tests for two different tablet formulations.

FIG. 40 shows drug stability data for a tablet of Formulation B1 according to Example 12 containing 10 mg of Compound 1.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying figures, which are incorporated in and constitute a part of this specification, illustrate several embodiments and together with the description, serve to explain the principles described herein.

### DETAILED DESCRIPTION

Reference will now be made in detail to certain embodiments, examples of which are illustrated in the accompanying drawings. While the disclosure provides illustrated embodiments, it will be understood that they are not intended to limit the disclosure to those embodiments. On the contrary, the disclosure is intended to cover all alternatives, modifications, and equivalents, which may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless otherwise stated, the following terms used in the specification and claims are defined for the purposes of this disclosure and have the following meaning:

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “article of manufacture” and “kit” are used as synonyms.

As used herein, “the RIPK1 inhibitor,” “the RIPK1 inhibitor compound,” “Compound 1,” and “the compound”, refers to 4-(3,3-difluoro-2,2-dimethyl-propanoyl)-3,5-dihydro-2H-pyrido[3,4-f][1,4]oxazepine-9-carbonitrile having the following structure:

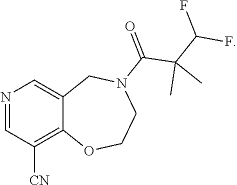 [SMILES: CC(C)(C(=O)N1CCOc2c(C#N)cncc2C1)C(F)F]

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Pharmaceutically acceptable excipient” includes, but is not limited to, diluents, disintegrants, glidants, lubricants, antifoaming agents, antioxidants, preservatives, dispersing agents and/or viscosity modulating agents, erosion facilitators, filling agents, flavoring agents, solubilizers, suspending agents, surfactants, and wetting agents.

As used herein, the term “crystalline” or “crystalline solid form,” refers to a solid form which is substantially free of any amorphous solid-state form.

In some embodiments, “substantially free” means less than about 10% w/w, less than about 9% w/w, less than about 8% w/w, less than about 7% w/w, less than about 6% w/w, less than about 5% w/w, less than about 4% w/w, less than about 3% w/w, less than about 2.5% w/w, less than about 2% w/w, less than about 1.5% w/w, less than about 1% w/w, less than about 0.75% w/w, less than about 0.50% w/w, less than about 0.25% w/w, less than about 0.10% w/w, or less than about 0.05% w/w of other crystalline forms of the compound and the amorphous compound. In some embodiments, “substantially free” means an undetectable amount of other crystalline forms of the compound and the amorphous compound.

As used herein, the term “substantially pure” or “substantially crystalline” means that the crystalline form contains at least 90 percent, for example at least 95 percent, such as at least 97 percent, and even at least 99 percent by weight of the indicated crystalline form compared to the total weight of the compound of all forms.

Alternatively, it will be understood that “substantially pure” or “substantially crystalline” means that the crystalline form contains less than 10 percent, for example less than 5 percent, such as less than 3 percent, and even less than 1 percent by weight of impurities, including other polymorphic, solvated or amorphous forms compared to the total weight of the compound of all forms.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. For example, an amorphous material is a solid material having no sharp characteristic signal(s) in its X-ray power diffractogram (i.e., is not crystalline as determined by XRPD). Instead, one or more broad peaks (e.g., halos) appear in its diffractogram. Broad peaks are characteristic of an amorphous solid.

As used herein, the term “substantially amorphous” refers to a solid material having little or no long-range order in the position of its molecules. For example, substantially amorphous materials have less than 15% crystallinity (e.g., less than 10% crystallinity or less than 5% crystallinity). “Substantially amorphous” includes the descriptor “amorphous,” which refers to materials having no (0%) crystallinity.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the RIPK1 inhibitor compound, that, when administered to a mammal for treating a disease, is sufficient to effect such treatment for the disease. The “therapeutically effective amount” will vary depending on the compound, the disease and its severity and the age, weight, etc., of the mammal to be treated.

An “XRPD pattern” or “X-ray powder diffraction pattern” is an x-y graph with diffraction angle (i.e., 2 θ) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns; this variability is usually greater than the variability in diffraction angles. Diffraction angle variability may also be sensitive to sample preparation. Other sources of variability come from instrument parameters and processing of the raw X-ray data: different X-ray instruments operate using different parameters and these may lead to slightly different XRPD patterns from the same solid form, and similarly different software packages process X-ray data differently and this also leads to variability. These and other sources of variability are known to those of ordinary skill in the pharmaceutical arts. Due to such sources of variability, it is usual to assign a variability of about ±0.2° θ to diffraction angles in XRPD patterns.

Before describing the present teachings in detail, it is to be understood that the disclosure is not limited to specific compositions or process steps, as such may vary.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

The present disclosure relates to a tablet comprising 4-(3,3-difluoro-2,2-dimethyl-propanoyl)-3,5-dihydro-2H-pyrido[3,4-f][1,4]oxazepine-9-carbonitrile (Compound 1) and at least one pharmaceutically acceptable excipient.

In some embodiments provided herein, the form of Compound 1 used to prepare the tablet is crystalline.

In some embodiments provided herein, the form of Compound 1 used to prepare the tablet is amorphous.

In some embodiments provided herein, the form of Compound 1 used to prepare the tablet is crystalline Form A (also referred to herein as Type A).

In some embodiments, the crystallinity of a solid form is characterized by X-Ray Powder Diffraction (XRPD).

In some embodiments, the crystallinity of a solid form is determined by differential scanning calorimeter (DSC).

In some embodiments, the crystallinity of a solid form is determined by thermogravimetric analysis (TGA) in combination with XRPD and/or DSC.

In some embodiments, the tablet further comprises at least one coating layer. In some embodiments, the at least one coating layer comprises hydroxypropyl methylcellulose. In some embodiments, the at least one coating layer comprises at least one of titanium dioxide and polyethylene glycol.

In some embodiments, the at least one pharmaceutically acceptable excipient comprises at least one diluent. In some embodiments, the at least one diluent is chosen from mannitol, lactose monohydrate, anhydrous lactose, microcrystalline cellulose, starch, sorbitol, dextrose, dibasic calcium phosphate, dicalcium phosphate dihydrate, tricalcium phosphate, calcium phosphate, pregelatinized starch, compressible sugar, hydroxypropyl-methylcellulose, hydroxypropylmethylcellulose acetate stearate, sucrose-based diluents, confectioner's sugar, monobasic calcium sulfate monohydrate, calcium sulfate dihydrate, calcium lactate trihydrate, a dextrate, hydrolyzed cereal solids, amylose, powdered cellulose, calcium carbonate, glycine, kaolin, sodium chloride, inositol, and bentonite.

In some embodiments, the at least one diluent is present in a total amount of diluent ranging from about 45 to about 90% by weight of the tablet. In some embodiments, the at least one diluent is present in a total amount of diluent ranging from about 75 to about 90% by weight of the tablet.

In some embodiments, the at least one pharmaceutically acceptable excipient comprises at least one glidant. In some embodiments, the at least one glidant is chosen from colloidal anhydrous silica, and colloidal silicon dioxide. In some embodiments, the at least one glidant is present in a total amount of glidant ranging from about 0.1 to about 5% by weight of the tablet.

In some embodiments, the at least one pharmaceutically acceptable excipient comprises at least one disintegrant. In some embodiments, the at least one disintegrant is chosen from sodium starch glycolate, croscarmellose sodium, corn starch, potato starch, pregelatinized starch, methylcrystalline cellulose, methylcellulose, croscarmellose, cross-linked sodium carboxymethyl-cellulose, cross-linked carboxymethylcellulose, cross-linked croscarmellose, crosspovidone, cross-linked polyvinylpyrrolidone, alginic acid, sodium alginate, magnesium aluminum silicate, agar, guar, locust bean, Karaya, pectin, tragacanth, bentonite, citrus pulp, and sodium lauryl sulfate. In some embodiments, the at least one disintegrant is present in a total amount of disintegrant ranging from about 1 to about 15% by weight of the tablet.

In some embodiments, the at least one pharmaceutically acceptable excipient comprises at least one lubricant. In some embodiments, the at least one lubricant is chosen from magnesium stearate, stearic acid, calcium hydroxide, talc, sodium stearyl fumerate, mineral oil, hydrogenated soybean oil, aluminum, calcium, magnesium, zinc, sodium stearate, glycerol, talc, wax, boric acid, sodium benzoate, sodium acetate, sodium chloride, leucine, polyethylene glycol, methoxypolyethylene glycol, sodium oleate, sodium benzoate, glyceryl behenate, magnesium lauryl sulfate, sodium lauryl sulfate, colloidal silica, corn starch, silicone oil, and surfactant. In some embodiments, the at least one lubricant is present in a total amount of lubricant ranging from about 0.1 to about 5% by weight of the tablet.

In some embodiments, the tablet comprises an intragranular core and an extragranular portion. In some embodiments, the intragranular core comprises at least one diluent, at least one disintegrant, at least one glidant, and at least one lubricant.

In some embodiments, the extragranular portion comprises at least one disintegrant and at least one lubricant.

In some embodiments, Compound 1 is present in at least 50% crystalline form, such as at least 60%, at least 70%, at least 80%, at least 90%, at least 95%, at least 96%, at least 97%, at least 98%, at least 99%, 99.5%, or 100% crystalline.

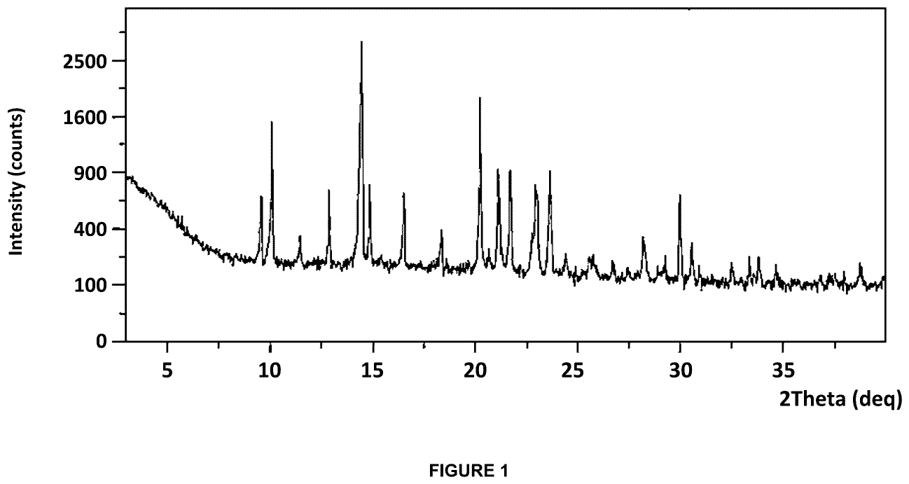

In some embodiments, Compound 1 is present as a solid form that is at least 50% crystalline Form A having an X-ray powder diffraction (XRPD) pattern derived using Cu (Kα) radiation comprising three, four, five, six, seven or more peaks, in term of 2-theta degrees, chosen from peaks at about 10.1±0.2, 14.3±0.2, 14.8±0.2, 16.4±0.2, 18.2±0.2, 20.1±0.2, 21.0±0.2, 21.6±0.2, 22.8±0.2, 23.5±0.2, 28.1±0.2, 29.8±0.2. In some embodiments, crystalline Form A has an XRPD pattern derived using Cu (Kα) radiation, in term of 2-theta degrees, having peaks at about 14.3±0.2, 20.1±0.2, 21.6±0.2, 22.8±0.2, and 23.5±0.2. In some embodiments, crystalline Form A has an X-ray powder diffraction pattern that is substantially in accordance with that shown in FIG. 1.

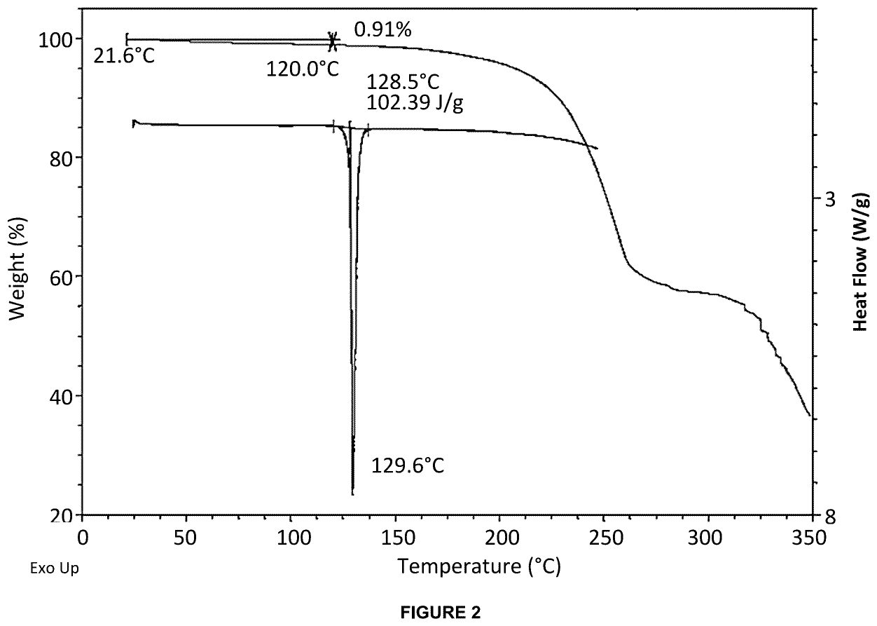

In some embodiments, crystalline Form A is characterized by a differential scanning calorimetry (DSC) curve with an onset at about 128.5° C. and an endothermic peak at 129.6° C. In some embodiments, crystalline Form A is characterized by a Thermogravimetric Analysis (TGA) profile with an about 0.91% w/w loss from about 21.6° C. to about 120° C. In some embodiments, crystalline Form A is characterized by a DCS/TGA profile substantially in accordance with that shown in FIG. 2.

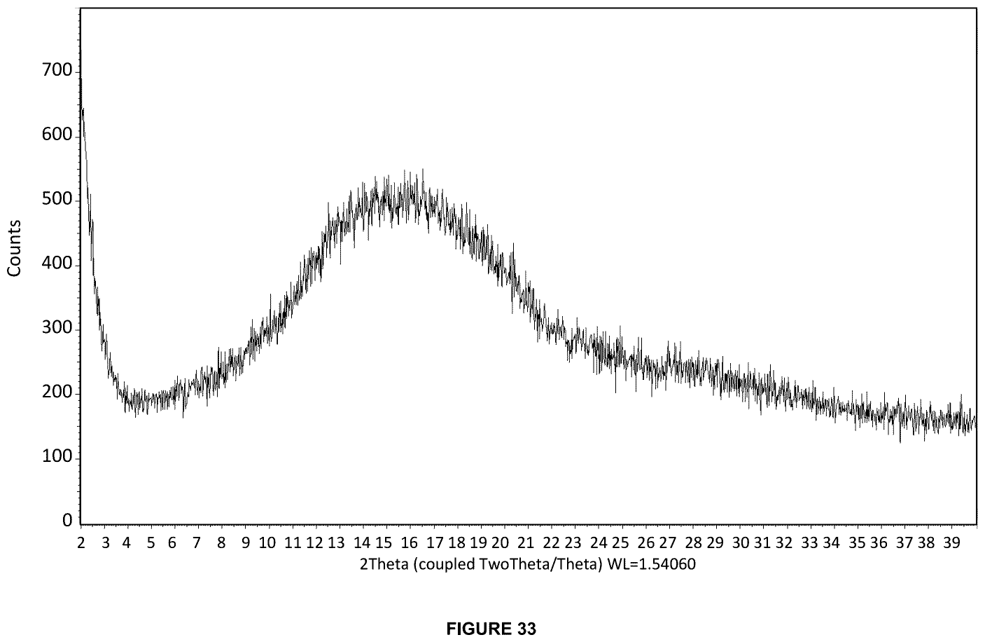

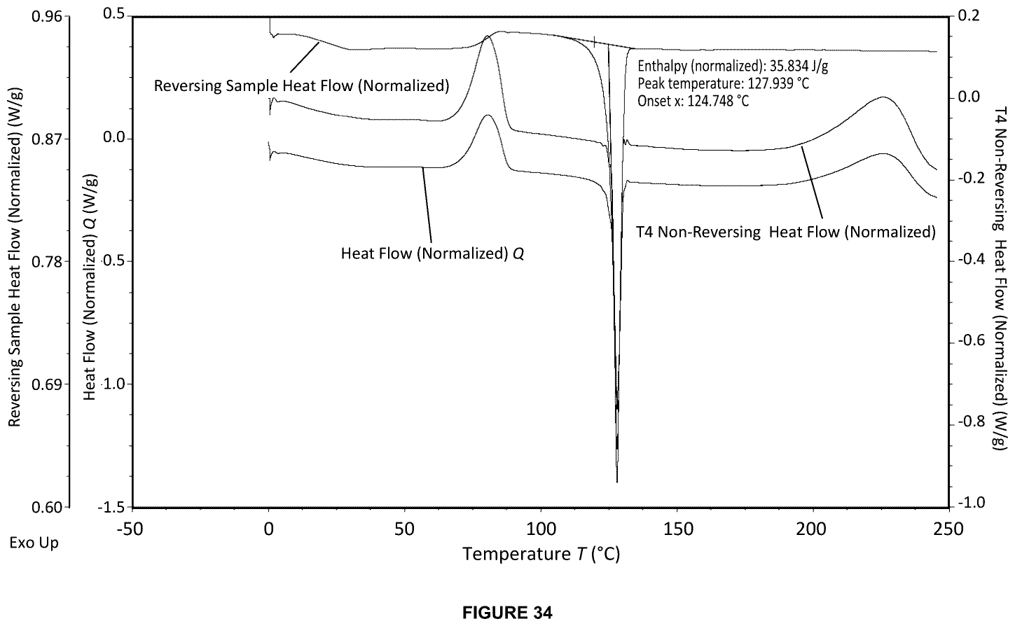

The present disclosure further relates to solid forms of 4-(3,3-difluoro-2,2-dimethyl-propanoyl)-3,5-dihydro-2H-pyrido[3,4-f][1,4]oxazepine-9-carbonitrile, characterized as amorphous. In some embodiments, the solid amorphous form of 4-(3,3-difluoro-2,2-dimethyl-propanoyl)-3,5-dihydro-2H-pyrido[3,4-f][1,4]oxazepine-9-carbonitrile is characterized by at least one of:
a) an X-ray powder diffraction (XRPD) pattern substantially in accordance with that shown in FIG. 33; or
b) a Differential Scanning calorimetry (DSC) thermogram having an onset at about 124.7° C. and a peak at about 127.9° C.

The present disclosure further relates to solid forms of 4-(3,3-difluoro-2,2-dimethyl-propanoyl)-3,5-dihydro-2H-pyrido[3,4-f][1,4]oxazepine-9-carbonitrile, characterized as polymorphous. In some embodiments, the tablet comprises a form of Compound 1 that is a polymorphic crystalline form. In some embodiments, the form of Compound 1 in the tablet is a polymorph of crystalline Form A. In some embodiments, the tablet comprises a substantially pure polymorphic crystalline form. In some embodiments, the tablet comprises a substantially pure polymorphic crystalline form of Form A. In some embodiments, the tablet comprises substantially pure crystalline Form A.

The present disclosure also relates to pharmaceutical compositions comprising any of the solid forms of Compound 1 disclosed herein and a pharmaceutically acceptable carrier.

In some embodiments, Compound 1 is present in an amount ranging from about 5 mg to about 60 mg. In some embodiments, Compound 1 is present in an amount of about 5 mg, about 10 mg, about 15 mg, about 20 mg, about 25 mg, about 30 mg, about 35 mg, about 40 mg, about 45 mg, about 50 mg, about 55 mg, or about 60 mg. In some embodiments, Compound 1 is present in an amount of about 20 mg.

In some embodiments, the tablet comprises 45 to 65% by weight anhydrous lactose, 20 to 40% by weight microcrystalline cellulose, 1 to 10% by weight sodium starch glycolate, 0.1 to 5% by weight colloidal silicon dioxide, and 0.1 to 5% by weight magnesium stearate.

In some embodiments, the tablet comprises 20 to 30% by weight mannitol, 50 to 70% by weight lactose monohydrate, 0.5 to 8% by weight sodium starch glycolate, 0.1 to 1% by weight colloidal anhydrous silica, 0.1 to 1% by weight colloidal silicon dioxide, and 0.1 to 5% by weight magnesium stearate.

In some embodiments, the tablet further comprises at least one of a pH adjusting agent, a salt, an antifoaming agent, an antioxidant, a preservative, a dispersing agent, a flavoring agent, a solubilizer, a plasticizer, a suspending agent, a surfactant, a viscosity enhancing agent, and a wetting agent.

In some embodiments, the tablet has a hardness of 60 N to 110 N. In some embodiments, the tablet has a hardness of 85 N.

In some embodiments, the tablet comprises less than about 1.0%, such as less than about 0.1%, about 0.09%, about 0.08%, about 0.07%, and about 0.06%, of a compound having the structure:

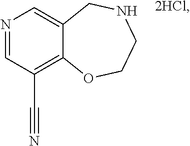

based on area percentage by HPLC analysis.

In some embodiments, the tablet comprises less than about 1.0%, such as less than about 0.1%, about 0.09%, about 0.08%, about 0.07%, and about 0.06%, of a compound having the structure:

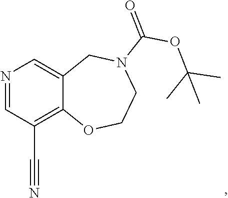 [SMILES: CC(C)(C)OC(=O)N1CCOc2c(C#N)cncc2C1]

based on area percentage by HPLC analysis.

In some embodiments, the tablet comprises less than about 1.0%, such as less than about 0.1%, about 0.09%, about 0.08%, about 0.07%, and about 0.06%, of a compound having the structure:

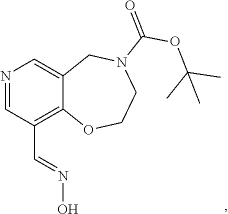 [SMILES: CC(C)(C)OC(=O)N1CCOc2c(/C=N/O)cncc2C1]

based on area percentage by HPLC analysis.

In some embodiments, the tablet comprises less than about 1.0%, such as less than about 0.1%, about 0.09%, about 0.08%, about 0.07%, and about 0.06%, of at least one of the compounds chosen from compounds having the structures:

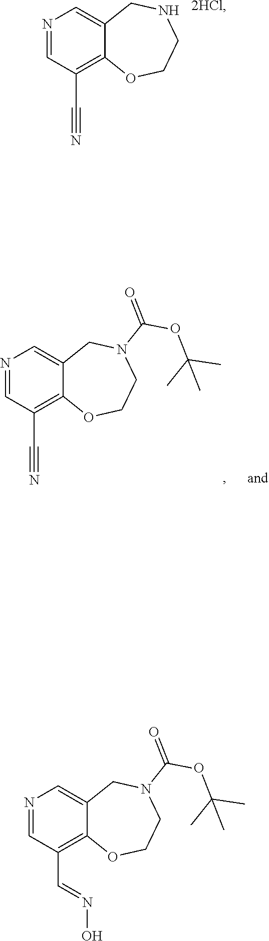

based on area percentage by HPLC analysis.

In some embodiments, the tablet comprises less than about 1.0%, such as less than about 0.1, about 0.09%, about 0.08%, about 0.07%, and about 0.06%, of total organic impurities comprising at least one of the compounds chosen from compounds having the structures:

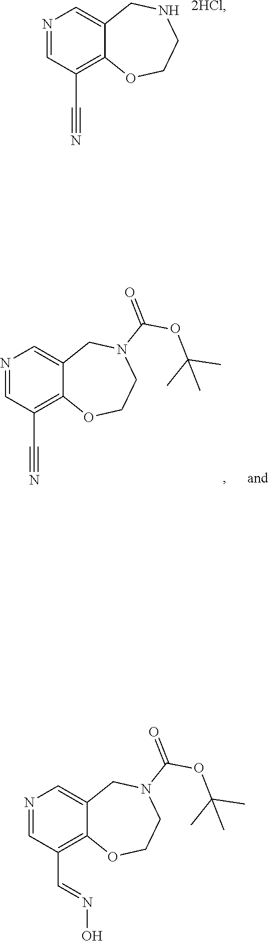

based on area percentage by HPLC analysis.

In some embodiments, the tablet comprises less than about 1.0% total organic impurities based on area percentage by HPLC analysis. In some embodiments, the tablet comprises less than about 0.2% total organic impurities based on area percentage by HPLC analysis. In some embodiments, the tablet comprises less than about 0.1% total organic impurities based on area percentage by HPLC analysis. In some embodiments, the tablet comprises less than about 0.09, 0.08, 0.07, 0.06, 0.05, 0.04, or 0.03% total organic impurities based on area percentage by HPLC analysis.

In some embodiments, the tablet comprises less than about 1.0% total impurities based on area percentage by HPLC analysis. In some embodiments, the tablet comprises less than about 0.2% total impurities based on area percentage by HPLC analysis. In some embodiments, the tablet comprises less than about 0.1% total impurities based on area percentage by HPLC analysis. In some embodiments, the tablet comprises less than about 0.09, 0.08, 0.07, 0.06, 0.05, 0.04, or 0.03% total impurities based on area percentage by HPLC analysis.

In some embodiments, the tablet comprises a polymorph, such as crystalline Form A, that comprises less than about 1.0%, such as less than about 0.1%, of a compound having the structure:

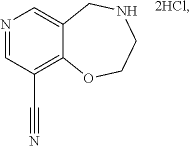

based on area percentage by HPLC analysis.

In some embodiments, the tablet comprises a polymorph, such as crystalline Form A, that comprises less than about 1.0%, such as less than about 0.1%, of a compound having the structure:

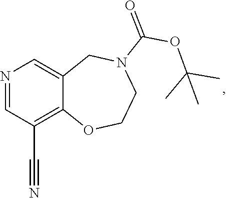 [SMILES: CC(C)(C)OC(=O)N1CCOc2c(C#N)cncc2C1]

based on area percentage by HPLC analysis.

In some embodiments, the tablet comprises a polymorph, such as crystalline Form A, that comprises less than about 1.0%, such as less than about 0.1%, of a compound having the structure:

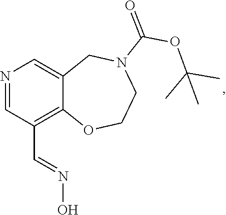 [SMILES: CC(C)(C)OC(=O)N1CCOc2c(/C=N/O)cncc2C1]

based on area percentage by HPLC analysis.

In some embodiments, the tablet comprises a polymorph, such as crystalline Form A, that comprises less than about 1.0%, such as less than about 0.1%, about 0.09%, about 0.08%, about 0.07%, and about 0.06%, of at least one of the compounds chosen from compounds having the structures:

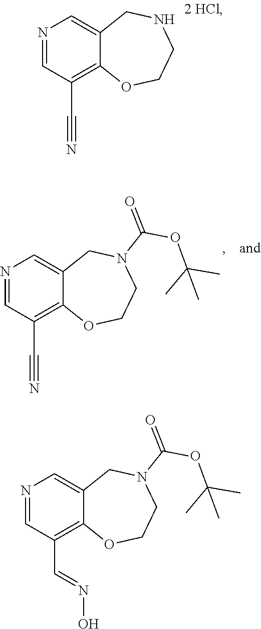 [SMILES: N#Cc1cncc2c1OCCNC2 ; CC(C)(C)OC(=O)N1CCOc2c(C#N)cncc2C1 ; CC(C)(C)OC(=O)N1CCOc2c(/C=N/O)cncc2C1]

based on area percentage by HPLC analysis.

In some embodiments, the tablet comprises a polymorph, such as crystalline Form A, that comprises less than about 1.0%, such as less than about 0.1, about 0.09%, about 0.08%, about 0.07%, and about 0.06%, of total organic impurities comprising at least one of the compounds chosen from compounds having the structures:

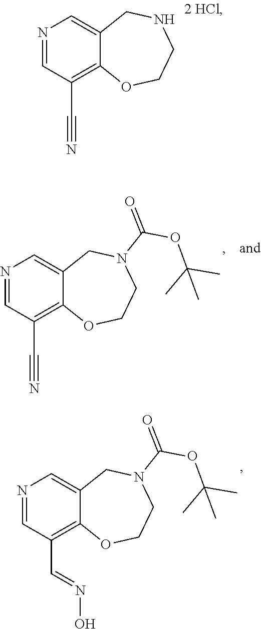 [SMILES: N#Cc1cncc2c1OCCNC2 ; CC(C)(C)OC(=O)N1CCOc2c(C#N)cncc2C1 ; CC(C)(C)OC(=O)N1CCOc2c(/C=N/O)cncc2C1]

based on area percentage by HPLC analysis.

The present disclosure further relates to solid dosage forms of 4-(3,3-difluoro-2,2-dimethyl-propanoyl)-3,5-dihydro-2H-pyrido[3,4-f][1,4]oxazepine-9-carbonitrile and any pharmaceutically acceptable salt thereof. In some embodiments, the solid dosage form comprises Compound 1 that is a polymorphic crystalline form. In some embodiments, the solid dosage form comprises a polymorph of crystalline Form A. In some embodiments, the solid dosage form comprises a substantially pure polymorph. In some embodiments, the solid dosage form comprises a substantially pure polymorphic crystalline form of Form A. In some embodiments, the solid dosage form comprises substantially pure crystalline Form A. In some embodiments, the solid dosage form comprises Compound 1 that is at least 50% crystalline, such as at least 60%, at least 70%, at least 80%, at least 90%, at least 95%, at least 96%, at least 97%, at least 98%, at least 99%, 99.5%, or 100% crystalline. In some embodiments, the solid dosage form comprises at least 90% crystalline Form A.

In some embodiments, the solid dosage form comprises Compound 1 that is at least 50% crystalline Form A having an X-ray powder diffraction (XRPD) pattern derived using Cu (Kα) radiation comprising three, four, five, six, seven or more peaks, in term of 2-theta degrees, chosen from peaks at about 10.1±0.2, 14.3±0.2, 14.8±0.2, 16.4±0.2, 18.2±0.2, 20.1±0.2, 21.0±0.2, 21.6±0.2, 22.8±0.2, 23.5±0.2, 28.1±0.2, 29.8±0.2. In some embodiments, the solid dosage form comprises crystalline Form A that has an XRPD pattern derived using Cu (Kα) radiation, in term of 2-theta degrees, having peaks at about 14.3±0.2, 20.1±0.2, 21.6±0.2, 22.8±0.2, and 23.5±0.2. In some embodiments, the solid dosage form comprises crystalline Form A having an X-ray powder diffraction pattern that is substantially in accordance with that shown in FIG. 1.

In some embodiments, the solid dosage form comprises less than about 1.0%, such as less than about 0.1%, of a compound having the structure:

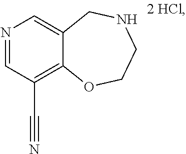 [SMILES: N#Cc1cncc2c1OCCNC2]

based on area percentage by HPLC analysis.

In some embodiments, the solid dosage form comprises less than about 1.0%, such as less than about 0.1%, of a compound having the structure:

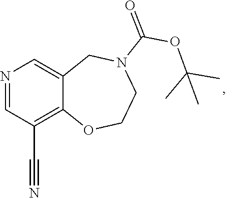 [SMILES: CC(C)(C)OC(=O)N1CCOc2c(C#N)cncc2C1]

based on area percentage by HPLC analysis.

In some embodiments, the solid dosage form comprises less than about 1.0%, such as less than about 0.1%, of a compound having the structure:

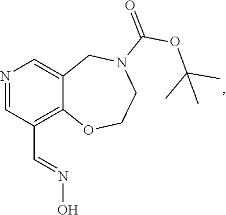 [SMILES: CC(C)(C)OC(=O)N1CCOc2c(/C=N/O)cncc2C1]

based on area percentage by HPLC analysis.

In some embodiments, the solid dosage form comprises less than about 1.0%, such as less than about 0.1%, about 0.09%, about 0.08%, about 0.07%, and about 0.06%, of at least one of the compounds chosen from compounds having the structures:

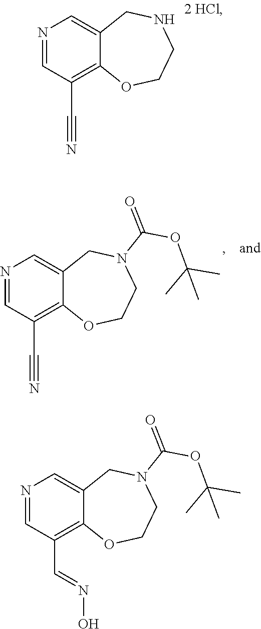 [SMILES: N#Cc1cncc2c1OCCNC2 ; CC(C)(C)OC(=O)N1CCOc2c(C#N)cncc2C1 ; CC(C)(C)OC(=O)N1CCOc2c(/C=N/O)cncc2C1]

based on area percentage by HPLC analysis.

In some embodiments, the solid dosage form comprises less than about 1.0%, such as less than about 0.1, about 0.09%, about 0.08%, about 0.07%, and about 0.06%, of total organic impurities comprising at least one of the compounds chosen from compounds having the structures:

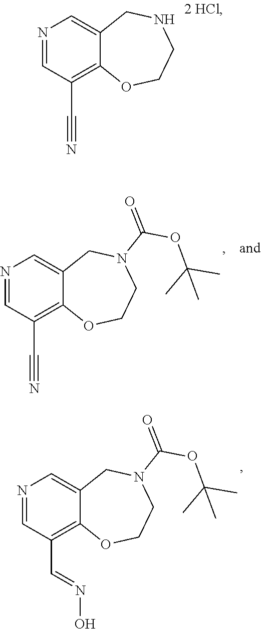 [SMILES: N#Cc1cncc2c1OCCNC2 ; CC(C)(C)OC(=O)N1CCOc2c(C#N)cncc2C1 ; CC(C)(C)OC(=O)N1CCOc2c(/C=N/O)cncc2C1]

based on area percentage by HPLC analysis.

The present disclosure also relates to a process for preparing a tablet comprising Compound 1. In some embodiments, a tablet is prepared by steps comprising: mixing Compound 1 with at least one excipient to obtain a homogenous blend, compressing the blend into a tablet, and spraying the tablet with a film-coating suspension. In some embodiments, the at least one excipient is selected from lactose monohydrate, mannitol, sodium starch glycolate, colloidal anhydrous silica, and magnesium stearate. In some embodiments, the process further comprises one or more sieving steps. In some embodiments, the process further comprises a step of dry roller compaction to produce granules. In some embodiments, the granules are mixed with one or more excipients prior to compression into a tablet.

The present disclosure also relates to a pharmaceutical formulation comprising Compound 1 and at least one pharmaceutically acceptable excipient, wherein the at least one pharmaceutically acceptable excipient comprises:
at least one diluent selected from the group consisting of lactose, sucrose, dextrose, dextrates, maltodextrin, mannitol, xylitol, sorbitol, cyclodextrins, calcium phosphate, calcium sulfate, starches, modified starches, cellulose, microcrystalline cellulose, microcellulose, and talc;
at least one disintegrating agent selected from the group consisting of natural starch, a pregelatinized starch, sodium starch, methylcrystalline cellulose, methylcellulose, croscarmellose, croscarmellose sodium, cross-linked sodium carboxymethylcellulose, cross-linked carboxymethylcellulose, cross-linked croscarmellose, cross-linked starch, cross-linked polymer, cross-linked polyvinylpyrrolidone, sodium alginate, clay, and gum; and
at least one binder selected from the group consisting of hydroxypropyl cellulose and polyvinylpyrrolidone; sodium lauryl sulfate; silica; and magnesium stearate;
   wherein the pharmaceutical formulation is chosen from tablet or capsule fill and Compound 1 is present in an amount ranging from about 5% to about 15% by weight of the tablet or capsule fill.

The present disclosure further relates to a pharmaceutical formulation comprising Compound 1 and at least one pharmaceutically acceptable excipient, wherein the formulation is in a form chosen from tablet, stock granulation, and capsule forms; wherein Compound 1 is present in an amount to provide a daily dose ranging from about 5 to about 60 mg per day in single or divided doses or multiple doses, wherein Compound 1 is present in an amount ranging from 5% to 50% by weight of tablet, stock granulation, or capsule fill; and further wherein the at least one pharmaceutically acceptable excipient comprises:
at least one diluent comprising at least one of anhydrous lactose, mannitol, and lactose monohydrate in a total amount of diluent ranging from 45% to 90% by weight of tablet or capsule fill;
at least one disintegrating agent comprising sodium starch glycolate in a total amount of disintegrant ranging from 2% to 10% by weight of tablet or capsule fill;
at least one glidant comprising at least one of silicon dioxide and colloidal anhydrous silica in a total amount of glidant ranging from 0.1% to 2% by weight of tablet or capsule fill; and
at least one lubricant comprising magnesium stearate in a total amount of lubricant ranging from 0.1% to 2% by weight of tablet or capsule fill.

In some embodiments, a blister pack contains the tablet or pharmaceutical formulation. In some embodiments, a bottle contains the tablet or pharmaceutical formulation.

In some embodiments, a liquid suspension comprises a crushed tablet or the dissolved contents of the pharmaceutical formulation. In some embodiments, the liquid suspension is prepared by crushing the tablet and adding the crushed tablet to water.

In some embodiments, a method of treating a disease or condition mediated by RIPK1 in a patient in need thereof, comprises administering to the patient the tablet, the pharmaceutical formulation, or the liquid suspension.

In some embodiments, the tablet, the pharmaceutical formulation, or the liquid suspension is used for treating a disease involving mediation of the RIPK1 receptor.

### II. RIPK1 Inhibitor Compound

The RIPK1 inhibitor compound can be prepared according to the methods and schemes described in, e.g., U.S. Pat. No. 11,203,600, in particular Method I, set forth at columns 63-66, which is incorporated herein by reference. Crystalline and/or amorphous forms of Compound 1 can also be prepared as described herein.

### III. Tablet Formulation

A tablet is provided that includes Compound 1 or a pharmaceutically acceptable salt thereof. In some embodiments, the tablet includes at least one pharmaceutically acceptable excipient.

In some embodiments the tablet comprises at least one diluent. Diluents are chemical compounds that are used to dilute the compound of interest prior to delivery. Diluents can also be used to stabilize compounds because they can provide a more stable environment Salts dissolved in buffered solutions (which also can provide pH control or maintenance) are utilized as diluents in the art, including, but not limited to a phosphate buffered saline solution. In some embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pack (Amstar); hydroxypropyl-methylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, spray-dried mannitol, sodium chloride; inositol, bentonite, and the like.

In some embodiments, the tablet includes at least one diluent is chosen from mannitol, spray-dried mannitol, lactose monohydrate, anhydrous lactose, microcrystalline cellulose, starch, sorbitol, dextrose, dibasic calcium phosphate, dicalcium phosphate dihydrate, tricalcium phosphate, calcium phosphate, pregelatinized starch, compressible sugar, hydroxypropyl-methylcellulose, hydroxypropylmethylcellulose acetate stearate, sucrose-based diluents, confectioner's sugar, monobasic calcium sulfate monohydrate, calcium sulfate dihydrate, calcium lactate trihydrate, a dextrate, hydrolyzed cereal solids, amylose, powdered cellulose, calcium carbonate, glycine, kaolin, sodium chloride, inositol, and bentonite.

In some embodiments, the tablet includes at least one diluent is chosen from mannitol, spray-dried mannitol, lactose monohydrate, anhydrous lactose, and microcrystalline cellulose. In some embodiments, the tablet includes at least one diluent is chosen from mannitol and lactose monohydrate. In some embodiments, the tablet includes at least one diluent chosen from anhydrous lactose and microcrystalline cellulose. In some embodiments, the tablet includes mannitol and lactose monohydrate. In some embodiments, the tablet includes anhydrous lactose and microcrystalline cellulose.

The total amount of diluent present in the tablet ranges from about 45 to about 90% by weight. In some embodiments, total amount of diluent present in the tablet ranges from about 75 to about 90% by weight. In some embodiments, the total amount of diluent present in the tablet ranges from about 80 to about 90% by weight. In some embodiments, the total amount of diluent present in the tablet is about 80% by weight, about 85% by weight, or about 90% by weight.

In some embodiments, the tablet includes microcrystalline cellulose. In some embodiments, the amount of microcrystalline cellulose in the tablet ranges from about 15 to about 45% by weight. In some embodiments, the amount of microcrystalline cellulose in the tablet ranges from about 20 to about 40% by weight, about 20 to about 35% by weight, about 25 to about 35% by weight, or about 25 to about 30% by weight. In some embodiments, the amount of microcrystalline cellulose in the tablet is about 29% by weight.

In some embodiments, the tablet includes anhydrous lactose. In some embodiments, the amount of anhydrous lactose in the tablet ranges from about 40 to about 70% by weight. In some embodiments, the amount of anhydrous lactose in the tablet ranges from about 45 to about 65% by weight, about 50 to about 65% by weight, about 45 to about 60% by weight, or about 50 to about 60% by weight. In some embodiments, the amount of anhydrous lactose in the tablet is about 55% by weight.

In some embodiments, the tablet includes mannitol. In some embodiments, the amount of mannitol in the tablet ranges from about 15 to about 45% by weight. In some embodiments, the amount of mannitol in the tablet ranges from about 20 to about 40% by weight, about 20 to about 35% by weight, about 25 to about 35% by weight, or about 20 to about 30% by weight. In some embodiments, the amount of mannitol in the tablet is about 25.5% by weight.

In some embodiments, the tablet includes spray-dried mannitol. In some embodiments, the amount of spray-dried mannitol in the tablet ranges from about 15 to about 45% by weight. In some embodiments, the amount of spray-dried mannitol in the tablet ranges from about 20 to about 40% by weight, about 20 to about 35% by weight, about 25 to about 35% by weight, or about 20 to about 30% by weight. In some embodiments, the amount of spray-dried mannitol in the tablet is about 25.5% by weight.

In some embodiments, the tablet includes lactose monohydrate. In some embodiments, the amount of lactose monohydrate in the tablet ranges from about 40 to about 70% by weight. In some embodiments, the amount of lactose monohydrate in the tablet ranges from about 45 to about 65% by weight, about 50 to about 65% by weight, about 45 to about 60% by weight, or about 55 to about 65% by weight. In some embodiments, the amount of lactose monohydrate in the tablet is about 59% by weight.

In some embodiments, the tablet includes spray-dried lactose monohydrate. In some embodiments, the amount of spray-dried lactose monohydrate in the tablet ranges from about 40 to about 70% by weight. In some embodiments, the amount of spray-dried lactose monohydrate in the tablet ranges from about 45 to about 65% by weight, about 50 to about 65% by weight, about 45 to about 60% by weight, or about 55 to about 65% by weight. In some embodiments, the amount of spray-dried lactose monohydrate in the tablet is about 59% by weight.

In some embodiments, the tablet comprises at least one glidant. In some embodiments, the at least one glidant is chosen from colloidal anhydrous silica, and colloidal silicon dioxide. In some embodiments, the tablet contains colloidal anhydrous silica, and colloidal silicon dioxide. In some embodiments, the tablet contains colloidal silicon dioxide.

The total amount of glidant present in the tablet ranges from about 0.1 to about 5% by weight. In some embodiments, the total amount of glidant present in the tablet ranges from about 0.1 to about 3% by weight, about 0.1 to about 2% by weight, about 0.1 to about 1.5% by weight, or about 0.1 to about 1% by weight. In some embodiments, the total amount of glidant present in the tablet is about 0.25% by weight.

In some embodiments, the glidant is colloidal silicon dioxide and colloidal anhydrous silica. The amount of colloidal silicon dioxide and colloidal anhydrous silica present in the tablet ranges from about 0.1 to about 5% by weight. In some embodiments, the amount of colloidal silicon dioxide and colloidal anhydrous silica present in the tablet ranges from about 0.1 to about 3% by weight, about 0.1 to about 2% by weight, about 0.1 to about 1.5% by weight, or about 0.1 to about 1% by weight. In some embodiments, the amount of colloidal silicon dioxide and colloidal anhydrous silica present in the tablet is about 0.25% by weight.

In some embodiments, the tablet comprises at least one disintegrant. Disintegrants contribute to both the dissolution and dispersion of the dosage form when contacted with gastrointestinal fluid. Disintegration agents or disintegrants facilitate the breakup or disintegration of a substance. Examples of disintegration agents include a starch, e.g., e.g., a natural starch such as corn starch or potato starch, a pregelatinized starch such as National 1551 or sodium starch glycolate such as Primogel® or Explotab®, a cellulose such as a wood product, methylcrystalline cellulose, e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the at least one disintegrant is chosen from sodium starch glycolate, croscarmellose sodium, corn starch, potato starch, pregelatinized starch, methylcrystalline cellulose, methylcellulose, croscarmellose, cross-linked sodium carboxymethyl-cellulose, cross-linked carboxymethylcellulose, cross-linked croscarmellose, crosspovidone, cross-linked polyvinylpyrrolidone, alginic acid, sodium alginate, magnesium aluminum silicate, agar, guar, locust bean, Karaya, pectin, tragacanth, bentonite, citrus pulp, and sodium lauryl sulfate. In some embodiments, the tablet includes sodium starch glycolate.

The total amount of disintegrant in the tablet ranges from about 1 to about 15% by weight. In some embodiments, the total amount of disintegrant in the tablet ranges from about 1 to about 10% by weight, about 1 to about 8% by weight, about 4 to about 15% by weight, or about 4 to about 12% by weight. In some embodiments, the total amount of disintegrant in the tablet about 5% by weight or about 8% by weight.

In some embodiments, the tablet includes sodium starch glycolate. The amount of sodium starch glycolate in the tablet ranges from about 1 to about 15% by weight. In some embodiments, the amount of sodium starch glycolate in the tablet ranges from about 1 to about 10% by weight, about 1 to about 8% by weight, about 4 to about 15% by weight, or about 4 to about 12% by weight. In some embodiments, the amount of sodium starch glycolate in the tablet is about 5% by weight or about 8% by weight.

In some embodiments, the tablet may also comprise at least one lubricant, which are compounds that prevent, reduce, or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

In some embodiments, the at least one lubricant is chosen from magnesium stearate, stearic acid, calcium hydroxide, talc, sodium stearyl fumarate, mineral oil, hydrogenated soybean oil, aluminum, calcium, magnesium, zinc, sodium stearate, glycerol, talc, wax, boric acid, sodium benzoate, sodium acetate, sodium chloride, leucine, polyethylene glycol, methoxypolyethylene glycol, sodium oleate, sodium benzoate, glyceryl behenate, magnesium lauryl sulfate, sodium lauryl sulfate, colloidal silica, corn starch, silicone oil, and surfactant. In some embodiments, the at least one lubricant is magnesium stearate.

In some embodiments, the total amount of lubricant in the tablet ranges from about 0.1 to about 5% by weight. In some embodiments, the total amount of lubricant in the tablet ranges from about 0.1 to about 3% by weight, about 0.25 to about 3% by weight, about 0.5 to about 1.5% by weight, or about 0.75 to about 1.25% by weight. In some embodiments, the total amount of lubricant in the tablet is about 1.25% by weight. In some embodiments, the total amount of lubricant in the tablet is about 0.75% by weight.

In some embodiments, the tablet includes magnesium stearate. In some embodiments, the amount of magnesium stearate in the tablet ranges from about 0.1 to about 5% by weight. In some embodiments, the amount of magnesium stearate in the tablet ranges from about 0.1 to about 3% by weight, about 0.25 to about 3% by weight, about 0.5 to about 1.5% by weight, or about 0.75 to about 1.25% by weight. In some embodiments, the amount of magnesium stearate in the tablet is about 1.25% by weight. In some embodiments, the amount of magnesium stearate in the tablet is about 0.75% by weight.

In some embodiments, the tablet comprises an intragranular core and an extragranular portion. The intragranular core can include at least one diluent, at least one disintegrant, at least one glidant, and at least one lubricant.

In some embodiments, the intragranular core includes Compound 1, mannitol, lactose monohydrate, sodium starch glycolate, colloidal anhydrous silica, colloidal silicon dioxide, and magnesium stearate. In some embodiments, the extragranular portion includes sodium starch glycolate and magnesium stearate.

In some embodiments, the intragranular core includes Compound 1, spray-dried mannitol, spray-dried lactose monohydrate, sodium starch glycolate, colloidal anhydrous silica, colloidal silicon dioxide, and magnesium stearate. In some embodiments, the extragranular portion includes sodium starch glycolate and magnesium stearate.

In some embodiments, the intragranular core includes Compound 1, anhydrous lactose, microcrystalline cellulose, sodium starch glycolate, colloidal silicon dioxide, and magnesium stearate. In some embodiments, the extragranular portion includes sodium starch glycolate and magnesium stearate.

In some embodiments, Compound 1 is present in at least 50% crystalline form, such as at least 60%, at least 70%, at least 80%, at least 90%, at least 95%, at least 96%, at least 97%, at least 98%, at least 99%, 99.5%, or 100% crystalline.

In some embodiments, the tablet includes Compound 1 in an amount ranging from about 5 mg to about 60 mg. In some embodiments, Compound 1 is present in an amount of about 5 mg, about 10 mg, about 15 mg, about 20 mg, about 25 mg, about 30 mg, about 35 mg, about 40 mg, about 45 mg, about 50 mg, about 55 mg, or about 60 mg. In some embodiments, Compound 1 is present in an amount of about 20 mg.

In some embodiments, the tablet comprises 45 to 65% by weight anhydrous lactose, 20 to 40% by weight microcrystalline cellulose, 1 to 10% by weight sodium starch glycolate, 0.1 to 5% by weight colloidal silicon dioxide, and 0.1 to 5% by weight magnesium stearate. In some embodiments, the tablet comprises 55% by weight anhydrous lactose, 29% by weight microcrystalline cellulose, 5% by weight sodium starch glycolate, 0.25% by weight colloidal silicon dioxide, and 0.75% by weight magnesium stearate.

In some embodiments, the tablet comprises 20 to 30% by weight mannitol, 50 to 70% by weight lactose monohydrate, 0.5 to 8% by weight sodium starch glycolate, 0.1 to 1% by weight colloidal anhydrous silica, 0.1 to 1% by weight colloidal silicon dioxide, and 0.1 to 5% by weight magnesium stearate. In some embodiments, the tablet comprises 25.5% by weight mannitol, 59% by weight lactose monohydrate, 4% by weight sodium starch glycolate, 0.25% by weight colloidal anhydrous silica and colloidal silicon dioxide, and 1.25% by weight magnesium stearate.

In some embodiments, the tablet comprises 20 to 30% by weight spray-dried mannitol, 50 to 70% by weight spray-dried lactose monohydrate, 0.5 to 8% by weight sodium starch glycolate, 0.1 to 1% by weight colloidal anhydrous silica, 0.1 to 1% by weight colloidal silicon dioxide, and 0.1 to 5% by weight magnesium stearate. In some embodiments, the tablet comprises 25.5% by weight mannitol, 59% by weight lactose monohydrate, 4% by weight sodium starch glycolate, 0.25% by weight colloidal anhydrous silica and colloidal silicon dioxide, and 1.25% by weight magnesium stearate.

In some embodiments, the tablet may include at least one pH adjusting agent and/or buffering agent, for example, acids such as acetic, citric, fumaric, maleic, tartaric, malic, lactic, phosphoric and hydrochloric acids; bases such as sodium hydroxide, sodium phosphate, sodium borate, sodium citrate, sodium lactate and tris-hydroxymethylaminomethane; and buffers such as citrate, sodium bicarbonate, ammonium chloride, and the like. Such buffers used as bases may have other counterions than sodium, for example, potassium, magnesium, calcium, ammonium, or other counterions. Such acids, bases and buffers are included in an amount required to maintain pH of the composition in an acceptable range.

In some embodiments, the tablet can have a hardness of 60 N to 110 N. In some embodiments, the tablet can have a hardness of 70 N to 100 N. In some embodiments, the tablet can have a hardness of 75 N to 95 N. In some embodiments, the tablet can have a hardness of 80 N to 90 N. In some embodiments, the tablet has a hardness of 60 N, 65 N, 70 N, 75 N, 80 N, 85 N, 90 N, 95 N, 100 N, 105 N, or 110 N. In some embodiments, the tablet has a hardness of 85 N. The hardness of a tablet can be measured using a method described according to European pharmacopoeia 10.0 page 336-337.

In some embodiments, the tablet may also include at least one antifoaming agent to reduce foaming during processing which can result in coagulation of aqueous dispersions, bubbles in the finished film, or generally impair processing. Exemplary anti-foaming agents include silicon emulsions or sorbitan sesquioleate.

In some embodiments, the tablet may also include at least one antioxidant, such as non-thiol antioxidants, for example, butylated hydroxytoluene (BHT), sodium ascorbate, ascorbic acid or its derivative, and tocopherol or its derivatives. In certain embodiments, antioxidants enhance chemical stability where required. Other agents such as citric acid or citrate salts or EDTA may also be added to slow oxidation.

In some embodiments, the tablet may also include at least one preservative to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

In some embodiments, the tablet may also include at least one dispersing agent and/or viscosity modulating agent. Dispersing agents and/or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween®60 or 80, polyvinylpyrrolidone (PVP; commercially known as Plasdone®), triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F10®8, which are block copolymers of ethylene oxide and propylene oxide); and poloxamines (e.g., Tetronic 908®, also known as Poloxamine 908®, which is a tetrafonctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polysorbate-80, gums, such as, e.g., gum tragacanth and gum acacia, guar gum, xanthans, including xanthan gum, polyethoxylated sorbitan monolaurate, polyethoxylated sorbitan monolaurate, and carbomers. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. In general, binder levels of about 10 to about 70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 90% and more typically up to 70% in tablet formulations is common.

In some embodiments, the tablet may also include at least one erosion facilitator. Erosion facilitators include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

In some embodiments, the tablet may also include at least one filling agent, which includes compounds such as dextrates, dextran, sucrose, xylitol, lactitol, and the like.

In some embodiments, the tablet may also include at least one flavoring agent and/or sweetener e.g., acacia syrup, acesulfame K, alitame, anise, apple, aspartame, banana, Bavarian cream berry, black currant, butterscotch, calcium citrate, camphor, caramel, cherry, cherry cream chocolate, cinnamon, bubble gum, citrus, citrus punch, citrus cream, cotton candy, cocoa, cola, cool cherry, cool citrus, cyclamate, cyclamate, eucalyptus, eugenol, fructose, fruit punch, ginger, glycyrrhizinate, glycyrrhiza (licorice) syrup, grape, grapefruit, honey, isomalt, lemon, lime, lemon cream, monoammonium glycyrrhizinate, maltol, maple, marshmallow, menthol, mint cream, mixed berry, neohesperidine DC, neotame, orange, pear, peach, peppermint, peppermint cream, Powder, raspberry, root beer, rum, saccharin, safrole, spearmint, spearmint cream, strawberry, strawberry cream, stevia, sucralose, sucrose, sodium saccharin, saccharin, aspartame, acesulfame potassium, talin, xylitol, sucralose, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

In some embodiments, the tablet may also include at least one solubilizer, which includes compounds such as triacetin, triethylcitrate, ethyl oleate, ethyl caprylate, sodium doccusate, vitamin E TPGS, dimethylacetamide, N-methylpyrrolidone, N-hydroxyethylpyrrolidone, hydroxypropyl cyclodextrins for example Captisol®, ethanol, n-butanol, isopropyl alcohol, cholesterol, bile salts, glycofurol, transcutol, and dimethyl isosorbide and the like. In one embodiment, the solubilizer is vitamin E TPGS and/or Captisol® or ß-hydroxypropylcyclodextrin.

In some embodiments, the tablet may also include at least one suspending agent, which includes compounds such as vinyl pyrrolidone/vinyl acetate copolymer (S630), polysorbate-80, hydroxyethylcellulose, gums, such as, e.g., gum tragacanth and gum acacia, guar gum, xanthans, including xanthan gum, polyethoxylated sorbitan monolaurate, polyethoxylated sorbitan monoleate, and the like.

In some embodiments, the tablet may also include at least one surfactant, which includes compounds such as sodium docusate, Tween 20, 60 or 80, triacetin, vitamin E TPGS, sorbitan monooleate, polyoxyethylene sorbitan monooleate, polyoxyethylene sorbitan monolaurate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g. octoxynol 10, octoxynol 40. In some embodiments, surfactants may be included to enhance physical stability or for other purposes.

In some embodiments, the tablet may also include at least one wetting agent, which includes compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium doccusate, triacetin, Tween 80, vitamin E TPGS, ammonium salts and the like.

Pharmaceutical preparations disclosed herein can be obtained by mixing at least one solid excipient described herein, with Compound 1 described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as Hypromellose (i.e. hydroxypropyl methylcellulose). The capsules can contain the active ingredients, optionally in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, lipids, solubilizers, or liquid polyethylene glycols. In addition, stabilizers may be added. All formulations for oral administration should be in dosages suitable for such administration.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3^{rd} ed. (1986). Other methods include, e.g., spray drying, pan coating, melt granulation, granulation, fluidized bed spray drying or coating (e.g., wurster coating), tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipient that can be included in solid dosage forms described herein. The type and amounts of such excipient can be readily determined by one skilled in the art, according to the particular properties desired.

In some embodiments, the tablet includes at least one coating layer. The coating layer can include hydroxypropyl methylcellulose. In some embodiments, the coating layer comprises at least one of titanium dioxide and polyethylene glycol. In some embodiments, the coating layer comprises hydroxypropyl methylcellulose, titanium dioxide, and polyethylene glycol. In some embodiments, the coating layer comprises hydroxypropyl methylcellulose in an amount ranging from 55 to 70% by weight of the coating layer, titanium dioxide in an amount ranging from 20 to 30% by weight of the coating layer, and polyethylene glycol in an amount ranging from 6 to 15% by weight of the coating layer.

In some embodiments, the coating layer comprises about 1 to about 8% by weight of the tablet. In some embodiments, the coating layer comprises about 1% by weight, about 2% by weight, about 3% by weight, about 4% by weight, about 5% by weight, about 6% by weight, about 7% by weight, or about 8% by weight of the tablet. In some embodiments, the coating layer includes hydroxypropyl methylcellulose, titanium dioxide, and polyethylene glycol in an amount of about 2 to 4% by weight of the tablet. In some embodiments, the coating layer includes hydroxypropyl methylcellulose, titanium dioxide, and polyethylene glycol in an amount of about 3% by weight of the tablet.

In some embodiments, the pharmaceutical formulation is free of titanium dioxide.

Colorants, surfactants, anti-adhesion agents, antifoaming agents, lubricants (e.g., carnauba wax or PEG) and other additives may be added to the coatings besides plasticizers to solubilize or disperse the coating material, and to improve coating performance and the coated product.

In some embodiments, the tablets are packaged in a blister pack. The blister packs can be made from any appropriate material. Examples include polyvinyl-polychlorotrifluoroethylene in the forming film and hardened aluminum foil as lidding.

In some embodiments, the tablets are packaged in a bottle. The bottle can be made from any appropriate material.

### IV. Liquid Formulation

A liquid suspension comprising a crushed tablet is provided herein. A liquid suspension can be prepared by adding a tablet to water and crushing the tablet to disperse it in the water. In some embodiments, the liquid suspension is prepared by crushing the tablet before adding water to form the liquid suspension.

In some embodiments, a syringe is provided that includes a liquid suspension of a crushed tablet. The syringe can be used to connect to a gastric tube and empty it into a gastric tube.

### V. Therapeutic Methods

Provided herein are methods of treating a disease or condition mediated by RIPK1 comprising administering to a subject in need thereof a therapeutically effective amount of the RIPK1 inhibitor compound comprising 4-(3,3-difluoro-2,2-dimethyl-propanoyl)-3,5-dihydro-2H-pyrido[3,4-f][1,4]oxazepine-9-carbonitrile. In some embodiments the therapeutically effective amount is about 5 to about 60 mg. In some embodiments, the subject is a mammal. In some embodiments, the mammal is a human. In some embodiments, the subject has one or more symptoms of amyotrophic lateral sclerosis (ALS) prior to treatment and the treatment reduces or eliminates the one or more symptoms. In some embodiments, the subject suffers from neuropathic pain, musculoskeletal pain, or spasticity caused by ALS.

In some embodiments, a subject with ALS has at least one documented relapse within the previous year, and/or greater than two documented relapses within the previous two years, and/or greater than one active Gd-enhancing brain lesion on an MRI scan in the past six months and prior to screening.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg is administered. In some embodiments, the dose is about 5 mg, about 10 mg, about 15 mg, about 20 mg, about 25 mg, about 30 mg, about 35 mg, about 40 mg, about 45 mg, about 50 mg, about 55 mg, or about 60 mg. In some embodiments, the dose is about 5 mg. In some embodiments, the dose is about 10 mg. In some embodiments, the dose is about 15 mg. In some embodiments, the dose is about 20 mg. In some embodiments, the dose is about 30 mg. In some embodiments, the dose is about 40 mg. In some embodiments, the dose is about 60 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in a form of tablets. In some embodiments, the dose is administered in the form of pills, capsules, semisolids, powders, sustained release formulations, solutions, suspensions, elixirs, aerosols, or any other appropriate compositions.

In some embodiments, the subject is administered the RIPK1 inhibitor compound for a period of about 4, 8, 12, 16, 20 or 24 weeks. In some embodiments, the subject is administered the RIPK1 inhibitor compound for a period of about 12 weeks. In some embodiments, the dose is once daily.

In some embodiments, the dose is administered with food. In some embodiments, the dose is administered once daily with food. In some embodiments, the dose of 5 mg, 15 mg, 30 mg, 40 mg or 60 mg is administered with food. In some embodiments, the dose of 5 mg, 10 mg, 15 mg, 20 mg, 30 mg, 40 mg, or 60 mg is administered once daily with food. In some embodiments, the dose of 40 mg is administered once daily with food. In some embodiments, the dose is administered in oral solution or tablets. In some embodiments, the dose is administered in oral solution or tablets with food. In some embodiments, the dose is administered once daily in oral solution or tablets. In some embodiments, the dose is administered once daily in oral solution or tablets with food. In some embodiments, the dose of 40 mg is administered in oral solution or tablets. In some embodiments, the dose of 40 mg is administered in oral solution or tablets with food. In some embodiments, the dose of 40 mg is administered once daily in oral solution or tablets. In some embodiments, the dose of 40 mg is administered once daily in oral solution or tablets with food.

In some embodiments, the dose is administered with food. In some embodiments, the dose is administered twice daily with food. In some embodiments, the dose of 5 mg, 10 mg, 15 mg, 20 mg, or 30 mg is administered with food. In some embodiments, the dose of 5 mg, 10 mg, 15 mg, 20 mg, or 30 mg is administered twice daily with food. In some embodiments, the dose of 20 mg is administered twice daily with food. In some embodiments, the dose is administered in oral solution or tablets. In some embodiments, the dose is administered in oral solution or tablets with food. In some embodiments, the dose is administered twice daily in oral solution or tablets. In some embodiments, the dose is administered twice daily in oral solution or tablets with food.

In some embodiments, the dose is 10 mg administered once daily. In some embodiments, the dose is 20 mg administered once daily.

In some embodiments, the dose is 15 mg administered twice daily. In some embodiments, the dose is 20 mg administered twice daily.

In some embodiments, a method of treating amyotrophic lateral sclerosis (ALS) is provided, comprising administering to a subject in need thereof a RIPK1 inhibitor comprising 4-(3,3-difluoro-2,2-dimethyl-propanoyl)-3,5-dihydro-2H-pyrido[3,4-f] [1,4]oxazepine-9-carbonitrile. In some embodiments, a method of treating amyotrophic lateral sclerosis (ALS) is provided, comprising administering to a subject in need thereof a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily. In some embodiments, a method of treating amyotrophic lateral sclerosis (ALS) is provided, comprising administering to a subject in need thereof a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof a dose of about 5 to about 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile. In some embodiments, a method of treating amyotrophic lateral sclerosis (ALS) is provided, comprising administering to a subject in need thereof a dose of about 5 to about 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof a dose of about 5 to about 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily. In some embodiments, a method of treating amyotrophic lateral sclerosis (ALS) is provided, comprising administering to a subject in need thereof a dose of about 5 to about 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof a dose of about 5 to about 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 24 weeks.

In some embodiments, a method of treating amyotrophic lateral sclerosis (ALS) is provided, comprising administering to a subject in need thereof 5 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 10 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 15 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 20 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 30 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 5 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 10 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 15 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 20 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 30 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks.

In some embodiments, a method of treating amyotrophic lateral sclerosis (ALS) is provided, comprising administering to a subject in need thereof 5 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 10 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 15 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 20 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 30 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered for a period of at least about 12 weeks. In some embodiments, a method of treating amyotrophic lateral sclerosis (ALS) is provided, comprising administering to a subject in need thereof 5 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 10 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 15 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 20 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 30 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered once daily for a period of at least about 12 weeks. In some embodiments, a method of treating amyotrophic lateral sclerosis (ALS) is provided, comprising administering to a subject in need thereof 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered once daily for a period of at least about 12 weeks.

In some embodiments, a method of treating amyotrophic lateral sclerosis (ALS) is provided, comprising administering to a subject in need thereof 5 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered twice daily for a period of at least about 12 weeks. In some embodiments, a method of treating amyotrophic lateral sclerosis (ALS) is provided, comprising administering to a subject in need thereof 10 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered twice daily for a period of at least about 12 weeks. In some embodiments, a method of treating amyotrophic lateral sclerosis (ALS) is provided, comprising administering to a subject in need thereof 15 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered twice daily for a period of at least about 12 weeks. In some embodiments, a method of treating amyotrophic lateral sclerosis (ALS) is provided, comprising administering to a subject in need thereof 20 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile, wherein the RIPK1 inhibitor compound is administered twice daily for a period of at least about 12 weeks. In some embodiments, a method of treating amyotrophic lateral sclerosis (ALS) is provided, comprising administering to a subject in need thereof 30 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile, wherein the RIPK1 inhibitor compound is administered twice daily for a period of at least about 12 weeks.

In some embodiments, the RIPK1 inhibitor compound is administered as monotherapy. In some embodiments, the method comprises administering the RIPK1 inhibitor compound and at least one additional therapeutic agent. The additional therapeutic agent may be administered concurrently or sequentially with the RIPK1 inhibitor compound.

Determination of the frequency of administration can be made by persons skilled in the art, such as an attending physician based on considerations of the condition being treated, age of the subject being treated, severity of the condition being treated, general state of health of the subject being treated and the like. In some embodiments, RIPK1 inhibitor compounds are administered in a therapeutically effective amount for treatment of ALS. The therapeutically effective amount is typically dependent on the weight of the subject being treated, his or her physical or health condition, the extensiveness of the condition to be treated, or the age of the subject being treated, pharmaceutical formulation methods, and/or administration methods (e.g., administration time and administration route).

In some embodiments, a method of treating ALS is provided, the method comprising administering to a subject in need thereof a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile. In some embodiments, a method of treating ALS is provided, the method comprising administering to a subject in need thereof a dose of about 5 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile. In some embodiments, a method of treating ALS is provided, the method comprising administering to a subject in need thereof a dose of about 10 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile. In some embodiments, a method of treating ALS is provided, the method comprising administering to a subject in need thereof a dose of about 15 mg of a RIPK1 inhibitor comprising 4-(3,3-difluoro-2,2-dimethyl-propanoyl)-3,5-dihydro-2H-pyrido[3,4-f] [1,4]oxazepine-9-carbonitrile. In some embodiments, a method of treating ALS is provided, the method comprising administering to a subject in need thereof a dose of about 20 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile. In some embodiments, a method of treating ALS is provided, the method comprising administering in a subject in need thereof a dose of about 30 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile. In some embodiments, a method of treating ALS is provided, the method comprising administering to a subject in need thereof a dose of about 60 mg of a RIPK1 inhibitor comprising 4-(3,3-difluoro-2,2-dimethyl-propanoyl)-3,5-dihydro-2H-pyrido[3,4-f][1,4]oxazepine-9-carbonitrile.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

### EXAMPLES

The following examples are provided to illustrate certain disclosed embodiments and are not to be construed as limiting the scope of this disclosure in any way. In the Examples discussed below, the RIPK1 inhibitor, as defined above, may be also referred as “Compound 1”, “the compound” or “the drug” interchangeably.

As noted herein, Compound 1 can be prepared according to the methods and schemes described in, e.g., U.S. Pat. No. 11,203,600. Solid forms, including crystalline and amorphous forms, can also be prepared as described herein.

### Example 1: Crystalline Compound (1)

Compound (1) described herein may be made as described below.

The synthetic route is set forth below:

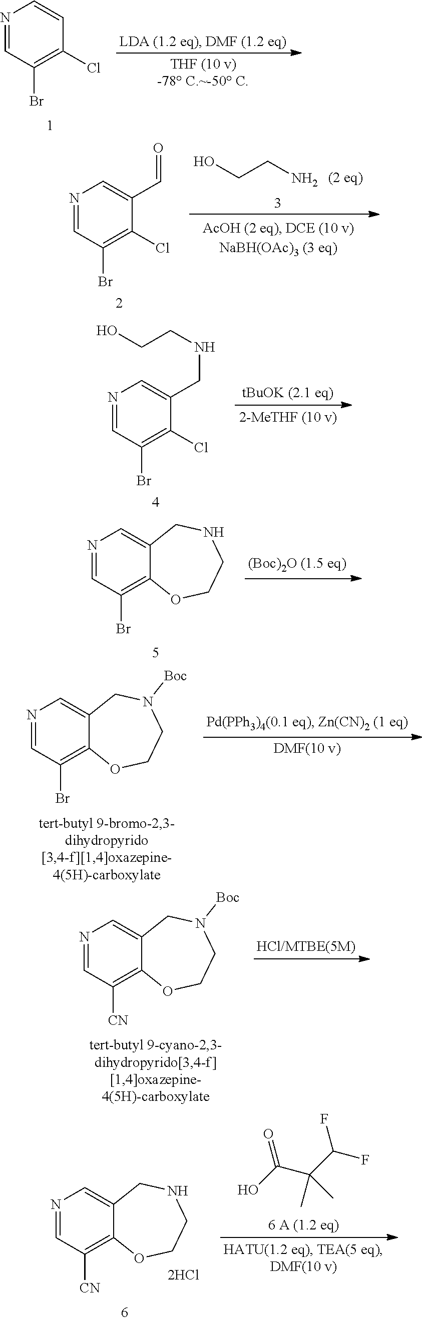

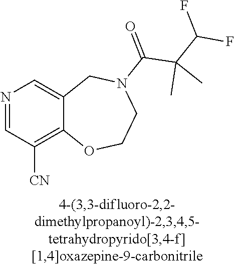 [SMILES: CC(C)(C(=O)N1CCOc2c(C#N)cncc2C1)C(F)F]

Compound 2 was made as follows:

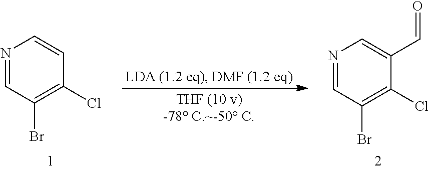 [SMILES: Clc1ccncc1Br ; O=Cc1cncc(Br)c1Cl]

Two reactions were carried out in parallel. To a solution of diisopropylamine (1.23 kg, 12.2 mol, 1.72 L, 1.2 eq) in THF (10 L) was added n-BuLi (2.5 M, 4.86 L, 1.2 eq) at −30° C. under N₂, and the mixture was stirred at −30° C. for 30 min. Then the mixture was added to a solution of compound 1 (1950 g, 10.13 mol, 1 eq) in THF (16 L) at −78° C. under N₂, and the reaction was stirred at −78° C. for 2.5 h. DMF (889 g, 12.2 mol, 936 mL, 1.2 eq) was added to the reaction mixture at −78° C., and the resulting mixture was stirred at −50° C. for 1 h. TLC (PE:EtOAc=5:1) indicated compound 1 was consumed completely and one new spot (Rf_{R1}=0.55, Rf_{P1}=0.50) formed. The reaction was clean according to TLC. The reaction mixture was quenched by addition of sat. aq. NH₄Cl (10 L), and the aqueous was extracted with EtOAc (5 L). The combined organic layers were washed with brine (10 L×1), dried over Na₂SO₄, filtered and concentrated under reduced pressure to give a residue. The crude product was dissolved with EtOAc (16 L), and filtered. The organic layers were washed with 1M HCl solution (2 L), and brine (2 L). The two batches were combined, dried over anhydrous Na₂SO₄, filtered and concentrated in vacuo to give compound 2 (2800 g, 12.7 mol, 62.7% yield) as a yellow solid without further purification.

¹H NMR: 400 MHZ CDCl₃ δ 10.46 (s, 1H), 8.92 (d, J=1.6 Hz, 2H).

Compound 4 was made as follows:

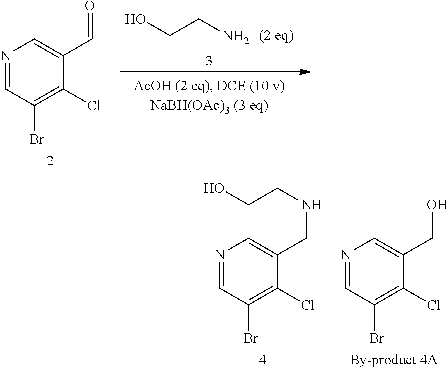

Two reactions were carried out in parallel. To a solution of compound 2 (1400 g, 6.35 mol, 1 eq) in DCE (14 L) was added compound 3 (776 g, 12.7 mol, 768 mL, 2 eq), followed by AcOH (1.14 kg, 19.1 mol, 1.09 L, 3 eq) at 0˜15° C. The mixture was stirred at 25° C. for 1 hr under N₂ atmosphere. NaBH(OAc)₃ (2.69 kg, 12.7 mol, 2 eq) was then added at 0˜15° C. and the reaction mixture was stirred at 25° C. for 12 h. TLC (DCM:MeOH=20:1) indicated compound 2 was consumed completely and one new spot (Rf_{P1}=0.33) formed. LC-MS showed no compound 2 remained. Several new peaks were shown on LC-MS (Retention time=1.2 min) and one main peak with desired mass was detected. The two batches were combined together for workup. The reaction mixture was diluted with water (10 L) and stirred 30 min. The layers were separated and the aqueous layer was extracted with DCM (2 L). The aqueous was added aqueous NaOH (5M) till pH to 9˜10. The aqueous was extracted with DCM (3×8 L). The combined organic layers were washed with brine (1×2 L), dried over Na₂SO₄, filtered and concentrated under reduced pressure to give compound 4 (2000 g, 7.28 mol, 57.34% yield, 96.7% purity) as a yellow solid, which was used in the next step without further purification.

¹H NMR: 400 MHZ CDCl₃ δ 8.66 (s, 1H), 8.49 (s, 1H), 3.97 (s, 2H), 3.61-3.78 (m, 3H), 2.72-2.86 (m, 2H).

Compound 5 was made as follows:

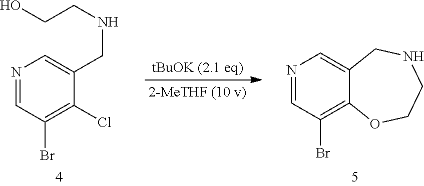 [SMILES: OCCNCc1cncc(Br)c1Cl ; Brc1cncc2c1OCCNC2]

To a solution of compound 4 (1050 g, 3.95 mol, 1 eq) in 2-methyltetrahydrofuran (10 L) was added t-BuOK (932 g, 8.30 mol, 2.1 eq) at 0˜20° C. The reaction mixture was stirred at 25° C. for 2 hr. LC-MS and HPLC showed that no compound 4 remained. Several new peaks were shown on LC-MS (Retention time=1.36 min) and one main peak with desired mass was detected. The reaction mixture was used in the next step directly.

### Preparation of tert-butyl 9-bromo-2,3-dihydropyrido[3,4-f][1,4]oxazepine-4(5H)-carboxylate

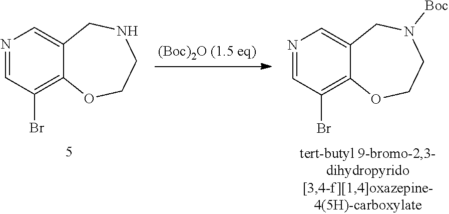 [SMILES: Brc1cncc2c1OCCNC2 ; CC(C)(C)OC(=O)N1CCOc2c(Br)cncc2C1]

Two reactions were carried out in parallel. Boc₂O (1.17 kg, 5.37 mol, 1.23 L, 1.5 eq) was added to the reaction mixture of compound 5 (819.55 g, 3.58 mol, 1 eq) at 25° C., and the mixture was stirred at 25° C. for 16 h under N₂ atmosphere. LC-MS showed no compound 5 remained. Several new peaks were shown on LC-MS (Retention time=1.31 min) and one main peak with desired mass was detected. The two batches were combined together. The reaction mixture was added water (15 L) at 15° C., and the aqueous was extracted with EtOAc (3 L×2). The combined organic layers were washed with brine (5 L), dried over Na₂SO₄, filtered and concentrated. The residue was purified by column chromatography (SiO₂, Petroleum ether/Ethyl acetate=15:1 to 1:1, PE:EtOAc=3:1, Rf_{P1}=0.43) to give tert-butyl 9-bromo-2,3-dihydropyrido[3,4-f][1,4]oxazepine-4(5H)-carboxylate (1520 g, 100% purity) as an off-white solid.

¹H NMR: 400 MHz CDCl₃ δ 8.59 (br s, 1H), 8.24-8.38 (m, 1H), 4.45-4.64 (m, 2H), 4.26 (br s, 2H), 3.84-3.91 (m, 2H), 1.43 (s, 9H).

Procedure for preparation of tert-butyl 9-cyano-2,3-dihydropyrido[3,4-f][1,4]oxazepine-4(5H)-carboxylate

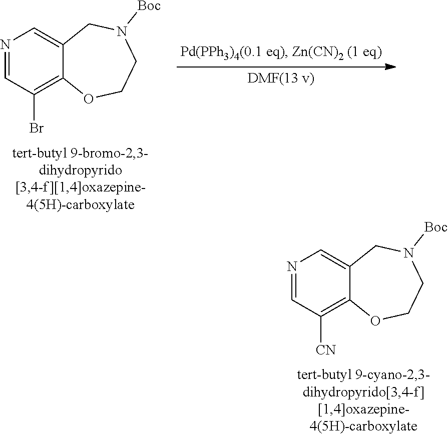 [SMILES: CC(C)(C)OC(=O)N1CCOc2c(Br)cncc2C1 ; CC(C)(C)OC(=O)N1CCOc2c(C#N)cncc2C1]

Five reactions were carried out in parallel. A mixture of tert-butyl 9-bromo-2,3-dihydropyrido[3,4-f][1,4]oxazepine-4(5H)-carboxylate (200 g, 608 mmol, 1 eq), Pd(PPh₃)₄ (70.2 g, 60.8 mmol, 0.1 eq), Zn(CN)₂ (74.9 g, 638 mmol, 40.5 mL, 1.05 eq) in DMF (2 L) was degassed and purged with N₂ for 3 times, and the mixture was stirred at 110° C. for 16 hr under N₂ atmosphere. LC-MS showed no tert-butyl 9-bromo-2,3-dihydropyrido[3,4-f][1,4]oxazepine-4(5H)-carboxylate remained. Several new peaks were shown on LC-MS (Retention time=1.36 min) and one main peak with desired mass was detected. The five batches were combined together for workup. The reaction mixture was poured into H₂O (20 L) slowly and then the mixture was filtered. The filtrate was extracted with MTBE (10 L×5). The organic phase was washed with brine (500 mL), dried over anhydrous Na₂SO₄, concentrated in vacuum to give a residue. The residue was purified by column chromatography (SiO₂, Petroleum ether: Ethyl acetate=3:1 to 1:1, PE:EtOAc=1:1, Rf_{P1}=0.23) to give tert-butyl 9-cyano-2,3-dihydropyrido[3,4-f][1,4]oxazepine-4(5H)-carboxylate (720 g, 2.45 mol, 80.7% yield, 93.7% purity) was obtained as an off-white solid.

¹H NMR: 400 MHz CDCl₃ δ 8.62 (br s, 1H), 8.45 (br s, 1H), 4.40-4.70 (m, 3H), 3.84-3.91 (m, 2H), 1.34-1.46 (m, 9H).

### Procedure for Preparation of Compound 6:

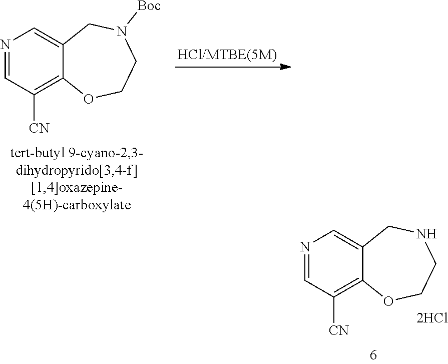 [SMILES: CC(C)(C)OC(=O)N1CCOc2c(C#N)cncc2C1 ; N#Cc1cncc2c1OCCNC2]

Four reactions were carried out in parallel. To a mixture of tert-butyl 9-cyano-2,3-dihydropyrido[3,4-f][1,4]oxazepine-4(5H)-carboxylate (180 g, 654 mmol, 1 eq) in MTBE (1500 mL) was added HCl/MTBE (5 M, 700 mL) drop-wise at 25° C. under N₂. The mixture was stirred at 25° C. for 2 hr. LC-MS showed no tert-butyl 9-cyano-2,3-dihydropyrido[3,4-f][1,4]oxazepine-4(5H)-carboxylate remained. Several new peaks were shown on LC-MS (Retention time=0.46 min) and one main peak with desired mass was detected. The four batches were combined together for workup. The solid was collected by filtration to give compound 6 (620 g, 2.48 mol, 95% yield, 99.4% purity, 2HCl) as an off-white solid.

¹H NMR: 400 MHz DMSO-d6 δ 10.17 (br s, 2H), 8.89 (s, 1H), 8.74 (s, 1H), 4.54-4.74 (m, 2H), 4.54 (s, 2H), 3.60 (s, 2H).

### Procedure for preparation of 4-(3,3-difluoro-2,2-dimethylpropanoyl)-2,3,4,5-tetrahydropyrido[3,4-f][1,4]oxazepine-9-carbonitrile

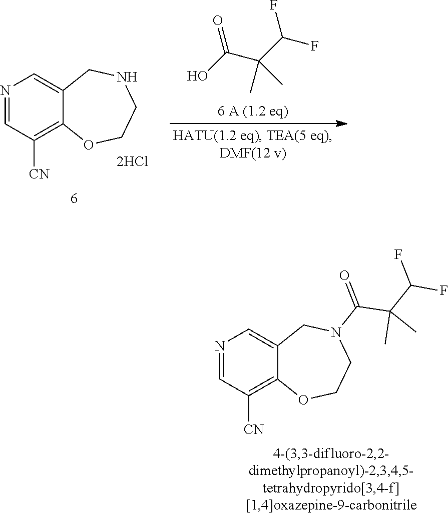

Two reactions were carried out in parallel. To a solution of compound 6A (50.1 g, 363 mmol, 1.2 eq), Et₃N (153 g, 1.51 mol, 210 mL, 5 eq) and compound 6 (75 g, 302 mmol, 1 eq, 2HCl) in DMF (750 mL) was added HATU (138 g, 363 mmol, 1.2 eq) in portions at 0° C. under N₂ atmosphere. The mixture was stirred at 25° C. for 2 hr under N₂ atmosphere. LC-MS showed that no compound 6 remained. Several new peaks were shown on LC-MS (Retention time=1.18 min) and one main peak with desired mass was detected. The two batches were combined together for workup. The reaction mixture was added water (2 L) at 0° C., and the aqueous was extracted with EtOAc 3 L (1 L×3). The combined organic layers were washed with brine (500 mL×3), dried over Na₂SO₄, filtered and concentrated under reduced pressure to give a residue. The crude mixture was dissolved in EtOAc (2 L), and added the Pd-removal silica gel (10 g). The mixture was stirred at 25° C. for 2 hr, then filtered and concentrated under reduced pressure to give a residue. The residue was purified by column chromatography (SiO₂, Heptane:Ethyl acetate=5:1 to 1:1). Then the crude product (180 g) was added EtOAc (200 mL) and the mixture was heated at reflux to provide a clear solution. The solution was filtered under vacuum. The resulting mixture was added n-heptane (100 mL) drop-wise and stirred at 25° C. for 2 hr. Then white solid had crystallized. The white solid was collected by filtration to give 4-(3,3-difluoro-2,2-dimethylpropanoyl)-2,3,4,5-tetrahydropyrido[3,4-f][1,4]oxazepine-9-carbonitrile (85 g, 287.28 mmol, 47.52% yield, 99.8% purity) as a white solid.

¹H NMR: 400 MHZ DMSO-d₆ δ 8.72 (s, 1H), 8.67 (s, 1H), 6.22 (t, J=56.4 Hz, 1H), 4.84 (br s, 2H), 4.73 (t, J=5.2 Hz, 2H), 4.02 (br s, 2H), 1.26 (s, 6H).

The following abbreviations may be relevant for this application.

| | |
|---|---|
| ACN or MeCN: | acetonitrile; |
| CAN: | ceric ammonium nitrate; |
| CPME: | cyclopentyl methyl ether; |
| DCM: | dichloromethane; |
| DMSO: | dimethylsulfoxide; |
| DMAc: | N,N-Dimethylacetamide; |
| DSC: | differential scanning calorimetry; |
| DVS: | dynamic vapor sorption; |
| Et: | ethyl; |
| EtOAc: | ethyl acetate; |
| EtOH: | ethanol; |
| equiv or eq.: | equivalents; |
| FaSSIF: | fasted state simulated intestinal fluid; |
| FeSSIF: | fed state simulated intestinal fluid; |
| FTIR: | Fourier transform infrared; |
| h or hr: | hour; |
| hrs: | hours; |
| HPLC: | high-performance liquid chromatography; |
| IPA: | isopropyl alcohol; |
| IPAc: | isopropyl acetate; |
| KCl: | potassium chloride; |
| LC-MS or LCMS or LC/MS: | liquid chromatography-mass spectrometry; |
| LiCl: | lithium chloride; |
| M: | molar; |
| Me: | methyl; |
| MeOH: | methanol; |
| MeOAc: | methyl acetate; |
| Mg(NO₃)₂: | magnesium nitrate; |
| MIBK: | methyl isobutyl ketone; |
| MTBE: | methyl tert-butyl ether; |
| mins or min: | minutes; |
| N₂: | nitrogen; |
| n-PrOAc: | n-propyl acetate; |
| NMR: | nuclear magnetic resonance; |
| RH: | relative humidity; |
| rt or RT: | room temperature; |
| SCXRD: | single crystal x-ray diffraction; |
| SGF: | simulated gastric fluid; |
| TFA: | trifluoroacetic acid; |
| TGA: | thermogravimetric analysis; |
| THF: | tetrahydrofuran; |
| 2-MeTHF: | 2-methyltetrahydroguran; |
| vol: | volume; |
| w/w: | weight ratio; and |
| XRPD: | X-ray powder diffraction. |

### Instruments and Methods

XRPD. For XRPD analysis, PANalytical Empyrean and X' Pert3 X-ray powder diffractometer were used. The XRPD parameters used are listed in Table A.

TABLE A
Parameters for XRPD test

| Parameters | Empyrean | X′ Pert3 | X′ Pert3 |
|---|---|---|---|
| X-Ray | Cu, Kα; | Cu, Kα; | Cu, Kα; |
| wavelength | Kα1 (Å): 1.540598 | Kα1 (Å): 1.540598 | Kα1 (Å): 1.540598 |
| | Kα2 (Å): 1.544426 | Kα2 (Å): 1.544426 | Kα2 (Å): 1.544426 |
| | intensity ratio | intensity ratio | intensity ratio Kα2/Kα1: |
| | Kα2/Kα1: 0.50 | Kα2/Kα1: 0.50 | 0.50 |
| X-Ray tube | 45 kV, 40 mA | 45 kV, 40 mA | 45 kV, 40 mA |
| setting | | | |
| Divergence | Automatic | 1/8° | 1/8° |
| slit | | | |
| Scan mode | Continuous | Continuous | Continuous |
| Scan range | 3°~40° | 3°~40° | 3°~40° |
| (2θ/°) | | | |
| Step size | 0.0167° | 0.0263° | 0.0263° |
| (2θ/°) | | | |
| Scan step | 17.780 | 46.665 | 39.525 |
| time (s) | | | |
| Test time (s) | About 5 mins 30 s | About 5 mins | 4 mins 27 s |

TGA and DSC. TGA data were collected using a TA Q500/Q5000 TGA from TA Instruments. DSC was performed using a TA Q200/Q2000 DSC from TA Instruments. Detailed parameters used are listed in Table B.

TABLE B
Parameters for TGA and DSC test

| Parameters | TGA | DSC |
|---|---|---|
| Method | Ramp | Ramp |
| Sample pan | Aluminum, open | Aluminum, crimped/open |
| Temperature | RT-desired | 25° C.-desired |
| | temperature | temperature |
| Heating rate | 10° C./min | 10° C./min |
| Purge gas | N₂ | N₂ |

DVS. DVS was measured via a SMS (Surface Measurement Systems) DVS Intrinsic. The relative humidity at 25° C. were calibrated against deliquescence point of LiCl, Mg(NO₃)₂ and KCl. Parameters for DVS test were listed in Table C.

TABLE C
Parameters for DVS test

| Parameters | DVS |
|---|---|
| Temperature | 25° C. |
| Sample size | 10~20 mg |
| Gas and flow rate | N₂, 200 mL/min |
| dm/dt | 0.002%/min |
| Min. dm/dtstabilityduration | 10 min |
| Max. equilibrium time | 180 min |
| RH range | 95% RH-0% RH-95% RH |
| RH step size | 10% (90% RH-0% RH-90% RH) |
| | 5% (95% RH-90% RH and 90% RH-95% |
| | RH) |

HPLC. Agilent HPLC was utilized and detailed chromatographic conditions for purity and solubility measurement are listed in Table D.

TABLE D
Chromatographic conditions and parameters for purity/solubility test

| Parameters | Agilent 1260 DAD Detector | |
|---|---|---|
| Column | Phenomenex Gemini C18, | |
| | 150 × 4.6 mm, 3 μm | |
| Mobile phase | A: 0.037% TFA in Water | |
| | B: 0.018% TFA in Acetonitrile | |
| Gradient table | Time (min) | % B |
| | 0.00 | 10 |
| | 0.10 | 10 |
| | 7.00 | 80 |
| | 10.00 | 100 |
| | 10.01 | 10 |
| | 15.00 | 10 |
| Run time | 15.0 | min |
| Post time | 0.0 | min |
| Flow rate | 0.8 | mL/min |
| Injection volume | 5 | μL |
| Detector wavelength | UV at 220 nm | |
| Column temperature | 40° | C. |

| | |
|---|---|
| Sampler temperature | RT |
| Diluent | Acetonitrile/Water (1:1) |

LC-MS. Shimadzu LC-MS was utilized and detailed conditions for measurement are listed in Table E.

TABLE E
Conditions and parameters for LC-MS test

| Parameters | Shimadzu-LC-MS 2020 | |
|---|---|---|
| Column | Sepax BR-C18 4.6*50 mm, 3 μm | |
| Mobile Phrase | A: 0.1% FA in Water | |
| | B: Acetonitrile | |
| Gradient table | Time (min) | % B |
| | 0.00 | 20 |
| | 0.20 | 20 |
| | 2.00 | 80 |
| | 4.80 | 80 |
| | 5.00 | 20 |
| | 5.50 | 20 |
| Run time | 5.50 | min |
| Flow rate | 1.0 | mL/min |
| Injection volume | 0.4 | μL |
| Detector wavelength | UV at 220/254 nm | |
| Column temperature | 40° | C. |

| | |
|---|---|
| Sampler temperature | RT |
| Ion source for MS | ESI |

PLM. PLM images were captured using Axio Lab A1 upright microscope with ProgRes® CT3 camera at RT.

pKa. The pKa was measured by a Sirius pKa log P/D tester (model: T3) with a UV detector (UV metric method) using MeOH as solvent.

Compound (1), made as described herein, was characterized by XRPD, TGA, DSC, PLM, DVS and HPLC purity.

As displayed in FIG. 1, XRPD revealed that the sample was crystalline and thus named as Form A. Peaks identified in FIG. 1 include those listed in Table 1.

TABLE 1
XRPD Peak list of Form A

| Pos. | d- | Rel. |
|---|---|---|
| [°2Th.] | spacing | Int. |
| (±0.2) | [Å] | [%] |
| 10.0 | 8.84 | 10.1 |
| 14.3 | 6.17 | 100.0 |
| 14.8 | 5.99 | 9.5 |
| 16.4 | 5.40 | 13.4 |
| 18.2 | 4.9 | 9.7 |
| 20.1 | 4.4 | 40.5 |
| 21.0 | 4.2 | 25.2 |
| 21.6 | 4.1 | 44.9 |
| 22.8 | 3.9 | 55.8 |
| 23.5 | 3.8 | 33.0 |
| 28.1 | 3.2 | 27.0 |
| 29.8 | 3.0 | 11.9 |

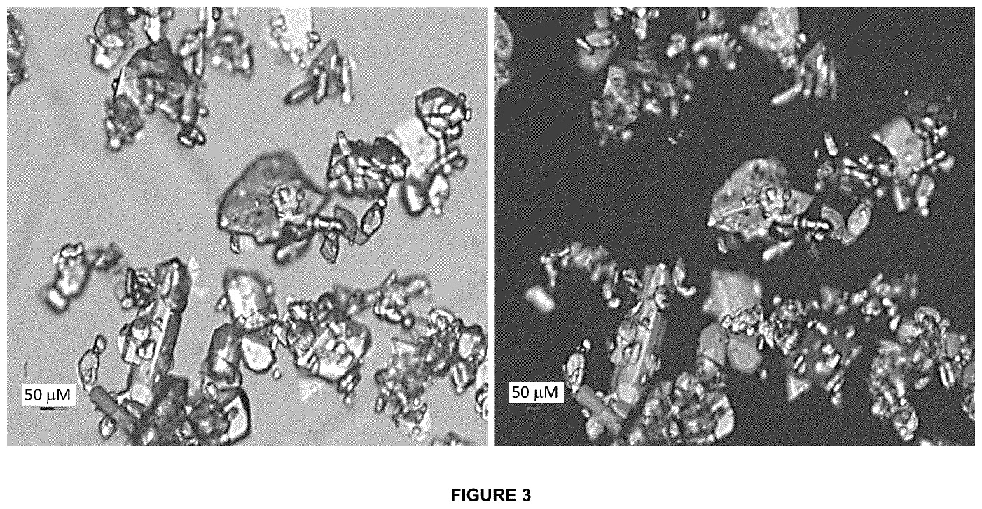

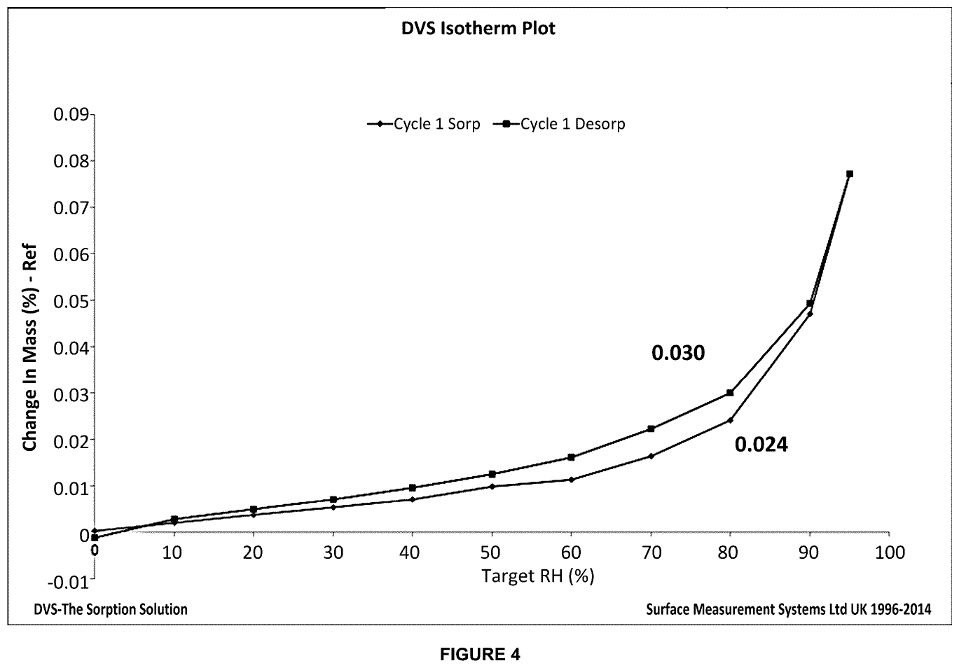

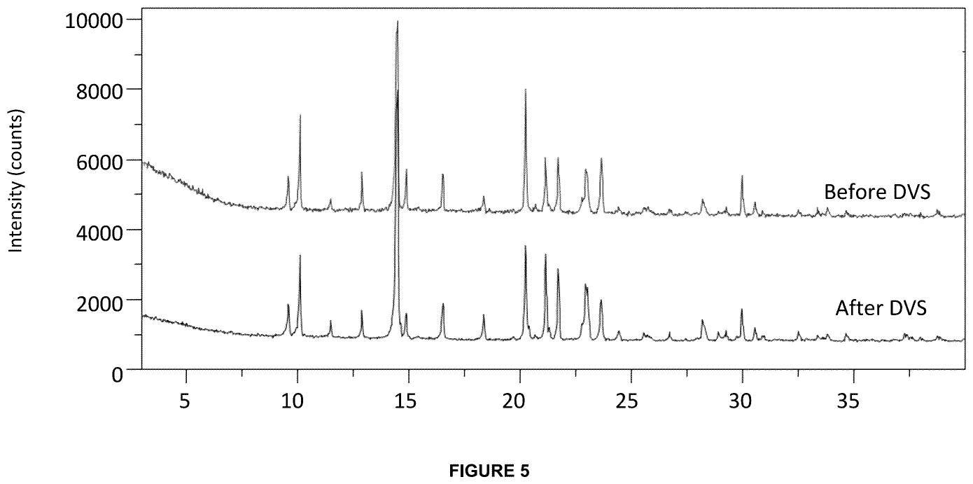

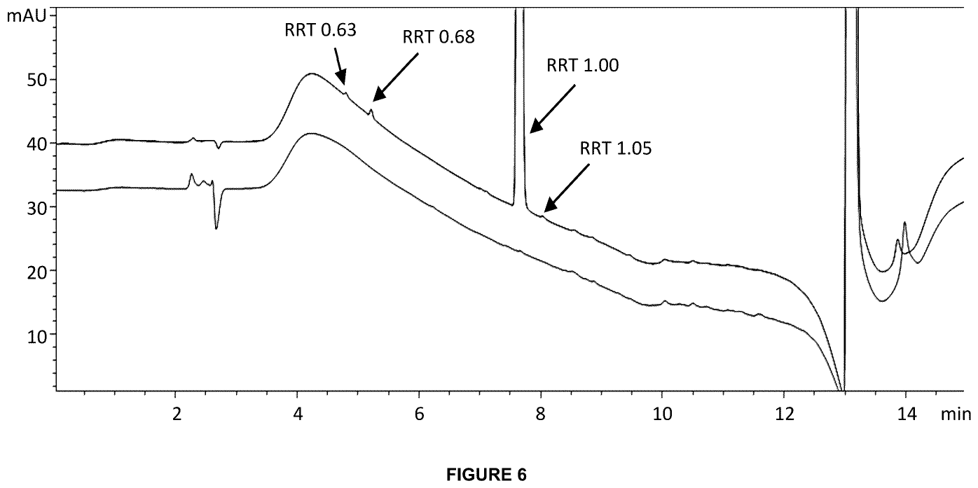

TGA and DSC data are shown in FIG. 2. A weight loss of 0.9% was observed up to 120° C. on the TGA curve.¹ The DSC result exhibited one sharp endotherm at 128.5° C. (onset temperature). Considering the low TGA weight loss and single sharp DSC endotherm, Form A was postulated to be an anhydrate. The PLM images shown in FIG. 3 indicated that irregular-shaped crystals with particle size of 50˜200 μm were observed. The DVS plot (FIG. 4) indicated that a water uptake of 0.024% was observed at 25 C.°/80% RH. XRPD overlay in FIG. 5 indicated that no form change was observed after DVS test. The HPLC purity of starting material was measured as 99.78 area % (see chromatogram of FIG. 6) and the impurity summary is listed in Table 2. ¹ Description of the TGA data: The TGA value in the Figure shows a 0.9% weight loss. However Form A has been prepared with much lower volatile content (0.1% or lower).

TABLE 2
Impurity summary of
Compound (1) starting material

| #Peak | RRT | Area % |
|---|---|---|
| 1 | 0.63 | 0.05 |
| 2 | 0.68 | 0.12 |
| 3 | 1.00 | 99.78 |
| 4 | 1.05 | 0.06 |

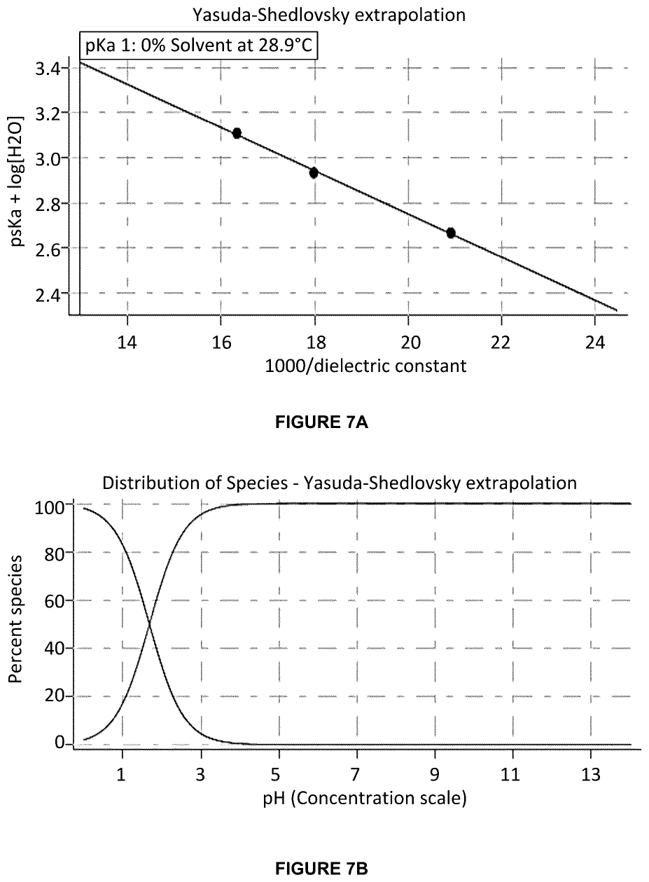

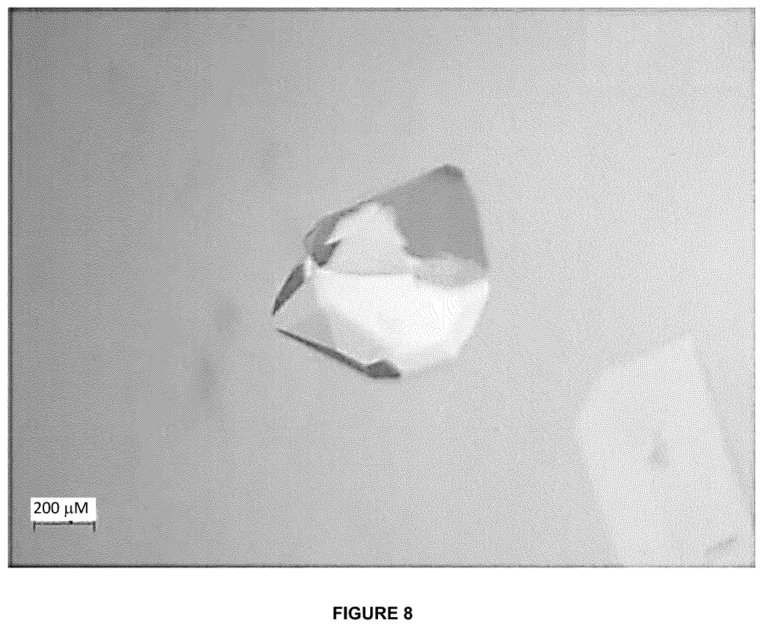

In addition, the pKa value of Compound (1) starting material was measured to be 1.68 by a Sirius pKa log P/D tester (model: T3) with a UV detector (UV metric method) using MeOH as solvent. The pKa value should be taken as reference because the effective pH range of UV metric method is pH 2-12. Detailed results of pKa measurement are listed in Table 3 and FIGS. 7A and 7B. FIG. 7A shows the Yasuda-Shedlovsky extrapolation and FIG. 7B shows the distribution of species.

TABLE 3
pKa measurement results for Compound (1)

| Extrapolation | | | | | | Ionic | |
|---|---|---|---|---|---|---|---|
| type | pKa | % SD | Intercept | Slope | R₂ | strength | Temperature |
| Yasuda- | 1.68 | ±0.02 | 4.67 | −95.7995 | 0.9983 | 0.176M | 28.9° C. |
| Shedlovsky | | | | | | | |

### Example 2. Solid Form Screening

A total of 96 solid form screening experiments were performed using different crystallization or solid transformation methods. The results are summarized in Table 4 and the experiment details are set forth below. Only one crystal form of Compound (1), Form A, was observed from screening.

TABLE 4
Summary of polymorph screening experiments

| | No. of | |
|---|---|---|
| Method | Experiment | Result |
| Anti-solvent addition | 12 | Form A |
| Reverse anti-solvent addition | 8 | Form A |
| Slow evaporation | 13 | Form A |
| Slow cooling | 8 | Form A |
| Slurry at RT | 13 | Form A |
| Slurry at 50° C./70° C.* | 8 | Form A |
| Slurry Cycling (5~50° C.) | 10 | Form A |
| Vapor-solid diffusion | 8 | Form A |
| Vapor-solution diffusion | 8 | Form A |
| Polymer induced crystallization | 8 | Form A |
| Total | 96 | Form A |

*The slurry experiments were performed at 50° C. for 2 days, followed by slurrying at 70° C. for 3 days.

### Example 2.1. Anti-Solvent Addition

A total of 12 anti-solvent addition experiments were carried out. About 15 mg of Compound (1) starting material was dissolved in 0.1-0.5 mL solvent to obtain a clear solution and the solution was magnetically stirred (˜1000 rpm) followed by addition of 0.1 mL anti-solvent per step till precipitate appeared or the total amount of anti-solvent reached 10 mL. The obtained precipitate was isolated for XRPD analysis. Results, as summarized in Table 5, indicate that only Form A was generated.

TABLE 5
Summary of anti-solvent addition experiments

| Experiment ID | Solvent | Anti-solvent | Solid Form |
|---|---|---|---|
| 1* | MeOH | H₂O | Form A |
| 2* | Acetone | | Form A |
| 3* | THF | | Form A |
| 4* | 1,4-Dioxane | | Form A |
| 5 | DCM | n-Heptane | Form A |
| 6 | n-PrOAc | | Form A |
| 7 | MIBK | | Form A |
| 8 | CHCl₃ | Cyclohexane | Form A |
| 9 | MeOAc | | Form A |
| 10 | 2-MeTHF | | Form A |
| 11** | Dimethyl | m-Xylene | Form A |
| | carbonate | | |
| 12** | ACN | | Form A |

*Solid was obtained after stirring at 5° C.
Clear solution was obtained after anti-solvent addition and stirring at 5° C., which was transferred to evaporate at RT before vacuum drying at 50° C.

### Example 2.2. Reverse Anti-Solvent Addition

Reverse anti-solvent addition experiments were conducted under 8 conditions. Approximately 15 mg of Compound (1) starting material was dissolved in 0.1-0.3 mL of each solvent to get a clear solution. This solution was added dropwise into a glass vial containing 5 mL of each antisolvent at RT. The precipitate was isolated for XRPD analysis. Results, as summarized in Table 6, showed that only Form A was generated.

TABLE 6
Summary of reverse anti-solvent addition experiments

| | | | Solid |
|---|---|---|---|
| Experiment # | Solvent | Anti-solvent | Form |
| 1* | DMSO | H₂O | Form A |
| 2* | DMAC | | Form A |
| 3** | EtOAc | | Form A |
| 4 | CHCl3 | n-Heptane | Form A |
| 5 | IPAC | | Form A |
| 6 | DCM | Cyclohexane | Form A |
| 7 | Acetone | | Form A |
| 8** | NMP | m-Xylenes | Form A |

*Solid was obtained after stirring at 5° C.
**Clear solution was obtained after stirring at 5° C., and then transferred to RT for evaporation.

### Example 2.3. Slow Evaporation

Slow evaporation experiments were performed under 13 conditions. Briefly, ˜15 mg of Compound (1) starting material was dissolved in 0.2˜2.0 mL of solvent in a 3-mL glass vial. If not dissolved completely, suspensions were filtered using a PTFE membrane (pore size of 0.45 μm) and the filtrates would be used instead for the follow-up steps. The visually clear solutions were subjected to evaporation at RT with vials sealed by Parafilm® (poked with 6 pinholes). The solids were isolated for XRPD analysis, and the results, as summarized in Table 7, indicated that only Form A was obtained.

TABLE 7
Summary of slow evaporation experiments

| Experiment # | Solvent (v:v) | Solid Form |
|---|---|---|
| 1 | MeOH | Form A |
| 2 | Acetone | Form A |
| 3 | EtOAc | Form A |
| 4 | CPME | Form A |
| 5 | 2-MeTHF | Form A |
| 6 | ACN | Form A |
| 7 | DCM | Form A |
| 8 | 1,4-Dioxane | Form A |
| 9 | Dimethyl carbonate | Form A |
| 10 | THF | Form A |
| 11 | IPA | Form A |
| 12 | CHCl₃/MTBE (1:4) | Form A |
| 13 | MeOH/Toluene (1:4) | Form A |

### Example 2.4. Slow Cooling

Slow cooling experiments were conducted in 8 solvent systems. About 15 mg of Compound (1) starting material was suspended in 0.7 mL of solvent in an HPLC vial at RT. The suspension was then heated to 50° C., equilibrated for about 2 hours and filtered to a new vial using a PTFE membrane (pore size of 0.45 μm) if not completely dissolved. Filtrates were slowly cooled down to 5° C. at a rate of 0.1° C./min. The obtained solids were kept isothermal at 5° C. before isolated for XRPD analysis. Clear solutions were evaporated to dryness at RT and then solids were tested by XRPD. Results, summarized in Table 8, indicated Form A was obtained.

TABLE 8
Summary of slow cooling experiments

| | | Solid |
|---|---|---|
| Experiment # | Solvent (v:v) | Form |
| 1* | CPME | Form A |
| 2 | IPA | Form A |
| 3 | Toluene | Form A |
| 4* | EtOH | Form A |
| 5** | MTBE/Cyclohexane (1:1) | Form A |
| 6** | Acetone/n-Heptane (1:9) | Form A |
| 7 | EtOH/m-Xylene (1:1) | Form A |
| 8* | MeOH/H₂O (1:1) | Form A |

*Solid was obtained after stirring at 5° C.
**Clear solution was obtained after stirring at 5° C. and −20° C., and then transferred to RT for evaporation.

### Example 2.5. Slurry at RT

Slurry conversion experiments were conducted at RT in 13 different solvent systems. ˜15 mg of Compound (1) starting material was suspended in 0.5 mL of solvent in an HPLC vial. After the suspension was stirred magnetically (˜700 rpm) for about 7 days at RT, the remaining solids were isolated for XRPD analysis. The results, as summarized in Table 9, showed that only Form A was generated.

TABLE 9
Summary of slurry conversion experiments at RT

| Experiment # | Solvent (v:v) | Solid Form |
|---|---|---|
| 1 | Cyclohexane | Form A |
| 2 | H₂O | Form A |
| 3 | n-Heptane | Form A |
| 4 | Toluene | Form A |
| 5 | CPME | Form A |
| 6 | MTBE | Form A |
| 7 | NMP/H₂O (1:9) | Form A |
| 8 | IPA/H₂O (0.97:0.03, a_{w}~0.3) | Form A |
| 9 | IPA/H₂O (0.92:0.08, a_{w}~0.6) | Form A |
| 10 | IPA/H₂O (0.77:0.23, a_{w}~0.9) | Form A |
| 11 | CHCl₃/m-Xylene (1:9) | Form A |
| 12 | MIBK/Cyclohexane (1:9) | Form A |
| 13 | IPAc/n-Heptane (1:9) | Form A |

### Example 2.6. Slurry at 50° C./70° C.

Slurry conversion experiments were also conducted at 50° C. in 8 different solvent systems. About 15 mg of Compound (1) starting material was suspended in 0.5 mL of solvent in an HPLC vial. After the suspension was magnetically stirred (˜700 rpm) for about 2 days at 50° C., the remaining solids were isolated for XRPD analysis and only Form A was generated. The samples were then transferred to stir at 70° C. for another 3 days, the remaining solids were isolated for XRPD analysis. Results, as summarized in Table 10, indicate that only Form A was generated.

TABLE 10
Summary of slurry conversion experiments at 50° C./70° C.

| | | Solid Form | Solid Form |
|---|---|---|---|
| Experiment # | Solvent (v:v) | (50° C.) | (70° C.) |
| 1 | H₂O | Form A | Form A |
| 2 | m-Xylene | Form A | Form A |
| 3* | Toluene | Form A | Form A |
| 4 | n-Heptane | Form A | Form A |
| 5 | ACN/H₂O (1:9) | Form A | Form A |
| 6 | IPA/Cyclohexane (1:9) | Form A | Form A |
| 7 | Anisole/n-Heptane (1:9) | Form A | Form A |
| 8* | EtOAc/m-Xylene (1:9) | Form A | Form A |

*Clear solution was obtained after 50° C. stirring, then ~20 mg starting material was further added.

### Example 2.7. Slurry Cycling (50-5° C.)

Slurry cycling (50-5° C.) experiments were conducted in 10 different solvent systems. About 15 mg of Compound (1) starting material was suspended in 0.5 mL of solvent in an HPLC vial. The suspensions were magnetically stirred (˜700 rpm) at 50° C. for 2 hours and then slowly cooled down to 5° C. at a rate of 0.1° C./min. The obtained solids were kept isothermal at 5° C. after cycled between 50° C. and 5° C. for 3 times. Solids were isolated for XRPD analysis. The results, as summarized in Table 11, indicate that only Form A was generated.

TABLE 11
Summary of slurry cycling (50-5° C.) experiments

| Experiment # | Solvent (v:v) | Solid Form |
|---|---|---|
| 1 | IPA | Form A |
| 2 | MTBE | Form A |
| 3 | Cyclohexane | Form A |
| 4 | CPME | Form A |
| 5 | Toluene | Form A |
| 6 | MeOH/H₂O (1:4) | Form A |
| 7 | Acetone/H₂O (1:4) | Form A |
| 8 | MTBE/n-Heptane (1:9) | Form A |
| 9 | Dimethyl | Form A |
| | carbonate/Cyclohexane (1:9) | |
| 10 | THF/m-Xylenes (1:9) | Form A |

### Example 2.8. Vapor Solid Diffusion

Eight vapor-solid diffusion experiments were performed using different solvents. About 15 mg of Compound (1) starting material was weighed into a 3-mL glass vial. This 3-mL vial was then placed into a 20-mL vial with 4 mL of solvents. The 20-mL vial was sealed with a cap and kept at RT for 7 days. The solids were isolated for XRPD analysis. The results, as summarized in Table 12, indicate that only Form A was generated.

TABLE 12
Summary of vapor-solid diffusion experiments

| Experiment # | Solvent | Solid Form |
|---|---|---|
| 1 | H₂O | Form A |
| 2 | EtOH | Form A |
| 3 | IPA | Form A |
| 4 | EtOAc | Form A |
| 5* | THF | Form A |
| 6 | 1,4-Dioxane | Form A |
| 7 | DMSO | Form A |
| 8 | Toluene | Form A |

*Clear solution was obtained, and then transferred to RT for evaporation.

### Example 2.9. Vapor-Solution Diffusion

Eight vapor-solution diffusion experiments were conducted. Approximate 15 mg of Compound (1) starting material was dissolved in 0.3-1.5 mL of appropriate solvent to obtain a clear solution in a 3-mL vial. This solution was then placed into a 20-mL vial with 4 mL of volatile solvents. The 20-mL vial was sealed with a cap and kept at RT allowing sufficient time for organic vapor to interact with the solution. Clear solution was obtained after 12 days and transferred to evaporate at RT. The solids were isolated for XRPD analysis. The results, as summarized in Table 13, indicate that only Form A was generated.

TABLE 13
Summary of vapor-solution diffusion experiments

| Experiment # | Solvent | Anti-solvent | Solid Form |
|---|---|---|---|
| 1 | THF | H₂O | Form A |
| 2 | ACN | | Form A |
| 3 | Acetone | | Form A |
| 4 | MeOAc | Cyclohexane | Form A |
| 5 | EtOH | | Form A |
| 6 | 2-MeTHF | n-Heptane | Form A |
| 7 | IPAC | | Form A |
| 8 | 1,4-Dioxane | m-Xylene | Form A |

### Example 2.10. Polymer Induced Crystallization

Polymer induced crystallization experiments were performed with two sets of polymer mixtures in 8 different solvent systems. Approximate 15 mg of Compound (1) starting material was dissolved in 0.5-1.5 mL of solvent in a 3-mL glass vial. About 1 mg of polymer mixture was added into the 3-mL glass vial. The resulting solutions were subjected to evaporation at RT with vials sealed by Parafilm® (poked with 3 pinholes) for slow evaporation. The solids were isolated for XRPD analysis. The results, as summarized in Table 14, indicate that only Form A was generated.

TABLE 14
Summary of polymer induced crystallization experiments

| | | | Solid |
|---|---|---|---|
| Experiment ID | Solvent (v:v) | Polymer | Form |
| 1 | IPA | Polymer | Form A |
| 2 | Toluene | mixture A | Form A |
| 3 | MeOAc | | Form A |
| 4 | n-PrOAc/EtOH (1:1) | | Form A |
| 5 | MTBE | Polymer | Form A |
| 6 | CHCl₃ | mixture B | Form A |
| 7 | Acetone | | Form A |
| 8 | MIBK/Toluene (1:1) | | Form A |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1).
Polymer mixture B: polycaprolactone (PCL), polyethylene glycol (PEG), polymethyl methacrylate (PMMA) sodium alginate (SA), and hydroxyethyl cellulose (HEC) (mass ratio of 1:1:1:1:1).

### Example 3. Single Crystal Data for Compound (1) Form A

Block-like single crystals of Compound (1) Form A used for SCXRD characterization were crystallized from MeOH/toluene (1:4, v/v) solvent mixture by slow evaporation method. The experimental details are elaborated further below.

First, 14.7 mg of Compound (1) starting material was weighed into a 3-mL glass vial followed by addition of 1.5 mL MeOH/toluene (1:4, v/v) solvent mixture. After being oscillated on a vortex and ultrasonically shaken to accelerate dissolution, the suspension was then filtered through PTFE filter membrane (0.45 μm) and disposable syringe into a new 3-mL glass vial. The vial was then covered by seal membrane (Parafilm®) with six pinholes on it for slow evaporation at RT. After ˜10 days, block-like single crystals (CP ID: 814904-09-A13) were obtained as shown in FIG. 8.

A suitable single crystal with good diffraction quality was selected out from the block-like crystal samples and was wrapped with Paratone-N (an oil based cryoprotectant). The crystal was mounted on a mylar loop in a random orientation and immersed in a stream of nitrogen at 175 K. Preliminary examination and data collection were performed on a Bruker D8 VENTURE diffractometer (Mo/Kα radiation, λ=0.71073 Å) and analyzed with the APEX3 software package.

Cell parameters and an orientation matrix for data collection were retrieved and refined (least-squares refinement) by SAINT (Bruker, V8.37A, after 2013) software using the setting angles of 9951 reflections in the range 2.333°<θ<27.040°. The data were collected to a maximum diffraction angle (θ) of 27.549° at 175K. The data set was 99.80% complete out to 27.549° in θ, having a Mean I/σ of 20.9 and D min (Mo) of 0.77 Å.

Frames were integrated with SAINT (Bruker, V8.37A, after 2013). A total of 36148 reflections were collected, of which 3204 were unique. Lorentz and polarization corrections were applied to the data. A multi-scan absorption correction was performed using SADABS-2014/5 (Bruker, 2014/5) wR₂(int) was 0.0981 before and 0.0709 after correction. The absorption coefficient μ of this material is 0.114 mm⁻¹ at this wavelength (λ=0.71073 Å) and the minimum and maximum transmissions are 0.7025 and 0.7456. Intensities of equivalent reflections were averaged. The agreement factor for the averaging was 6.17% based on intensity.

The structure was solved in the space group P21/c by Intrinsic Phasing using the ShelXT structure solution program, as set forth in Sheldrick, G. M. “A short history of SHELX,” Acta Crystallogr. Sect. A (2008) A64, 112-122, and refined by Least Squares using version 2017/1 of ShelXL (Sheldrick, Acta Crystallogr. (2015) C71, 3-8) refinement package contained in OLEX2 (Dolomanov et al. (2009), J. Appl. Cryst. 42, 339-341). All non-hydrogen atoms were refined anisotropically. The positions of hydrogen atoms were refined freely according to the Fourier Map.

The structure of the crystal was determined successfully. The crystal system is monoclinic and the space group is P21/c. The cell parameters are: a=9.3375(10) Å, b=8.5568(9) Å, c=17.6497(19) Å, α=90°, β=98.412(3)°, γ=90°, V=1395.0(3) Å³. The formula weight is 295.29 g·mol⁻¹ with Z=4, resulting in the calculated density of 1.406 g·cm⁻³. Further crystallographic data and the refinement parameters are listed in Table 15.

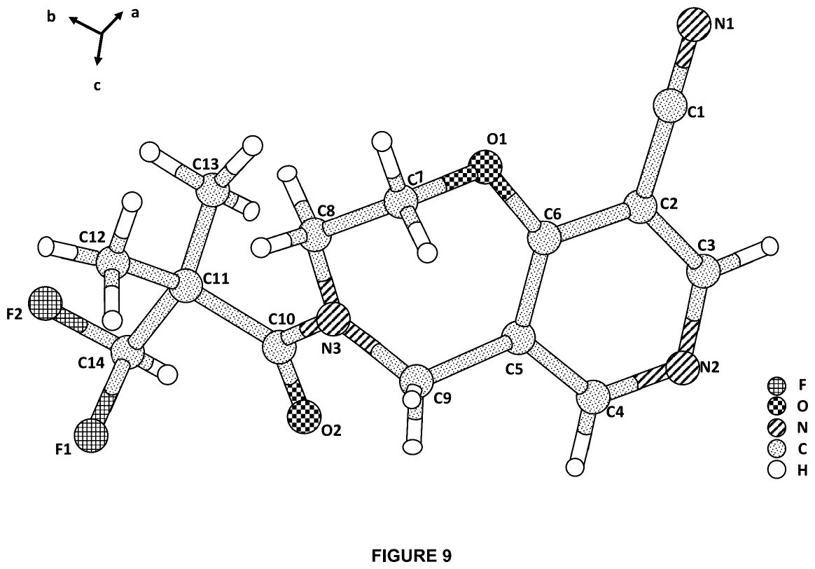

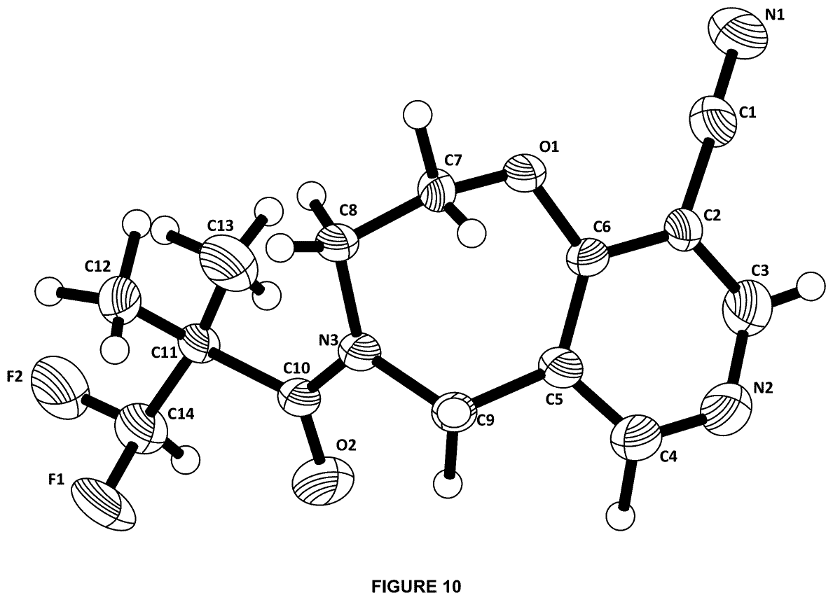

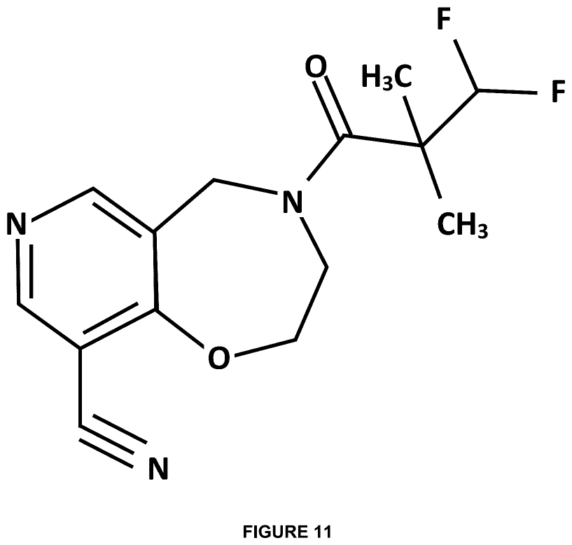

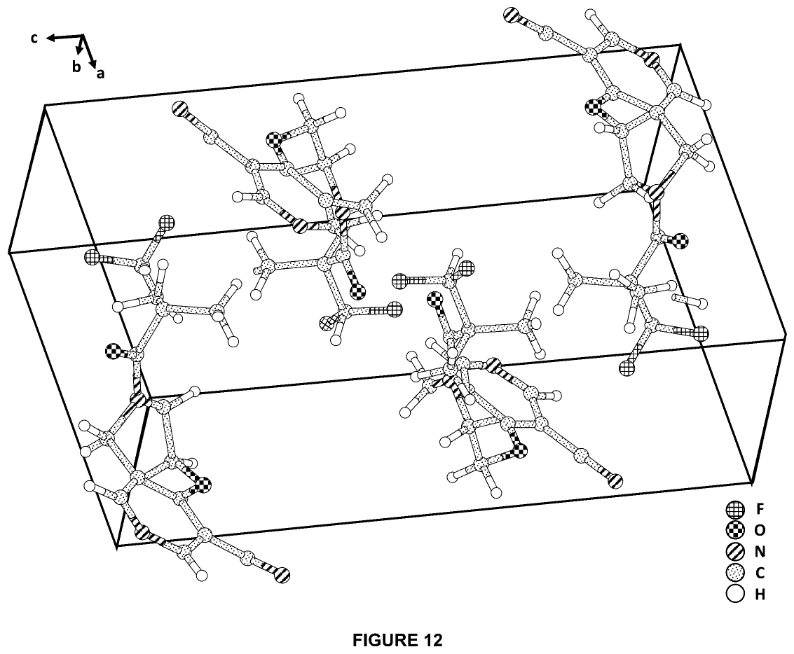

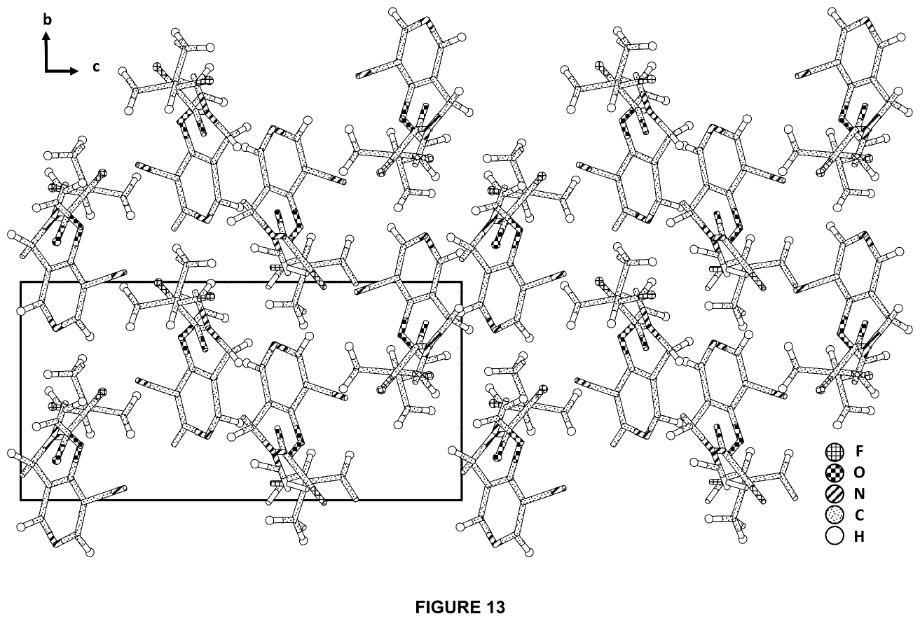

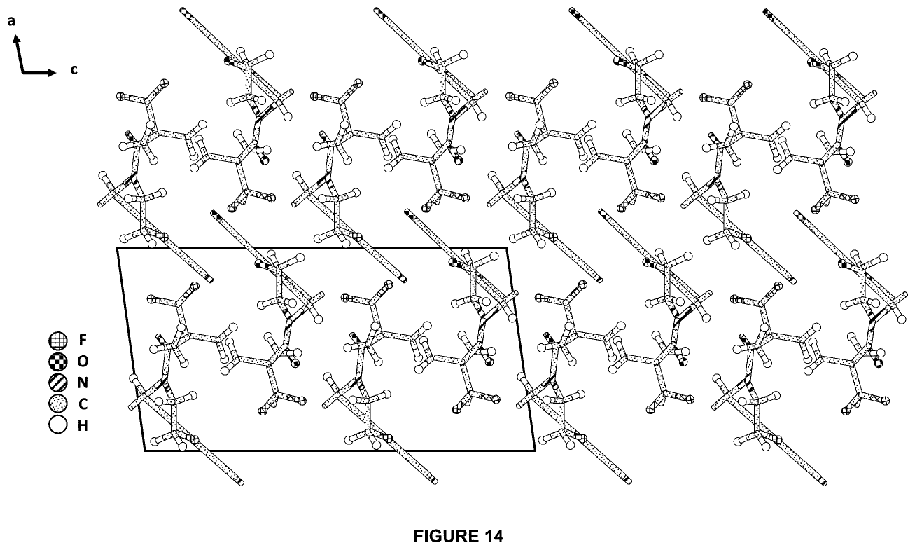

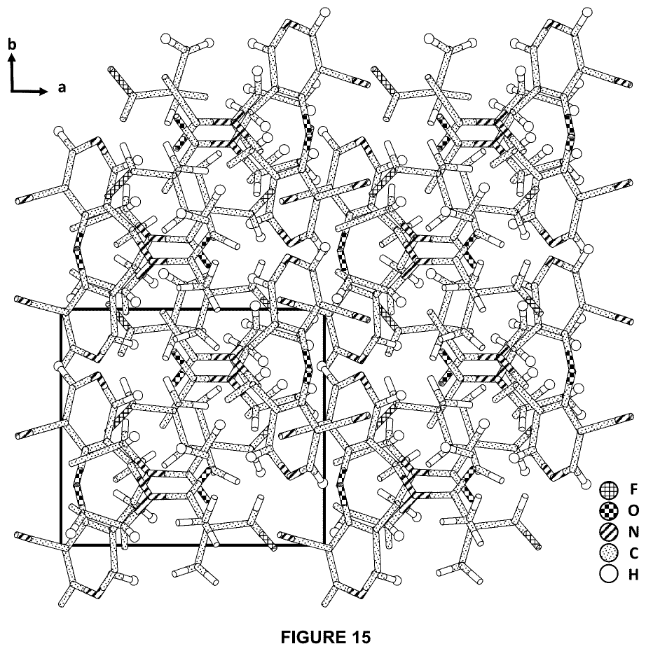

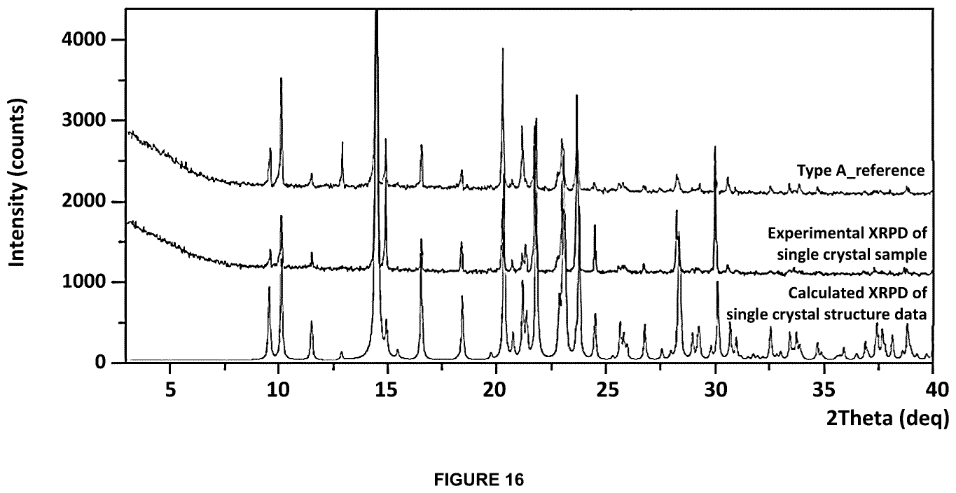

As shown in FIG. 9, the asymmetric unit of the single crystal structure is comprised of only one Compound (1) molecule, indicating the crystal is an anhydrate of Compound (1). The thermal ellipsoids drawing of the Compound (1) molecule in the crystal lattice is shown in FIG. 10. The single crystal structure determination confirmed that the structure of Compound (1) is consistent with the proposed chemical structure as shown in FIG. 11. The unit cell of the single crystal is shown in FIG. 12. The packing diagrams viewed along the crystallographic a-axis, b-axis, c-axis are shown in FIG. 13, FIG. 14, and FIG. 15, respectively.

The calculated XRPD pattern was generated for Cu radiation using Mercury⁴ program and the atomic coordinates, space group, and unit cell parameters from the single crystal structure. The calculated XRPD generated from the single crystal structure data and the experimental XRPD pattern of the single crystal sample are consistent with Compound (1) Form A reference as shown in Table 15.

TABLE 15
Crystallographic data and refinement parameters

| Identification code | Compound (1) Form A | |
|---|---|---|
| Empirical formula | C₁₄H₁₅F₂N₃O₂ | |
| Formula weight | 295.29 | |
| Temperature | 175 | K |
| Wavelength | Mo/Kα (λ = 0.71073 Å) | |
| Crystal system, space group | monoclinic, P2₁/c | |
| Unit cell dimensions | a = 9.3375(10) Å | |
| | b = 8.5568(9) Å | |
| | c = 17.6497(19) Å | |
| | α = 90° | |
| | β = 98.412(3)º | |
| | γ = 90° | |
| Volume | 1395.0(3) Å3 | |
| Z, Calculated density | 4, 1.406 | g/cm³ |
| Absorption coefficient | 0.114 | mm⁻¹ |

| | |
|---|---|
| F(000) | 616.0 |
| Crystal size | 0.7 × 0.6 × 0.5 mm³ |
| 2 Theta range for data collection | 5.934° to 55.098° |
| Limiting indices | −12 ≤ h ≤ 12 |
| | −11 ≤ k ≤ 11 |
| | −22 ≤ 1 ≤ 22 |
| Reflections collected/ | 36148/3204 [Rᵢₙₜ = 0.0617, |
| Independent reflections | R_{sigma} = 0.0334] |
| Refinement method | Full-matrix least-squares on F² |
| Data/restraints/parameters | 3204/0/250 |
| Goodness-of-fit on F² | 1.045 |
| Final R indices [I ≥ 2sigma(I)] | R₁ = 0.0456, wR₂ = 0.1009 |
| Final R indices [all data] | R₁ = 0.0697, wR₂ =0.1116 |
| Largest diff. peak and hole | 0.21/−0.25 e.Å⁻³ |

### Example 4. Compound (1) Form A Evaluation

### Example 4.1—Physical and Chemical Stability

To evaluate the physical and chemical stability, Compound (1) Form A was stored in 3 conditions (40° C./75% RH; 25° C./60% RH; and 60° C.) for one and four weeks. All samples were characterized using XRPD and HPLC purity, with the results summarized in Table 16.

TABLE 16
Stability evaluation summary of Form A

| | | | | | Purity |
|---|---|---|---|---|---|
| | | | | | VS. |
| Initial | Time | | | Final | initial |
| Form | point | Condition | Description | Form | (%) |
| Form A | Initial | NA | White powder | Form A | NA |
| | 1 week | 40° C./75% RH | White powder | Form A | 100.0 |
| | | 25° C./60% RH | White powder | Form A | 100.0 |
| | | 60° C. | White powder | Form A | 100.0 |
| | 4 weeks | 40° C./75% RH | White powder | Form A | 100.0 |
| | | 25° C./60% RH | White powder | Form A | 100.0 |
| | | 60° C. | White powder | Form A | 100.0 |

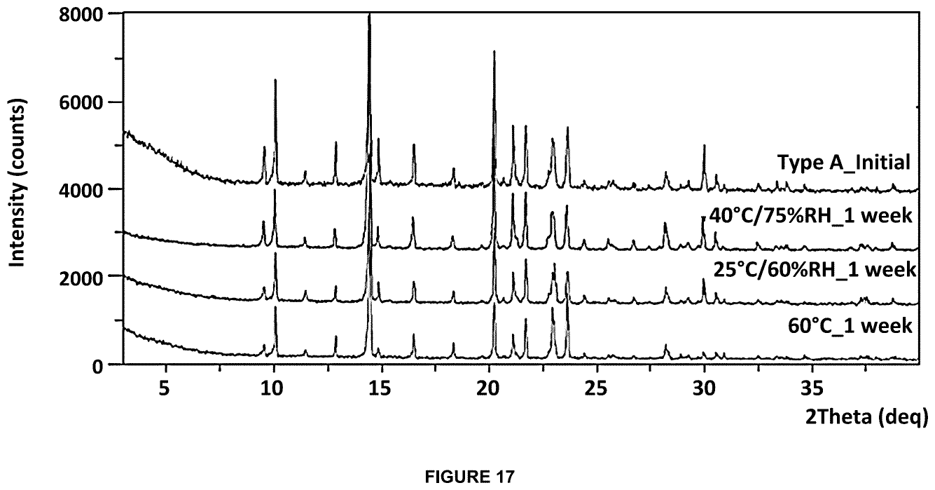

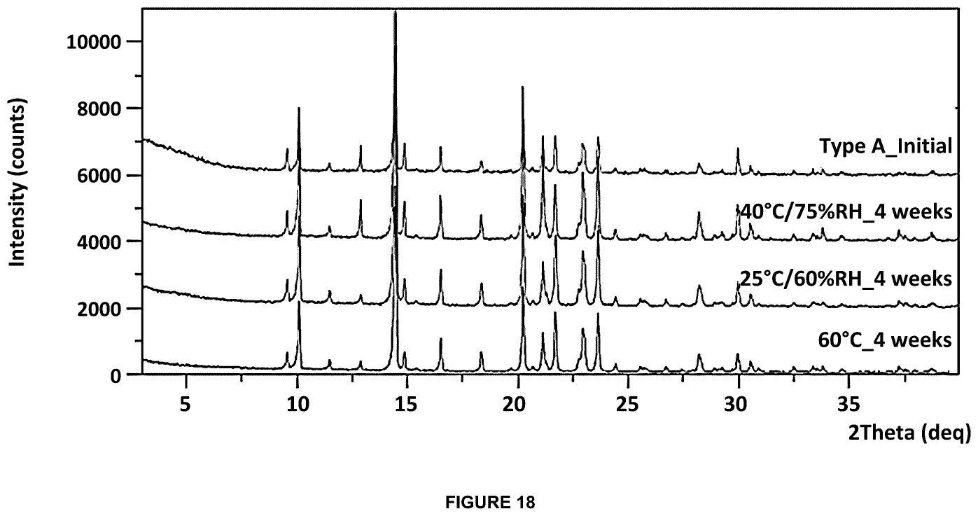

XRPD results from FIG. 17 to FIG. 18 indicated no form change was observed for Form A under all conditions. HPLC result indicated that no obvious HPLC purity change was observed. Detailed impurities of Form A were summarized in Table 17.

TABLE 17
Impurity summary of Form A after stability evaluation

| | | | % Area | |
|---|---|---|---|---|
| | | | | API |
| Initial | Time | | Imp.1 | (RRT |
| Form | point | Condition | (RRT 0.68) | 1.00) |
| Form | Initial | NA | 0.11 | 99.89 |
| A | 1 week | 40° C./75% RH | 0.11 | 99.89 |
| | | 25° C./60% RH | 0.08 | 99.92 |
| | | 60° C. | 0.10 | 99.90 |
| | 4 weeks | 40° C./75% RH | 0.10 | 99.90 |
| | | 25° C./60% RH | 0.10 | 99.90 |
| | | 60° C. | 0.10 | 99.90 |

### Example 4.2. Kinetic Solubility

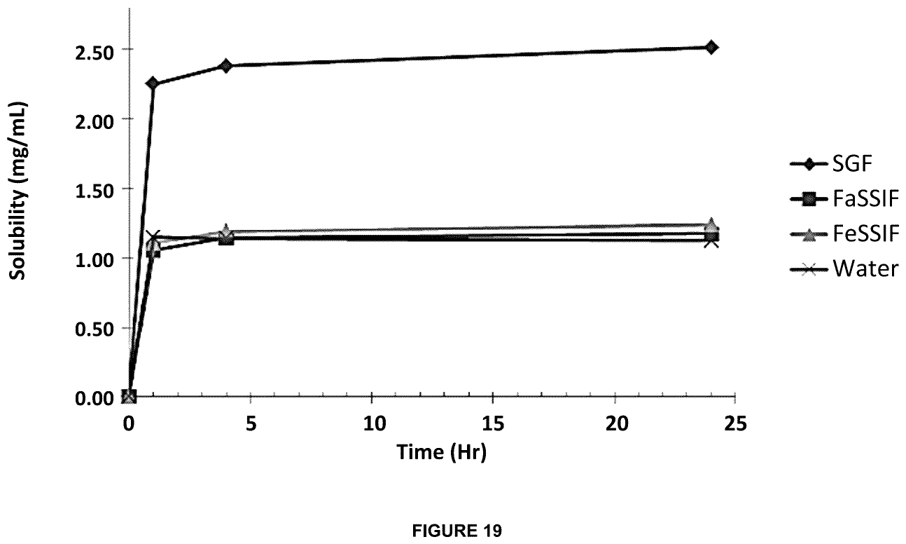

Kinetic solubility of Compound (1) Form A was evaluated in bio-relevant media (SGF, FaSSIF and FeSSIF) and H₂O at 37° C. for 1, 4, 24 hrs. Solids were suspended in FaSSIF, FeSSIF, SGF and H₂O with target conc. of ˜10 mg/mL. The suspensions were agitated on a rolling incubator at 25 rpm (in the incubator set at 37° C.) for 1, 4 and 24 hrs. At each time point, 1 mL of the suspension was pipetted out for centrifugation at 15000 rpm (3 min) and filtration through 0.45 μm membrane to obtain supernatant for HPLC solubility and pH tests, the residual solids were analyzed by XRPD. The solubility data of Form A are summarized in Table 18 and the solubility curves are shown in FIG. 19.

TABLE 18
Summary of kinetic solubility results of Form A

| | | Time | | | | |
|---|---|---|---|---|---|---|
| Initial | | point | Final | Solubility | Obser- | Final |
| Form | Media | (hr) | Form | (mg/mL) | vation | pH |
| Form A | SGF | 1 | Form A | 2.3 | Turbid | 1.8 |
| | (pH 1.8) | 4 | Form A | 2.4 | Turbid | 2.3 |
| | | 24 | Form A | 2.5 | Turbid | 2.2 |
| | FaSSIF | 1 | Form A | 1.1 | Turbid | 6.4 |
| | (pH 6.5) | 4 | Form A | 1.1 | Turbid | 6.6 |
| | | 24 | Form A | 1.2 | Turbid | 6.6 |
| | FeSSIF | 1 | Form A | 1.1 | Turbid | 5.6 |
| | (pH 5.0) | 4 | Form A | 1.2 | Turbid | 5.6 |
| | | 24 | Form A | 1.2 | Turbid | 5.6 |
| | H₂O | 1 | Form A | 1.1 | Turbid | 8.5 |
| | (pH 6.5) | 4 | Form A | 1.1 | Turbid | 8.4 |
| | | 24 | Form A | 1.1 | Turbid | 8.7 |

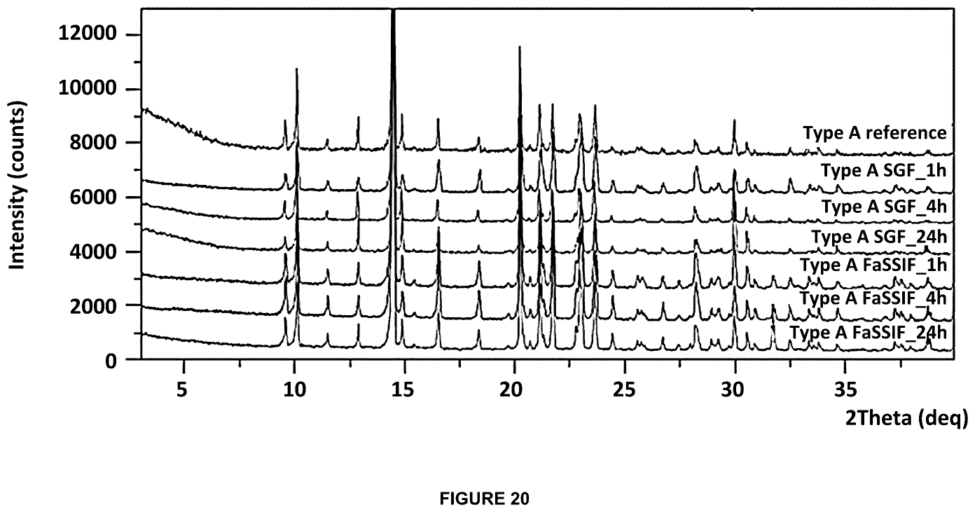

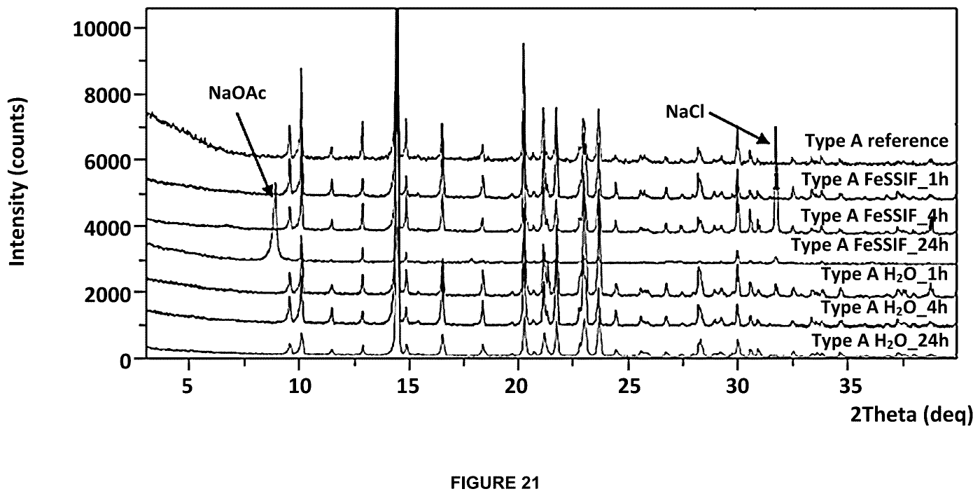

No form change was observed after kinetic solubility test in bio-relevant media or H₂O. The XRPD overlays are displayed in FIG. 20 and FIG. 21.

### Example 4.3. pH Solubility

24-Hrs solubility of Form A was measured in pH buffers (i.e., pH 2.0, 4.0, 6.0, 7.0, 8.0) at RT. Solids were suspended in pH buffers with target conc. of ˜10 mg/mL. The suspensions were stirred (1000 rpm) at 37° C. for 24 hrs, prior to centrifugation at 12000 rpm (2 min) and filtration through 0.45 μm membrane to obtain supernatant for HPLC solubility and pH tests, the residual solids were analyzed by XRPD. Detailed results were summarized in Table 19.

TABLE 19
24-Hrs solubility results summary of Form A in pH buffers

| Experi- | | Final | Solubility | Obser- | Final |
|---|---|---|---|---|---|
| ment # | Media | form | (mg/mL) | vation | pH |
| 1 | pH 2.0 | Form A | 1.8 | Turbid | 2.3 |
| | (50 mM HCl—KCl) | | | | |
| 2 | pH 4.0 | Form A | 0.84 | Turbid | 4.1 |
| | (50 mM Citrate) | | | | |
| 3 | pH 6.0 | Form A | 0.75 | Turbid | 5.9 |
| | (50 mM Citrate) | | | | |
| 4 | pH 7.0 | Form A | 0.90 | Turbid | 6.9 |
| | (50 mM Phosphate) | | | | |
| 5 | pH 8.0 | Form A | 0.81 | Turbid | 7.8 |
| | (50 mM Phosphate) | | | | |

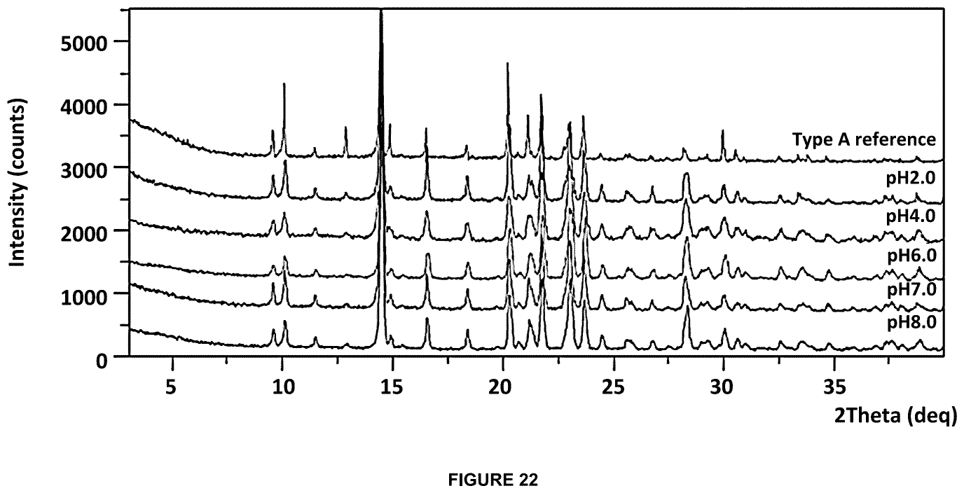

As shown in FIG. 22, no form change was observed for Form A after equilibrium solubility evaluation in pH buffers.

### Example 4.4. Solution Stability Evaluation

Solution stability study was performed in pH 2.0/4.0/6.0/7.0 (24 hrs) and pH 8.0 (24 hrs and 96 hrs) buffers. Solids were dissolved with pH buffers with target conc. of ˜0.5 mg/mL to form clear solutions and stored at 37° C. for 24 hrs or 96 hrs. The stability results are summarized in Table 20 and Table 21.

TABLE 20
Summary of solution stability results in pH buffers

| Experiment | | Time | | Purity vs. |
|---|---|---|---|---|
| # | Media | point | Observation | Initial (%) |
| 1 | pH 2.0 | 24 hrs | Clear | 85.1 |
| | (50 mM HCl—KCl) | | | |
| 2 | pH 4.0 | 24 hrs | Clear | 98.5 |
| | (50 mM Citrate) | | | |
| 3 | pH 6.0 | 24 hrs | Clear | 99.8 |
| | (50 mM Citrate) | | | |
| 4 | pH 7.0 | 24 hrs | Clear | 99.9 |
| | (50 mM Phosphate) | | | |
| 5 | pH 8.0 | 24 hrs | Clear | 100.0 |
| | (50 mM Phosphate) | | | |
| 6 | pH 8.0 | 96 hrs | Clear | 100.0 |
| | (50 mM Phosphate) | | | |

TABLE 21
Impurity summary of solution stability in pH buffers

| | | | % Area | | | | |
|---|---|---|---|---|---|---|---|
| Experiment | | Time | Imp. 1 | Imp. 1 | Imp. 1 | API | Imp. 1 |
| # | Media | point | (RRT 0.66) | (RRT 0.70) | (RRT 0.76) | (RRT 1.00) | (RRT 1.05) |
| 1 | pH 2.0 | 24 hrs | 0.15 | 0.67 | 14.25 | 84.93 | — |
| | (50 mM HCl—KCl) | | | | | | |
| 2 | pH 4.0 | 24 hrs | 0.15 | — | 0.38 | 98.42 | 0.06 |
| | (50 mM Citrate) | | | | | | |
| 3 | pH 6.0 | 24 hrs | 0.13 | — | 0.06 | 99.75 | 0.06 |
| | (50 mM Citrate) | | | | | | |
| 4 | pH 7.0 | 24 hrs | 0.15 | — | — | 99.78 | 0.07 |
| | (50 mM Phosphate) | | | | | | |
| 5 | pH 8.0 | 24 hrs | 0.13 | — | — | 99.81 | 0.06 |
| | (50 mM | | | | | | |
| | Phosphate) | | | | | | |
| 6 | pH 8.0 | 96 hrs | 0.13 | — | — | 99.83 | 0.04 |
| | (50 mM Phosphate) | | | | | | |

—: <0.05 area %

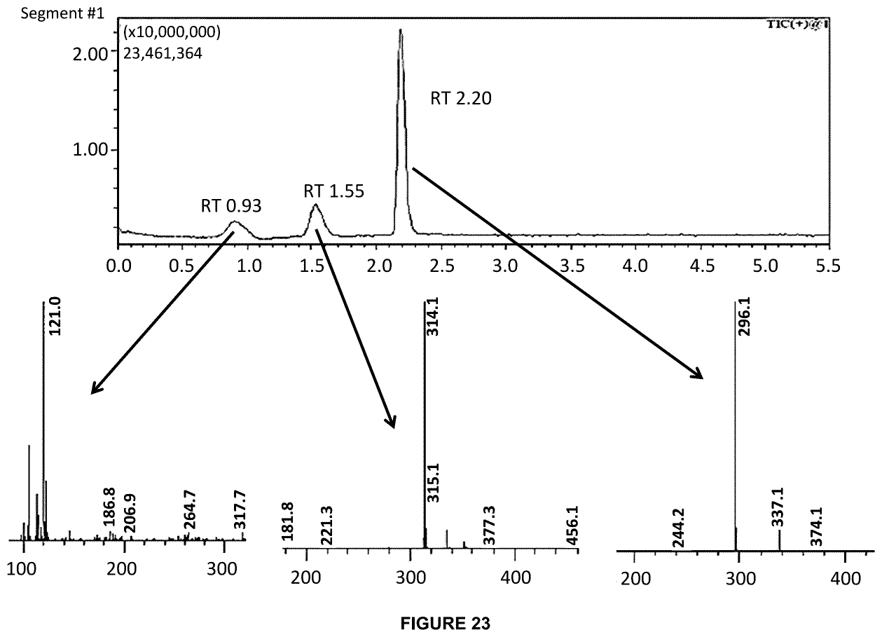

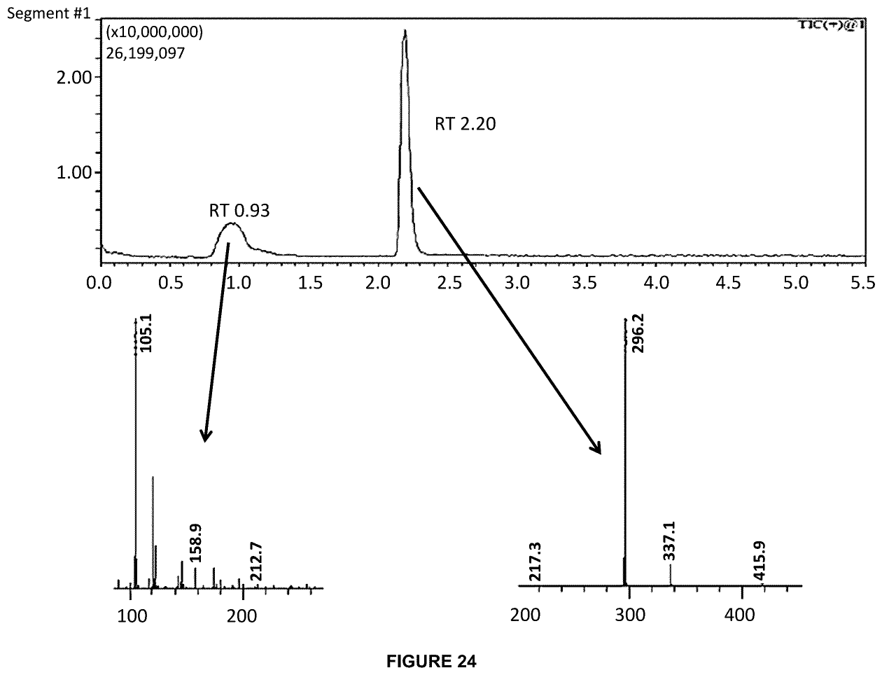

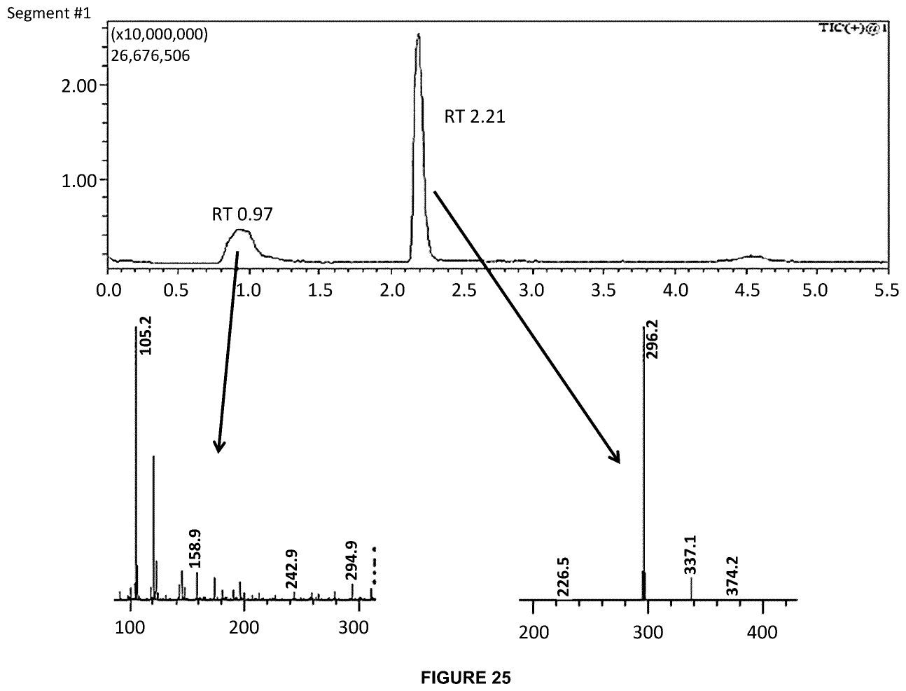

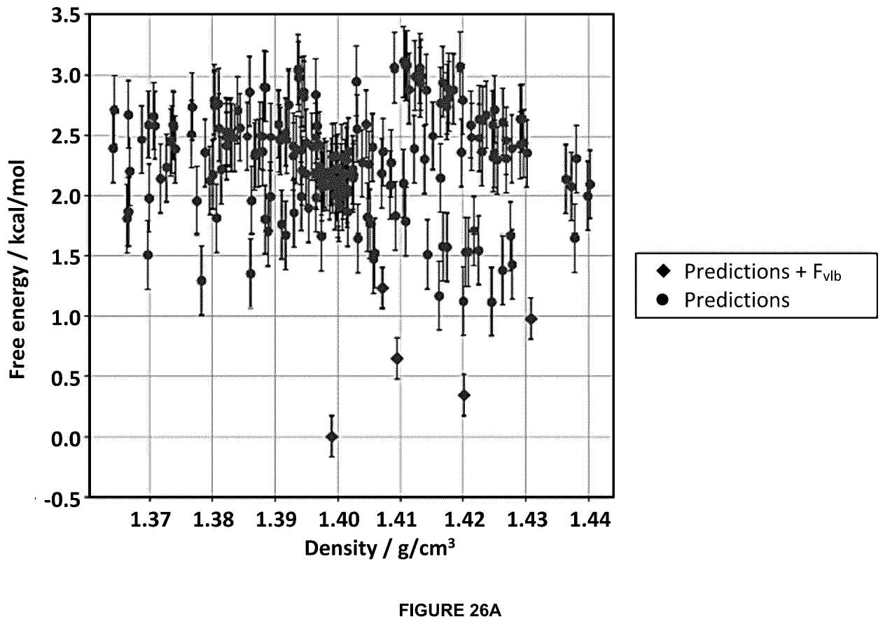

Degradation was observed in pH 2.0 and pH 4.0 buffers. For pH 2.0 and pH 8.0 samples, LC-MS was performed to determine the molecular weight of the impurities. The LC chromatograms and mass spectra are shown in FIGS. 23-25.

### Example 5. In Silico Polymorphism Study of Compound (I)

Hardware. The calculations were carried out on 384 cores of Intel XEON ES processors or equivalent hardware.

Computational details.

### Description of the Compound

Compound (I) contains five flexible torsion angles, including two methyl groups, and one flexible ring. The compound contains no chiral centers. The 2D structure of the compound is shown in FIG. 3.

### Standard Search Space (for Possible Deviations, See Below)

Crystal structures were first generated with one (Z′=1) molecule per asymmetric unit. According to the statistics of the Cambridge Structural Database (CSD), 88.3% of all compounds crystallize with one molecule per asymmetric unit.

The crystal structure generation was carried out in 38 space groups that cover 99.92% of the crystal structures with Z′=1 according to CSD statistics (P1, P−1, P21, C2, Pc, Cc, P2/c, P2₁/c, C2/c, P2₁2₁2, P2₁2₁2₁, C222₁, Pca2₁, Pna2₁, Aba2, Fdd2, lba2, Pcca, Pccn, Pbcn, Pbca, Fddd, P4₁, I4, I4₁, I−4, P42/n, I4₁/a, P4₁2₁2, I4₁cd, P−42₁c, P3₁, R3, R−3, P3₁21, R3c, P6₁, P6₁22). A value of D=1.0 kcal/mol was chosen for the target energy window in which the completeness of the CSP procedure is statistically controlled.

After the Z′=1 structure generation, crystal structures with two molecules per asymmetric unit were constructed from Z′=1 structures by, e.g., unit-cell doubling. This stage is known as the smart Z′=2 CSP.

Crystal structures were also generated with two molecules per asymmetric unit (Z′=2) in a standard search. According to the statistics of the Cambridge Structural Database (CSD), 10.5% of all compounds crystallize with two molecules per asymmetric unit. The Z′=2 case was dealt with in two independent rounds. In the first round, only the space groups P1, P−1 and P2₁ were considered which cover 42.5% of the Z′=2 cases in the CSD. In the second round, the space groups C2, Pc, Cc, P21/c, C2/c, P212121, Pca21, Pna21 were considered. These space groups cover an additional 53.4% of the Z′=2 cases.

Deviations from the standard procedure. None.

CPU time consumption. The tailor-made force field was generated in 4 days. The actual crystal structure prediction took 60 days.

The PBE(0)+MBD energy calculations for 216 structures took 4 days.

The PBE(0)+MBD+Fvib energy calculations for 5 structures took 2.5 days.

TABLE 22
Some numbers from the energy calculations

| | Z′ = 1 | Smart Z′ = 2 | Z′ = 2 part I | Z′ = 2 part II |
|---|---|---|---|---|
| Step 1 | 10,000 | 6538 | 702 | 7825 |
| Step 2 | 194 | 1465 | 179 | 1670 |
| Step 3 | 26 | 161 | 15 | 15 |
| σ(Step 1 → Step 2)¹ | 0.17 | 0.16 | 0.20 | 0.16 |
| σ(Step 2 → Step 3)¹ | 0.003 | 0.005 | 0.002 | 0.002 |
| Step 1 convergence | 99% | 100% | 95% | 95% |
| Step 1 energy window | 4.6σ | 3.8σ | 2.9σ | 2.9σ |

¹kcal/mol/\sqrt{Nₐₜₒₘₛ}

In step 4, the energies of all 216 step 3 structures were computed with PBE(0)+MBD.

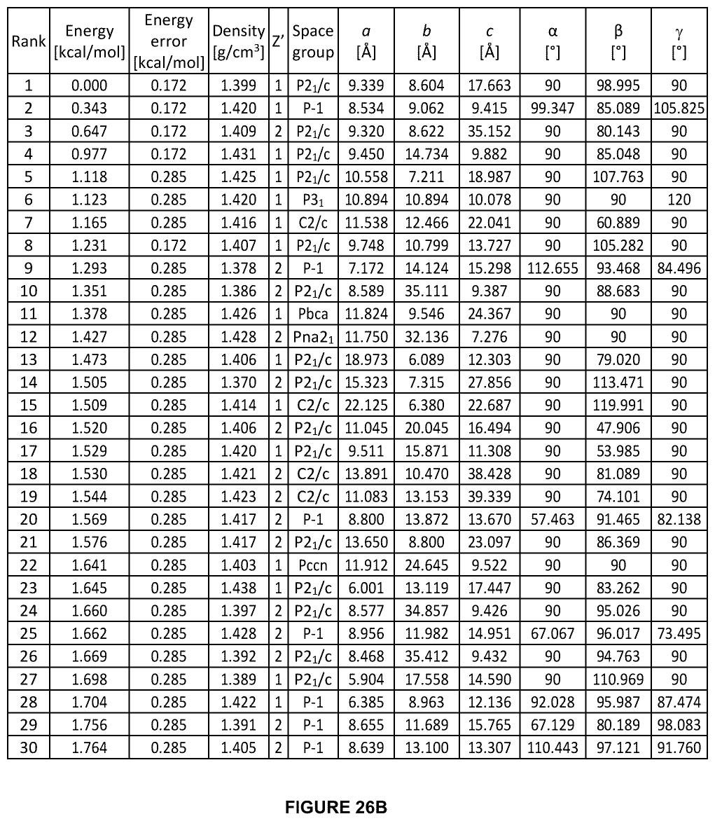

Predicted structures. The 30 most stable OFT-optimized crystal structures with step 4 (free) energies calculated at the PBE(0)+MBD(+Fvib) level is shown in FIG. 26B, and FIG. 26A shows the free energy landscape. For the properties of the 30 most stable predicted structures from step 4, the Fvib correction was computed at 298.15 K.

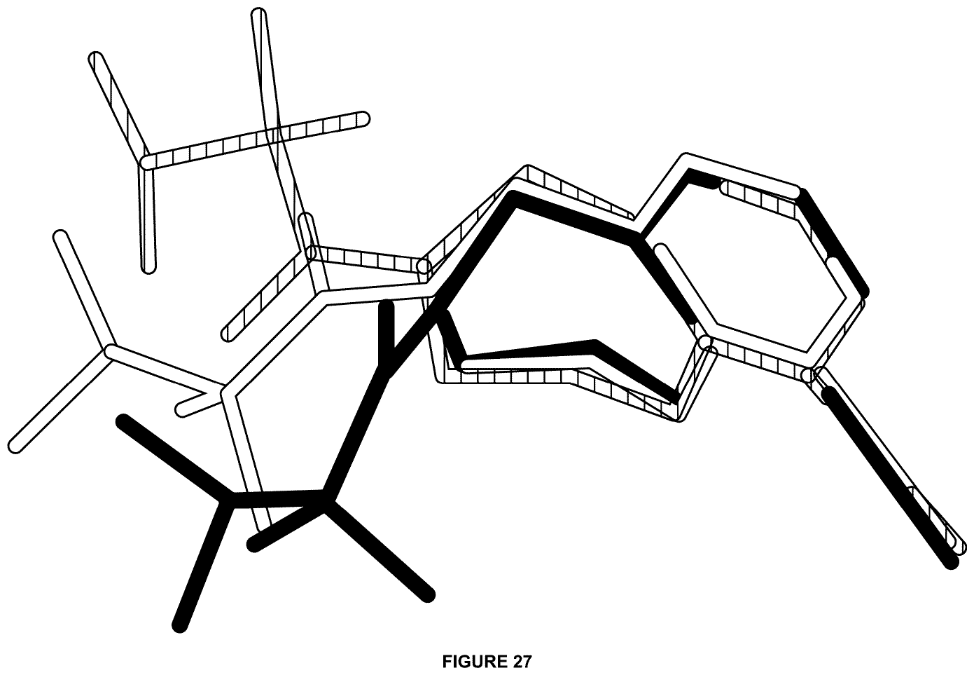

There are no voids greater than 20 Å3/Z in any of the predicted structures. The compound contains no hydrogen-bond donors. Although the molecule is fairly rigid, its shape can change considerably between crystal structures, as shown in FIG. 27.

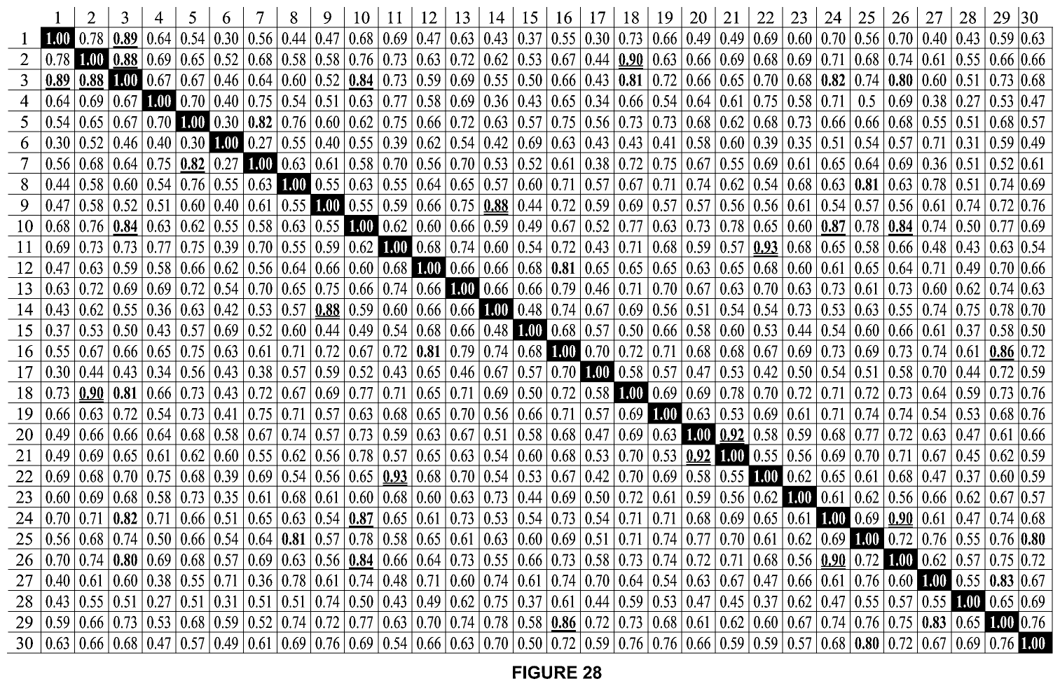

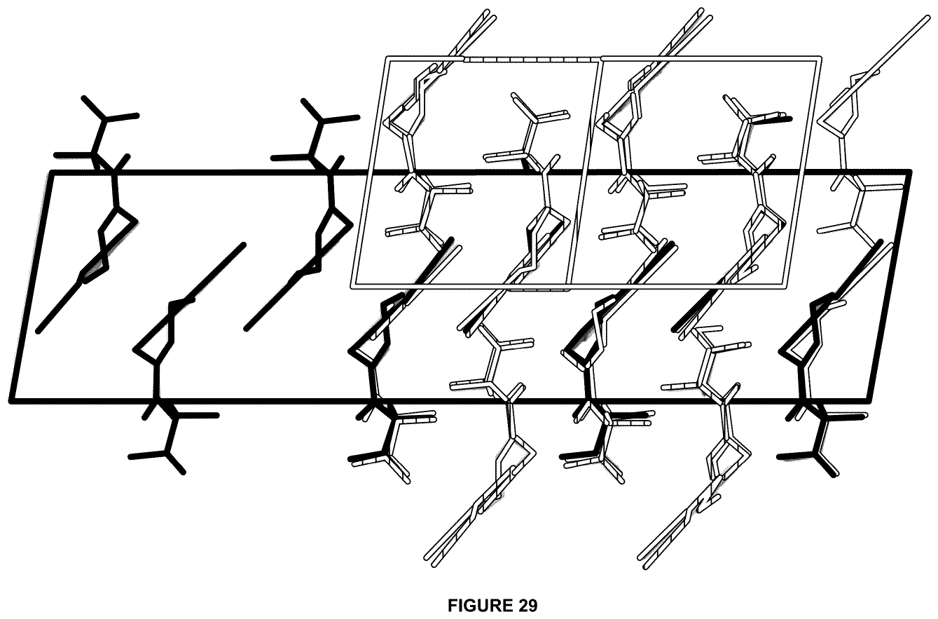

A similarity matrix was calculated for the first 30 structures as the normalized cross-correlation between the simulated powder diffraction patterns. This is graphically represented in FIG. 28 in which the similarity matrix is shown with values from 0.8 to 1.0. Ranks 1, 2 and 3 show some similarity; indeed in projection they can be overlaid (see FIG. 29). In three dimensions, ranks 1, 2 and 3 are similar but different.

Comparison with Compound (I), Form a Single-Crystal Data

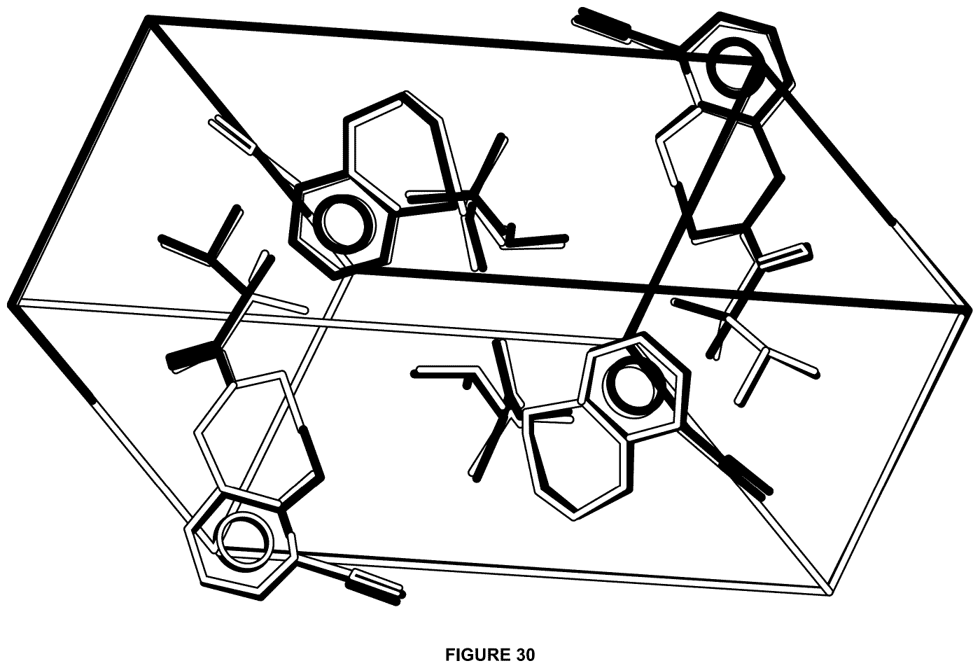

Form A matches the predicted rank 1 structure. FIG. 30 shows the overlay of form A with rank 1.

### Free Energy Landscape in the Context of the Experimental Structures

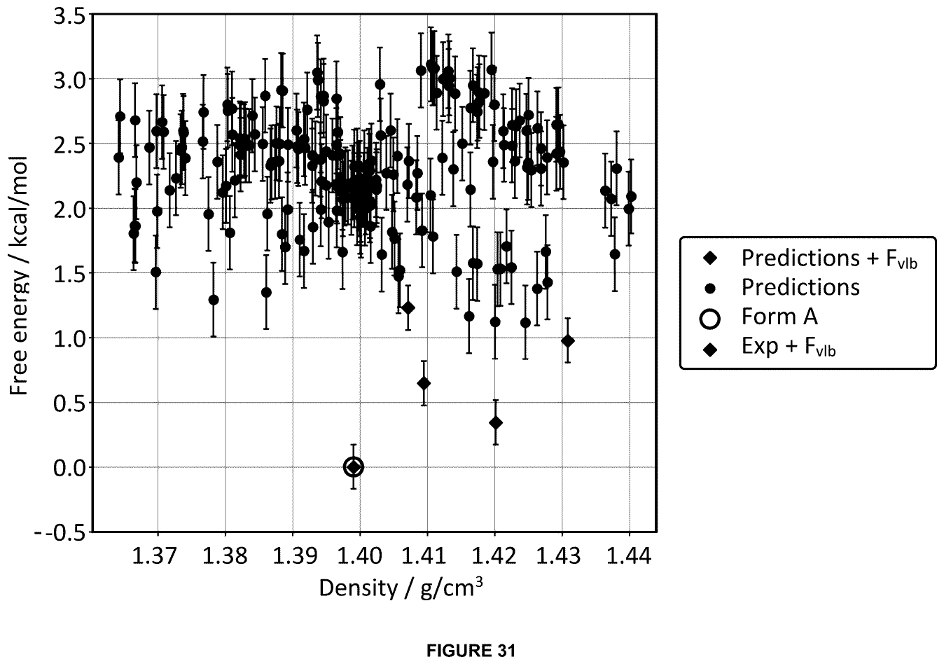

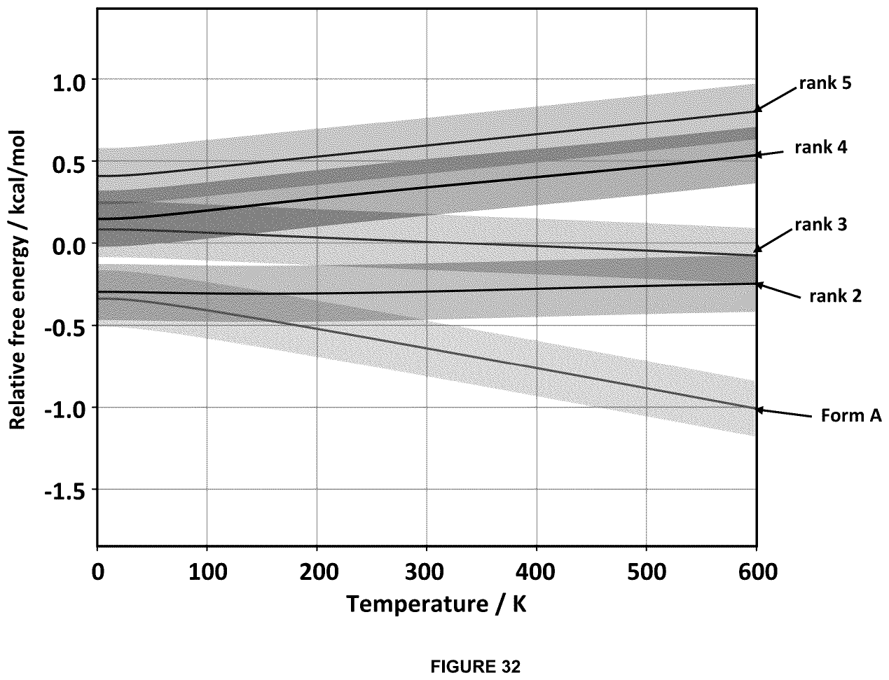

As seen in FIGS. 31-32, the most stable predicted structure (rank 1) matches Form A. Ranks 1, 2 and 3 are very similar, and from a kinetics point of view, if one of these could crystallize then all of them could crystallize. We therefore interpret the fact that rank 1 (=Form A) crystallized as meaning that rank 1 (=Form A) is the thermodynamically most stable structure; this is in agreement with the calculations. The first rank that is not similar to Form A is rank 4, 0.977 kcal/mol less stable than Form A. The error bar is 0.172 kcal/mol, so rank 4 is more than 5 s away from Form A.

As noted herein, Compound 1 can be prepared according to the methods and schemes described in, e.g., U.S. Pat. No. 11,203,600. It can also be made as set forth below.

### Example 6. Amorphous Compound 1

### Example 6.1. Purely Amorphous Form

A purely amorphous form of 4-(3,3-difluoro-2,2-dimethylpropanoyl)-2,3,4,5-tetrahydropyrido[3,4-f][1,4]oxazepine-9-carbonitrile was made by placing a small sample of the compound into a 2 mL glass vial and heating it at 135° C. for about 1 min until the compound melts to an oil. Thereafter the vial was flash cooled in a dry-ice acetone bath, and the resulting product was immediately (within 5 minutes) analyzed by XRPD as described herein.

### Example 6.2. Substantially Amorphous Form

A substantially amorphous form of 4-(3,3-difluoro-2,2-dimethylpropanoyl)-2,3,4,5-tetrahydropyrido[3,4-f][1,4]oxazepine-9-carbonitrile was made as follows:

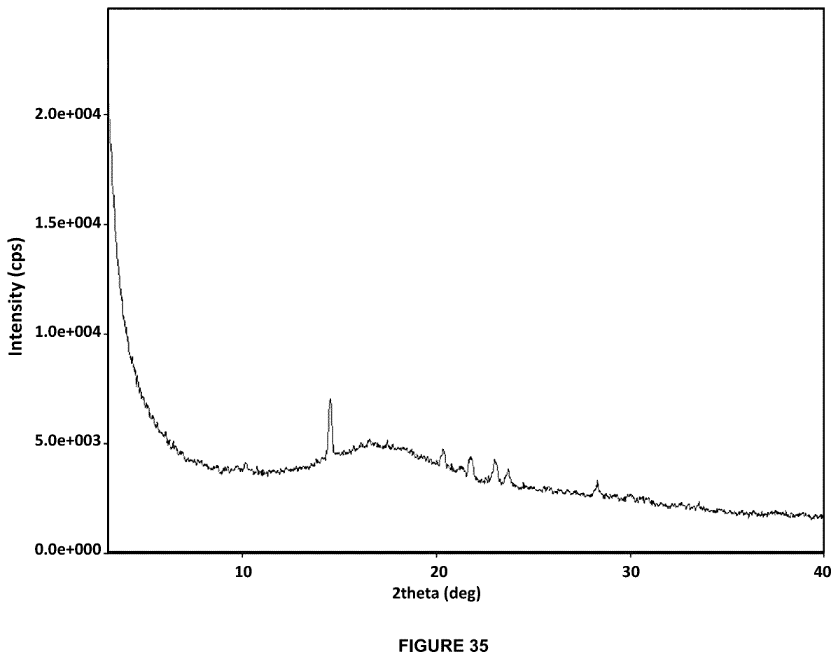

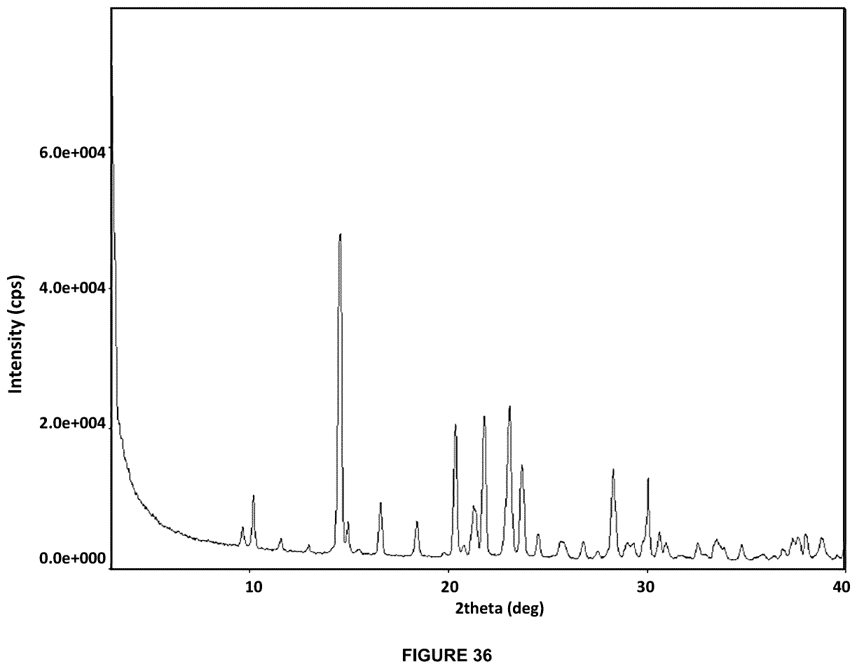

Solid 4-(3,3-difluoro-2,2-dimethylpropanoyl)-2,3,4,5-tetrahydropyrido[3,4-f][1,4]oxazepine-9-carbonitrile (˜100 mg) was added to a 2-dram vial and heated on a pie-block to >129° C. (melting point of Form A) resulting in a yellow oil. The vial was then flash cooled in a dry-ice/acetone bath to give a glassy yellow solid. DSC taken several hours later shows an exotherm at 87.8° C. followed by endotherm at peak 125.6° C. XRPD taken the following day appears mostly amorphous (see FIG. 35). This amorphous form was converted back to a crystalline form by heating at 90-100° C. on a pie-block (˜1-2 hr) and then allowing to cool to RT (see FIG. 36).

### Example 7: Organic Impurity Analysis

Organic Impurities. The three identified organic synthesis impurities are shown in Table 23, below. An area percentage of 0.10% is the identification threshold by HPLC, and the acceptable limit for any impurity is 0.20% based on area percentage by HPLC analysis. No impurity was observed in Compound 1 with the level above 0.10%.

TABLE 23
Structures of Organic Impurities

| | Chemical | | Source of |
|---|---|---|---|
| | Details | Structure | Impurity |
| Organic impurity #1 | Molecular formula: C₉H₉N₃O 2HCl Molecular Weight: 248.11 g/mol | 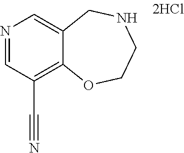 [SMILES: N#Cc1cncc2c1OCCNC2] | Final intermediate |
| Organic impurity #2 | Molecular formula: C₁₄H₁₇N₃O₃ Molecular Weight: 275.31 g/mol | 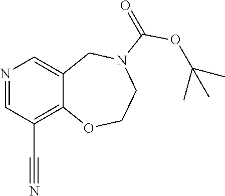 [SMILES: CC(C)(C)OC(=O)N1CCOc2c(C#N)cncc2C1] | Synthesis intermediate |
| Organic impurity #3 | Molecular formula: C₁₄H₁₉N₃O₄ Molecular Weight: 293.33 g/mol | 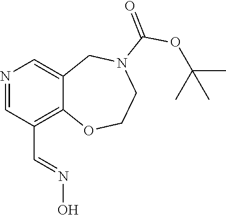 [SMILES: CC(C)(C)OC(=O)N1CCOc2c(/C=N/O)cncc2C1] | Synthesis intermediate |

The HPLC assay is as follows. The test is carried out by HPLC. Compound 1 content is calculated by external standardization on anhydrous and solvent free basis and from the average of two testing results.
Column: Waters Xbridge C18, 150 mm×4.6 mm ID., 3.5 μm, or equivalent
Mobile phase A: 0.05% (v/v) trifluoroacetic acid (TFA) in water
Mobile phase B: 0.05% (v/v) TFA in acetonitrile
Flow rate: 1.0 mL/min.
Injection volume: 5 μL
Sample temperature: 10° C.
Column temperature: 40° C.
Detection wavelength: 220 nm
Standard solution: 0.4 mg/mL of drug substance reference standard in acetonitrile/water (1:4, v/v). Test solution: 0.4 mg/mL of test sample in acetonitrile/water (1:4, v/v).
Gradient conditions: A gradient of mobile phase B and mobile phase A with the following composition:

| | Mobile | Mobile |
|---|---|---|
| Time | Phase B | Phase A |
| (min) | (% v/v) | (% v/v) |
| 0 | 5 | 95 |
| 1.00 | 5 | 95 |
| 16.00 | 50 | 95 |
| 20.00 | 100 | 0 |
| 21.00 | 100 | 0 |
| 21.50 | 5 | 95 |
| 30.00 | 5 | 95 |

Assay “as is” is the calculated Compound 1 content based on total impurities, total residual solvents, water content and residual on ignition in the drug substance.

Assay “as is” (% w/w)=(100%−total impurities %)×(100%-total residual solvents %−water content %−residual on ignition %)/100%.

Total impurities, total residual solvents and residual on ignition are measured by analytical procedures used to release Compound 1 and water content is measured according to USP<921>/Ph. Eur. <2.5.12>.

The same chromatographic conditions as for “assay (on anhydrous and solvent free bases) by HPLC” are used for impurities by HPLC. Impurities are calculated using the peak area percent method.

### Example 8: Preparation of Powder in Capsule Formulation Containing 5 mg, 10 mg, and 50 mg of Compound 1

Powder in Capsule (PiC) formulation was initially developed for Compound 1 as white opaque, size 1, hard gelatin capsules in three strengths, 5 mg, 10 mg, and 50 mg, for oral administration. The composition of Compound 1 5 mg, 10 mg, and 50 mg hard capsules is provided in Table 24.

TABLE 24
Composition of Compound 1 5 mg,
10 mg, and 50 mg hard capsules

| | Composition, mg/capsule | | |
|---|---|---|---|
| Components | 5 mg | 10 mg | 50 mg |
| Compound 1 | 5 mg | 10 mg | 50 mg |
| Hard gelatin | | 1 unit of | |
| capsule shell^{a} | | size “1” | |

^{a}Capsule shell is composed of gelatin (Ph. Eur.-NF) and coloring agent, 3.0% of titanium dioxide (E171/CI 77891, 231/2012/CE, Ph. Eur.-USP) as opacifier and white pigment function.

### Example 9: Preparation of Film Coated Tablets Containing Compound 1

A tablet formulation was then developed for subsequent studies. The excipients were selected taking into account the following requirements:
1. Compatibility with the drug substance and stability of the formulation
2. Functionality and technical performance in the manufacturing process
3. Development of multiple strengths (10 to 200 mg)
4. Drug substance/excipient ratio to ensure a good content uniformity
5. Dissolution rate
6. Resistance to crushing to withstand the administration by gastric tube
7. Compliance with regulations.

A binary compatibility study of the drug substance with excipients commonly used for tablet formulation was performed. In addition, four blend were manufactured by simple blending with typical tablet compositions at 10.0% loading of drug substance were studied, as summarized in Table 25. All samples were stored under 50° C./ambient (open/closed), 50° C./75% relative humidity (RH) (open/closed) and 25° C./60% RH (closed) conditions for 4 weeks. The samples were analysed for appearance, assay, and total related substances of Compound 1 after two and four weeks. While some of the binary mixtures of the drug substance with microcrystalline cellulose, croscarmellose sodium, and colloidal anhydrous silica showed incompatibility at accelerated conditions (i.e., 50° C., open conditions), all drug substance/excipient samples in binary mixtures were stable at 25° C./60% RH (closed). All four formulations were stable under all test conditions. Thus, it was concluded that all the excipients involved in the studies are suitable for the formulation development as they will be present at lower levels without exposure to high temperatures during manufacturing and storage.

TABLE 25
Tablet formulations for excipient compatibility study

| Formulation | Component | Function | % Weight | mg/bottle |
|---|---|---|---|---|
| Formulation | Compound 1 | Drug substance | 10.00 | 50 |
| A1 | Microcrystalline | Diluent | 75.00 | 375 |
| | cellulose | | | |
| | Hydroxypropyl | Binder | 5.00 | 25 |
| | cellulose | | | |
| | Croscarmellose | Disintegrant | 6.00 | 30 |
| | sodium | | | |
| | Colloidal | Glidant | 2.00 | 10 |
| | anhydrous silica | | | |
| | Magnesium | Lubricant | 2.00 | 10 |
| | stearate | | | |
| | Total | NA | 100.00 | 500.00 |
| Formulation | Compound 1 | Drug substance | 10.00 | 50 |
| A2 | Lactose | Diluent | 40.00 | 200 |
| | monohydrate | | | |
| | Mannitol | Diluent | 40.00 | 200 |
| | Sodium starch | Disintegrant | 6.00 | 30 |
| | Glycolate | | | |
| | Colloidal | Glidant | 2.00 | 10 |
| | anhydrous silica | | | |
| | Magnesium | Lubricant | 2.00 | 10 |
| | stearate | | | |
| | Total | NA | 100.00 | 500.00 |
| Formulation | Compound 1 | Drug substance | 10.00 | 50 |
| A3 | Microcrystalline | Diluent | 70.00 | 350 |
| | cellulose | | | |
| | Hydroxypropyl | Binder | 5.00 | 25 |
| | cellulose | | | |
| | Croscarmellose | Disintegrant | 6.00 | 30 |
| | sodium | | | |
| | Sodium lauryl | Surfactant | 5.00 | 25 |
| | sulfate | | | |
| | Colloidal | Glidant | 2.00 | 10 |
| | anhydrous silica | | | |
| | Magnesium | Lubricant | 2.00 | 10 |
| | stearate | | | |
| | Total | NA | 100.00 | 500.00 |
| Formulation | Compound 1 | Drug substance | 10.00 | 50 |
| A4 | Lactose | Diluent | 37.50 | 187.5 |
| | monohydrate | | | |
| | Mannitol | Diluent | 37.50 | 187.5 |
| | Sodium starch | Disintegrant | 6.00 | 30 |
| | Glycolate | | | |
| | Sodium lauryl | Surfactant | 5.00 | 25 |
| | sulfate | | | |
| | Colloidal | Glidant | 2.00 | 10 |
| | anhydrous silica | | | |
| | Magnesium | Lubricant | 2.00 | 10 |
| | stearate | | | |
| | Total | NA | 100.00 | 500.00 |

Tablet batches ranging from 10 to 200 mg (drug load from 10 to 40%, drug load refers to the percentage of Compound 1 in the formulation) of dosage strengths were prepared to evaluate wet and dry granulation processes, and to compare different diluents (microcrystalline cellulose, mannitol and lactose), different disintegrants (croscarmellose sodium and sodium starch glycolate), different levels of binder (hydroxypropyl cellulose), and the lubricant (magnesium stearate). The tablet batches were tested in dissolution and stability studies. As some batches from the wet granulation process exhibited significant decrease in dissolution rate after one month of storage under 40° C./75% RH condition, the dry granulation process was therefore selected. Based on their functionality and compatibility with the drug substance, mannitol, lactose monohydrate, sodium starch glycolate, colloidal anhydrous silica and magnesium stearate were selected for the core tablet formulation.

The composition of the Formulation B1-B4 of Compound 1 film-coated tablets is provided in Table 26. The same centesimal composition was used for all tablet strengths. Therefore, the Formulation B1 and Formulation B2 were used for the validation of the HPLC analytical procedures and for supportive, bracketing stability studies.

The film-coating was developed to facilitate handling of tablets during production (e.g. packaging), to improve the tablet appearance and to aid the patient's ability to swallow. The film-coating composition selected comprised a fixed combination of hypromellose, titanium dioxide, and macrogol (polyethylene glycol), suitable for an immediate-release type dosage form.

TABLE 26
Composition of Formulation B1-B4 of Compound 1 film-coated tablets

| | Composition, mg/tablet (% w/w) | | | |
|---|---|---|---|---|
| | 10 mgª | 50 mg | 10 mg | 20 mg |
| | (Formulation | (Formulation | (Formulation | (Formulation |
| Components | B1 | B2 | B3) | B4) |
| Intragranular | | | | |
| Compound 1 | 10.00 (10.00) | 50.00 (10.00) | 10.00 (10.00) | 20.00 (10.00) |
| Mannitol | 25.50 (25.50) | 127.50 (25.50) | 25.50 (25.50) | 51.00 (25.50) |
| Lactose | 59.25 (59.25) | 296.25 (59.25) | 59.00 (59.00) | 118.00 (59.00) |
| monohydrate | | | | |
| Sodium starch | 2.00 (2.00) | 10.00 (2.00) | 2.00 (2.00) | 4.00 (2.00) |
| glycolate | | | | |
| Colloidal | 0.25 (0.25) | 1.25 (0.25) | 0.25 (0.25) | 0.50 (0.25) |
| anhydrous | | | | |
| silica | | | | |
| Magnesium | 0.25 (0.25) | 1.25 (0.25) | 0.25 (0.25) | 0.50 (0.25) |
| stearate | | | | |
| Extragranular | | | | |
| Sodium starch | 2.00 (2.00) | 10.00 (2.00) | 2.00 (2.00) | 4.00 (2.00) |
| glycolate | | | | |
| Magnesium | 0.75 (0.75) | 5.00 (1.00) | 1.00 (1.00) | 2.00 (1.00) |
| stearate | | | | |
| Core tablet | 100.00 | 501.25 | 100.00 | 200.00 |
| mass | (100.00) | (100.25) | (100.00) | (100.00) |
| Film-coating components | | | | |
| Aquarius ™ BP | 3.00 (3.00) | 15.04 (3.00) | 3.00 (3.00) | 6.00 (3.00) |
| 18237 white | | | | |
| film-coating | | | | |
| system | | | | |
| Film-coated | 103.00 | 516.29 | 103.00 | 206.00 |
| tablet total | (103.00) | (103.226) | (103.00) | (103.00) |

^{a}A slight difference (0.25% w/w) of magnesium stearate extragranular quantities for the Formulation B1 of 10 mg film-coated tablets in comparison to other film-coated tablets was compensated by the lactose monohydrate (diluent excipient) quantities.
^{b}Aquarius ™ BP 18237 white is composed of 63.0% hypromellose 6 mPa · s (Ph. Eur.-USP-JP), 30% titanium dioxide (E171) (Ph. Eur.-USP-JP) and 7% macrogol (polyethylene glycol) (Ph. Eur.-NF-JP).

### Example 10: Process for Preparing Film-Coated Tablets

The process for preparing film-coated tablets includes the steps of roller compaction granulation (dry granulation), compression, followed by film-coating.

Step 1: All components were weighed according to the tablet formulations of Table 27.

Step 2: Compound 1 was sifted with part of lactose monohydrate, then mannitol, sodium starch glycolate, and remaining part of lactose monohydrate through a screen mesh size comprised between 0.42 to 2.00 mm.

Step 3: Sieved components were mixed in a tumble blender.

Step 4: Colloidal anhydrous silica and magnesium stearate were sifted through a screen mesh size comprised between 0.25 to 0.50 mm.

Step 5: The sieved anhydrous silica and magnesium stearate was loaded into the same tumble blender and mixed for lubrication for about 5 to 10 minutes.

Step 6: The intragranular blend was passed through a roller compactor equipped with an integrated milling, including a first coarse screen and a second mill screen of 1.00 mm. The dry roller compaction process developed was selected as an appropriate granulation method for low dose product to ensure a good content uniformity and stability of the formulation. Standard equipment typical of the pharmaceutical process was used for the manufacture of Compound 1 tablets.

Step 7: Sodium starch glycolate was sifted through a screen mesh size comprised between 0.42 to 2.00 mm.

Step 8: Sieved sodium starch glycolate was mixed with the granules produced from step 6.

Step 9: Magnesium stearate was sifted through a screen mesh size comprised between 0.25 to 0.50 mm.

Step 10: Sieved magnesium stearate was added to the blend obtained at step 8 and was proceeded to final mixing for about 35 rotations to obtain a homogeneous blend.

Step 11: The lubricated granules were compressed on a tablet press fitted with neutral round-shape punches at a nominal mass of 200 mg per tablet.

Step 12: In a mixing tank, a sufficient quantity of film coating suspension with Aquarius™ BP 18237 white and purified water was prepared.

Step 13: The tablets obtained at step 11 were placed in a film-coating pan and sprayed with the film-coating suspension onto the cores maintaining an outlet air temperature between 40° C. to 45° C. The film-coating process was continued until 6 mg of film-coating material was applied per tablet. Table 27 shows the composition of the tablets prepared using this method.

TABLE 27
Composition of 20 mg film-coated tablets containing Compound 1
(Formulation B4)

| | Composition | | | |
|---|---|---|---|---|
| | (Formulation B4) | | | Reference to |
| | % | mg/ | | |
| Components | weight | tablet | Function | standardsª |
| Intragranular | | | | |
| Compound 1 | 10.00 | 20.00 | Drug | |
| | | | substance | |
| Mannitol | 25.50 | 51.00 | Diluent | Ph. Eur.-USP-JP |
| Lactose | 59.00 | 118.00 | Diluent | Ph. Eur.-NF-JP |
| monohydrate | | | | |
| Sodium starch | 2.00 | 4.00 | Disintegrant | Ph. Eur.-NF-JP |
| glycolate | | | | |
| Colloidal | 0.25 | 0.50 | Glidant | Ph. Eur.-NF |
| anhydrous silica- | | | | |
| Colloidal silicon | | | | |
| dioxide | | | | |
| Magnesium | 0.25 | 0.50 | Lubricant | Ph. Eur.-NF-JP |
| stearate^{b} | | | | |
| Extragranular | | | | |
| Sodium starch | 2.00 | 4.00 | Disintegrant | Ph. Eur.-NF-JP |
| glycolate | | | | |
| Magnesium | 1.00 | 2.00 | Lubricant | Ph. Eur.-NF-JP |
| stearate^{b} | | | | |
| Core tablet mass | 100.00 | 200.00 | | |
| Film-coating | | | | |
| components | | | | |
| Aquarius ™ BP | 3.00 | 6.00 | Film- | |
| 18237 white | | | coating | |
| film-coating | | | components | |
| agent | | | | |
| Purified water^{c} | | | Processing | Ph. Eur.-USP-JP |
| | | | agent | |
| Film-coated | 103.00 | 206.00 | | |
| tablet mass | | | | |

^{a}Reference is made to the current edition of the Pharmacopoeia.
^{b}From vegetable origin.
^{c}Water is used as the film-coating solvent and removed during manufacture.

Cohesion is one of the critical material attributes (CMAs) which impact the flowability of pharmaceutical powders and hence the successful formulation of solid dosage forms. Evaluation of the physical characteristics of Compound 1 shows that Compound 1 has a very low cohesion property. Different options were explored to improve the cohesion property of the tablet, for example, by change of lactose and mannitol grades (standard grade in place of Spray Dried grade), using anhydrous lactose in place of monohydrate lactose Spray-Dried, using different ratios of lactose/mannitol, or using pregelatinized corn starch instead of mannitol, but only a decrease of tablet cohesion was observed. For formulation B5, a mixture of anhydrous lactose and microcrystalline cellulose were selected and the ratio of anhydrous lactose/cellulose in formulation B5 selected resulted in improved cohesion property.

Table 28 shows a composition of a film-coated tablet prepared by dry granulation (compaction).

TABLE 28
Composition of Compound 1 20 mg
film-coated tablet (Formulation B5)

| | Unit quantity | | |
|---|---|---|---|
| | (200 mg/ | Percentage | |
| Components | core tablet) | (w/w) | Function |
| Intragranular | | | |
| Compound 1 | 20.00 | 10.00 | Drug |
| | | | substance |
| Anhydrous lactose | 109.50 | 54.75 | Diluent |
| Microcrystalline cellulose | 58.00 | 29.00 | Diluent |
| PH102 | | | |
| Sodium starch glycolate | 8.00 | 4.00 | Disintegrant |
| Colloidal silicon dioxide | 0.50 | 0.25 | Glidant |
| Magnesium stearate | 0.50 | 0.25 | Lubricant |
| Extragranular | | | |
| Sodium starch glycolate | 2.00 | 1.00 | Disintegrant |
| Magnesium stearate | 1.50 | 0.75 | Lubricant |
| Core tablet mass | 200.00 | 100.00 | |
| Film-coating | | | |
| Aquarius ™ BAP310227 | 6.00 | 3.00 | Film-coating |
| Pink-Ashland^{a} | | | agent |
| Purified water^{b} | q.s | q.s. | Film-coating |
| | | | solvent |
| Film-coated tablet mass | 206.00 | 103.00 | |

^{a}Aquarius ™ BAP310227 Pink is composed of 62.50% hypromellose 29106 mPa · s, 24.06% titanium dioxyde, 12.50.% polyethylene glycol, 0.52% iron oxide red and 0.42% iron oxide yellow.
^{b}Removed during process.

Example 11.1: Formulation Development of Oral Suspension for G-Tube Administration

Due to the specific diseases or conditions mediated by RIPK1, some patients could have swallowing difficulties. In such a case, an oral suspension may be prepared extemporaneously and delivered by a Gastrostomy-tube (G-tube). The content of the film-coated tablets (20 mg film-coating tablet or the placebo for Compound 1 20 mg film-coated tablets) can be dispersed in tap water at room temperature as usually performed with such a medical device in order to reconstitute the suspension at the required dose.

The operating conditions recommended for the preparation of an oral suspension for further analytical and/or clinical studies are as follows.
Use a 60 mL syringe for enteral feeding with ENFit connector and a glass vial for the suspension preparation
Sample a large volume of tap water at room temperature in a second large glass vial (˜ 150 mL)
Keep the preparation at ambient temperature until administration.
Before suspension administration, draw 10 mL of tap water at room temperature into the syringe to first rinse the gastric tube.

To support the administration by a G-tube, an in-use study was performed with a reconstituted suspension, prepared from Compound 1 20 mg film-coated tablets. The in-use study covered the storage time (15 minutes) between the extemporaneous preparation and administration of the suspension. The aim of the in-use study was to ensure that the complete dose of Compound 1 20 mg film-coated tablets can be delivered to the patient by G-tube administration.

### Protocol for Preparation of an Oral Suspension

1. 40 mL of tap water was drawn into the 60 mL syringe from the 150 mL glass vial and poured into the empty glass vial.
2. The tablet was placed in the water.
3. The tablet was let to sit in the water for 10 minutes to allow swelling and dispersion.
4. After this 10-minute period, the remaining tablet pieces were crushed with the back of a little spoon.
5. The vial contents were gently mixed to obtain a visually homogeneous suspension.
6. The entire suspension was drawn into the 60 ml syringe
7. The opening of the syringe was connected to the gastric tube and the contents of the syringe were emptied into the gastric tube.
8. The syringe was disconnected from the gastric tube. Another 40 mL of tap water was drawn into the syringe and emptied it into the glass vial to rinse all items (vial, spoon and syringe).
9. The totality of the rinse water was drawn back into the syringe.
10. The syringe was connected to the gastric tube and its contents were emptied into the gastric tube (1st rinsing).
11. The syringe was disconnected from the gastric tube for a second rinsing. 20 mL of tap water was drawn into the syringe and emptied into the glass vial to rinse all items (vial, spoon and syringe).
12. The totality of the rinse water was then drawn into the syringe.
13. The syringe was connected to the gastric tube and emptied into the gastric tube (2nd rinsing).
14. The administration was done within 15 minutes of preparing the suspension.

Example 11.2: In-use study with an extemporaneous, oral suspension of 20 mg of Compound 1 film-coated tablets (Formulation B4 or Formulation B5) in water. The oral suspension used in this study was prepared as described in Example 11.1.

To support the administration by a G-tube, an in-use study was performed with a reconstituted oral suspension, prepared from Compound 1 20 mg film-coated tablets (Formulation B4 or B5) in water. The in-use study combined a recovery and a stability study over 30 minutes at room temperature, to cover the potential storage time (15 minutes) after the suspension preparation.

For the syringes, sterile, commercially available syringes for enteral feeding with a volume of up to 60 mL were used. Two G-tube materials were tested: polyurethane or silicone.

The same operating conditions were used for the recovery and the stability study, based on the preparation protocol. The assay by UHPLC was determined each time in two steps after the G-tube administration: one after the first rinsing and one after the second rinsing step.

The final dose recoveries were calculated between the sum of these two assay determinations and the initial assay result of 20 mg film-coated tablets.

In addition, for the in-use stability study, the impurity profiles by UHPLC were assessed after a storage time of 30 min.

The appearance was verified for the recovery and the stability study.

After 30 min storage at room temperature, the appearance of Compound 1 suspension in water remained unchanged, the related substances remained <0.10%, and no significant change of the pH was observed. All the recoveries obtained for the assay were between 99% and 102% after the simulation of the G-tube administration with or without a 30-min delay after administration and met the acceptance criteria (between 90% and 110%).

Based on the above data, the Compound 1 suspension in water could be stored at room temperature up to 30 min prior to administration and was compatible with the G-tube materials tested.

### Example 12: Analytical Tests

The following Table 29 lists tests, analytical procedures, and preliminary acceptance criteria for Compound 1 20 mg film-coated tablets. Pharmacopoeial analytical procedure and acceptance criteria references refer to the current versions of the pharmacopoeias including their supplements.

TABLE 29
Specifications for Compound 1 20 mg film-coated tablets

| | Analytical | |
|---|---|---|
| Test | procedure | Acceptance criteria |
| Appearance | Visual | White to off-white film |
| | | coated round tablet |
| Identification | UHPLC | Corresponds to retention |
| (retention time) | | time of reference ±5% |
| Identification (UV) | UHPLC/UV or | Conforms to reference |
| | UV^{a} | |
| Assay (%) | UHPLC | 90.0-110.0 label claim |
| | | (18.0 to 22.0 mg of |
| | | Compound 1 per tablet) |
| Degradation | UHPLC | |
| products/Impurities^{b} | | |
| (area %) | | |
| Any | | ≤0.20 |
| unspecified | | |
| degradation | | |
| product/ | | |
| impurity | | |
| Total | | ≤2.0 |
| degradation | | |
| products/ | | |
| impurities | | |
| Uniformity of | UHPLC | Complies with USP<905>/ |
| dosage units/ | | Ph. Eur.<2.9.40>/JP6.02 |
| Content uniformity | | (harmonized) |
| Disintegration | USP<701>, Ph. | ≤0 |
| (minutes) | Eur.<2.9.1>/ | |
| | JP6.09 | |
| | (ICH harmonized) | |
| Dissolution (30 min, | Assay by UV | |
| %) | spectrophotometry | |
| Min, Max, Mean | | To be determined^{c} |
| and RSD | | |
| Microbial | Ph. Eur. 2.6.12/ | ≤10³ |
| examination test | USP<61>/JP4.05 | ≤10² |
| | Part 1 | |
| TAMC^{d} | | |
| (CFU/g) | | |
| TYMC^{e} | | |
| (CFU/g) | | |
| Specified | Ph. Eur. 2.6.13/ | |
| microorganisms | USP<62>/JP4.05 | Absence in 1 g |
| Escherichia | Part 2 | |
| coli | | |

^{a}Or UV spectrum recorded on standard UV spectrophotometer
^{b}Includes drug substance synthesis impurities at this stage of development.
^{c}Dissolution profile is also monitored for development database purposes.
^{d}TAMC: Total Aerobic microbial count
^{e}TYMC: Total Yeasts and Molds count

### Analytic Procedure by HPLC

Column: Waters Xbridge C18, 150 mm×4.6 mm ID., 3.5 μm, or equivalent
Mobile phase A: 0.05% (v/v) trifluoroacetic acid (TFA) in water
Mobile phase B: 0.05% (v/v) TFA in acetonitrile
Flow rate: 1.0 mL/min.
Injection volume: 5 μL
Sample temperature: ambient temperature
Column temperature: 40° C.
Detection wavelength: 220 nm
Standard solution: 0.5 mg/mL of drug substance reference standard in acetonitrile/water (4:1, v/v)
Test solution: 0.5 mg/mL of test sample in acetonitrile/water (4:1, v/v)
10 mg film-coated tablet: 5 tablets dissolved in 100 mL acetonitrile/water (4:1, v/v)
20 mg film-coated tablet: 5 tablets dissolved in 200 mL acetonitrile/water (4:1, v/v)
50 mg film-coated tablet 5 tablets dissolved in 500 mL acetonitrile/water (4:1, v/v)
Gradient conditions: A gradient of mobile phase B and mobile phase A with the following composition:

| | Mobile | Mobile |
|---|---|---|
| Time | Phase B | Phase A |
| (min) | (% v/v) | (% v/v) |
| 0 | 5 | 95 |
| 1.00 | 5 | 95 |
| 16.00 | 50 | 50 |
| 20.00 | 100 | 0 |
| 21.00 | 100 | 0 |
| 21.50 | 5 | 95 |
| 30.00 | 5 | 95 |

### Analytic Procedure by UHPLC

Column: Acquity CSH C18, 100 mm×2.1 mm ID., 1.7 μm, (Waters manufacturer) or equivalent
Mobile phase A: 0.005M ammonium acetate in water
Mobile phase B: Acetonitrile
Flow rate: 0.4 mL/min.
Injection volume: 3 μL
Sample temperature: 10° C.
Column temperature: 40° C.
Detection wavelength: 230 nm
Standard solution: 0.2 mg/mL of drug substance reference standard in acetonitrile/water (20/80 v/v)
Test solution: 0.2 mg/mL of test sample in acetonitrile/water (20/80 v/v)
Gradient conditions: A gradient of mobile phase B and mobile phase A with the following composition:

| | Mobile | Mobile |
|---|---|---|
| Time | Phase B | Phase A |
| (min) | (% v/v) | (% v/v) |
| 0 | 5 | 95 |
| 0.4 | 5 | 95 |
| 4.0 | 30 | 70 |
| 6.0 | 30 | 70 |
| 8.0 | 50 | 50 |
| 9.0 | 95 | 5 |
| 10.0 | 95 | 5 |
| 10.5 | 5 | 95 |
| 13.0 | 5 | 95 |

A photostability assessment was conducted on Compound 1 20 mg film-coated tablets, under ICH Q1B (Option 2) conditions.

The photostability of Compound 1 20 mg film-coated tablets was evaluated per ICH Q1B option 2 conditions. The samples were placed in open petri dishes and exposed to visible and UV-A illumination under the following conditions: overall illumination of NLT 1.2 million lux hours and an integrated near UV energy of NLT 200 watt hours/square meter.

The investigations on the physical and chemical properties of 20 mg Compound 1 film-coated tablets stored under photostability storage conditions showed no significant change of the specified test items and therefore confirmed no photosensitivity (see Table 30).

TABLE 30
Drug product photostability for Compound 1 20 mg film-coated tablets
(Formulation B5)

| | | Condition | |
|---|---|---|---|
| | Acceptance | | ICH Q1B |
| Test item | criteria | Initial results | Option 2 |
| Appearance | White to | White, round | White, round |
| | off-white | tablet | tablet |
| | round tablet | | |
| Assay/(%) | 90.0-110 label | 100.3 | 101.1 |
| Degradation | claim | | |
| products/ | | | |
| Impurities^{a}/ | | | |
| (area %) | | | |
| Total | NMT 2.0 | 0.06 | 0.06 |
| degradation | | | |
| products/ | | | |
| Impurities | | | |
| Any | NMT 0.20 | 0.06 (Rrt = | 0.06 (Rrt = |
| unspecified | | 1.11) | 1.10) |
| degradation | | | |
| product | | | |
| Disintegration | ≤30 | <3 | <3 |
| (minutes) | | | |
| Water content | Report | 3.6 | 3.7 |
| (% w/w) | | | |
| XRPD | Crystalline | Crystalline | Crystalline |
| | (Consistent with | (Consistent with | (Consistent with |
| | Form A) | Form A) | Form A) |

^{a}Includes drug substance synthesis impurities at this stage of development.

XRPD analysis is carried out according to US Pharmacopeia <941>. According to the stability testing, XRPD analysis of tablets from the photostability test shows that Compound 1 has a crystalline form consistent with Form A after 6 months of storage.

The drug product stability for the 10 mg and 50 mg Compound 1 film-coated tablets (Formulation B1 and B2, respectively) in blisters with PVC-PCTFE forming film and hardened aluminum foil lidding stored under +30±2° C./65%±5%, +40±2° C./75%±5% RH, +5±3° C. were evaluated. The physical and chemical properties (in terms of appearance, assay, degradation products/impurities, disintegration, dissolution, water content, and XRPD) confirmed good stability after one month, three months and six months of storage with no significant trend. The XRPD confirmed Compound 1 in the film-coated tablets is crystalline, with a form consistent with Form A after six months of storage.

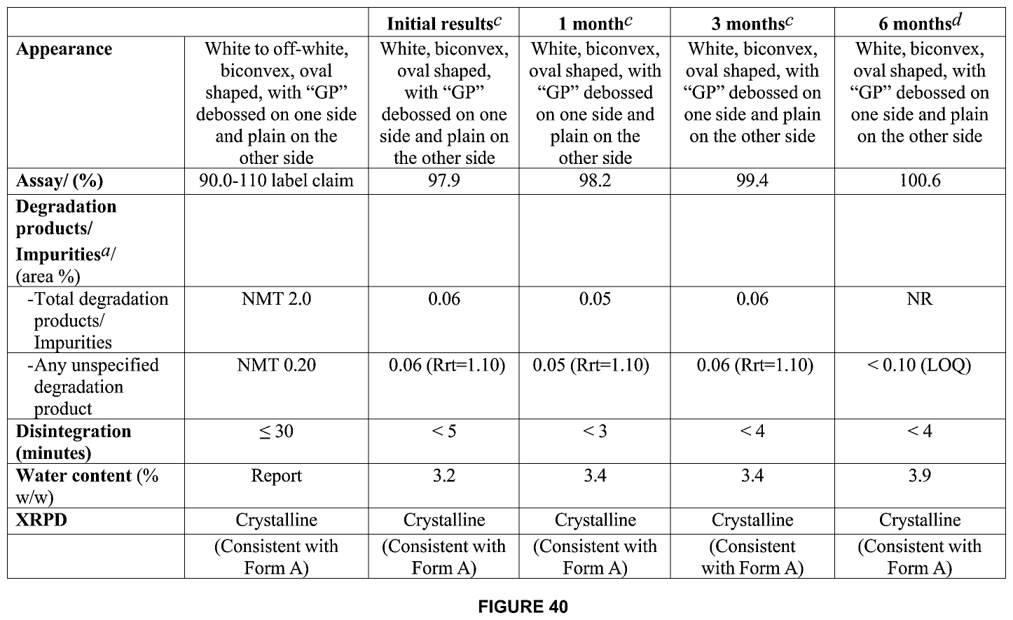

FIG. 40 is a table that shows drug product stability data for 10 mg tablet (Formulation B1) in blisters +40±2° C./75%±5% RH: a Includes drug substance synthesis impurities, c Analytic procedure by HPLC in Example 12, and d Analytic procedure by UHPLC in Example 12.

The above-mentioned supportive stability results for Compound 1 10 and 50 mg film-coated tablets (Formulation B1 and B2, respectively) showed that the drug product (homothetic formulation) remains stable for 6 months when stored at long term storage conditions (30° C./65% RH) and at accelerated storage conditions (+40±2° C./75±5% RH) in Al/PVC-PCTFE blisters. Based upon the above-mentioned investigations, as well as the available photostability results for Compound 1 20 mg film-coated tablets (Formulation B5), the following preliminary storage recommendation was set:
Preliminary shelf life: 18 months
Preliminary storage direction: Store between 2° C. and 30° C.

In addition, an in-use study was performed with a reconstituted oral suspension, prepared from Compound 1 20 mg film-coated tablets in water, to support the administration of an extemporaneous oral suspension by G-tube (polyurethane or silicone).

After 30 min storage at room temperature the appearance of Compound 1 suspension in water remains unchanged, the related substances remain <0.10% and no significant change of the pH is observed. All the recoveries obtained for the assay are between 99% and 102% after the simulation of the G-tube administration with or without a 30-min delay after administration and meet the acceptance criteria (between 90% and 110%).

Based on the above data, the Compound 1 suspension in water can be stored at room temperature up to 30 min prior to administration and is compatible with the G-tube materials tested.

### Example 13: Dissolution of 20 mg Tablets

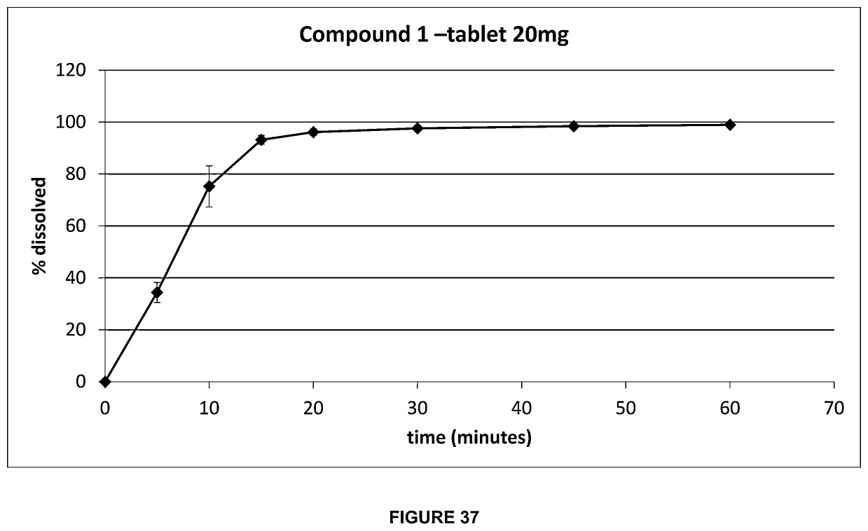

The dissolution of the tablet (Formulation B5) was tested according to the analytical methods described in Example 12. FIG. 37 shows that a plateau was reached in 20 minutes and about 93% dissolution was reached in 15 minutes.

### Example 14: Comparative Hardness Study

Ejection force is the force required to eject a tablet and is a critical measure in the tableting process. An excessive value can damage the tablet, reduce tool life, wear the equipment or stop the machine.

Tablet hardness can be used as a quality control indicator. Tablets should not be too hard or too soft. Extremely hard tablets may indicate that the binding force between the active ingredient and the excipient is too great, which may prevent the proper dissolution of the tablet needed for an accurate dosage. Hard tablets may prevent tablets from an appropriate disintegration during administration by gastric tube. Similarly, softer tablets may be the result of weak binding and may cause process issues during tablet film coating or packaging. Poor hardness tablets may also lead to tablet breakage during deblisterisation and impact patient compliance or the dose administered. Tablet hardness can be measured using a hardness tester.

In addition, patient centricity was taken into consideration and excipients and ratios were selected to ensure tablet crushability and dispersibility for quantitative administration as an extemporaneous suspension via a gastric tube.

For the Compound 1, a tablet with an extremely high hardness may limit the ability of the tablet to be administered by gastric tube: indeed, as per recommended by the physicians, the tablet should be soft enough to facilitate tablet dispersion in tap water at room temperature in a limited time.

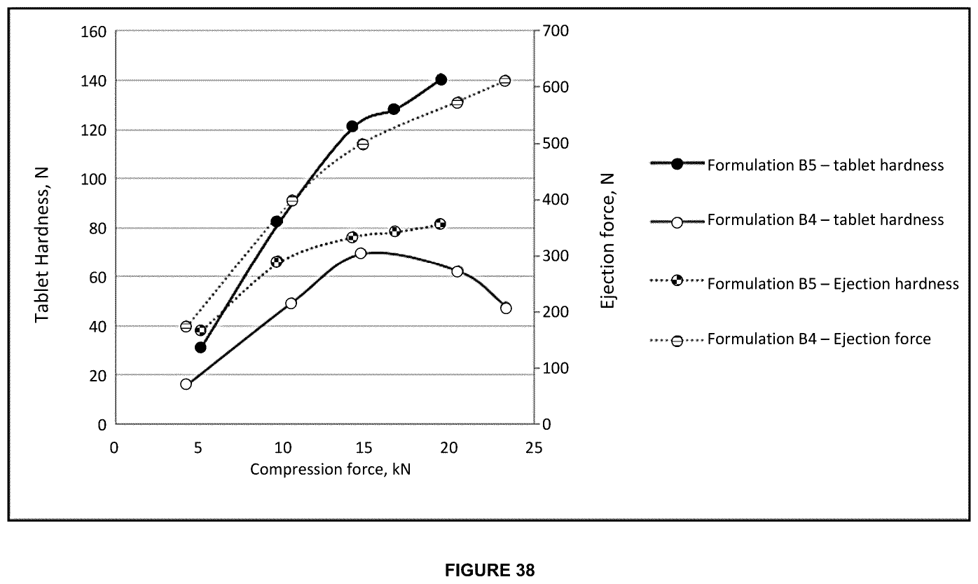

Tablets can be compared regarding tablet hardness and ejection force. Improvement of linearity of tablet hardness and ejection force versus compression force was observed for the tablet Formulation B5 in comparison to Formulation B4 (FIG. 38).

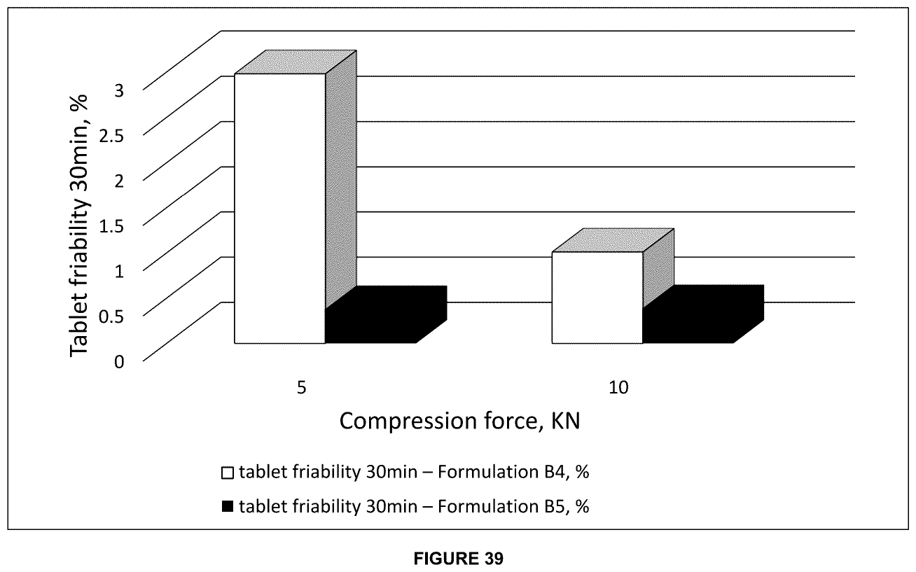

Tablets can also be compared regarding tablet friability, which was measured using a friability tester after 4 min and 30 min. Tablets were manufactured at the machine speed of 40 tr/min and a compression force of 5 and 10 kN for the tablet described in Formulation B4 in comparison to Formulation B5 is shown in Table 31 and FIG. 39.

TABLE 31
Tablet friability of Formulation B4 and B5

| | Compression | Compression |
|---|---|---|
| | Force 5 kN | Force 10 kN |
| Friability | 2.98 | 1.01 |
| (Formulation B5) | | |
| Friability | 0.38 | 0.39 |
| (Formulation B4) | | |

The present disclosure includes, for example, any one or a combination of the following embodiments:

Embodiment 1: A tablet comprising 4-(3,3-difluoro-2,2-dimethyl-propanoyl)-3,5-dihydro-2H-pyrido[3,4-f][1,4]oxazepine-9-carbonitrile (Compound 1) and at least one pharmaceutically acceptable excipient.

Embodiment 2: The tablet of embodiment 1, further comprising at least one coating layer.

Embodiment 3: The tablet of embodiments 1 or 2, wherein the at least one coating layer comprises hydroxypropyl methylcellulose.

Embodiment 4: The tablet of any one of the preceding embodiments, wherein the at least one coating layer comprises at least one of titanium dioxide and polyethylene glycol.

Embodiment 5: The tablet of any one of the preceding embodiments, wherein the at least one pharmaceutically acceptable excipient comprises at least one diluent.

Embodiment 6: The tablet of embodiment 5, wherein the at least one diluent is chosen from mannitol, lactose monohydrate, anhydrous lactose, microcrystalline cellulose, starch, sorbitol, dextrose, dibasic calcium phosphate, dicalcium phosphate dihydrate, tricalcium phosphate, calcium phosphate, pregelatinized starch, compressible sugar, hydroxypropyl-methylcellulose, hydroxypropylmethylcellulose acetate stearate, sucrose-based diluents, confectioner's sugar, monobasic calcium sulfate monohydrate, calcium sulfate dihydrate, calcium lactate trihydrate, a dextrate, hydrolyzed cereal solids, amylose, powdered cellulose, calcium carbonate, glycine, kaolin, sodium chloride, inositol, and bentonite.

Embodiment 7: The tablet of embodiments 5 or 6, wherein the at least one diluent is present in a total amount of diluent ranging from about 45 to about 90% by weight of the tablet.

Embodiment 8: The tablet of any one of embodiments 5-7, wherein the at least one diluent is present in a total amount of diluent ranging from about 75 to about 90% by weight of the tablet.

Embodiment 9: The tablet of any one of the preceding embodiments, wherein the at least one pharmaceutically acceptable excipient comprises at least one glidant.

Embodiment 10: The tablet of embodiment 9, wherein the at least one glidant is chosen from colloidal anhydrous silica, and colloidal silicon dioxide.

Embodiment 11: The tablet of embodiments 9 or 10, wherein the at least one glidant is present in a total amount of glidant ranging from about 0.1 to about 5% by weight of the tablet.

Embodiment 12: The tablet of any one of the preceding embodiments, wherein the at least one pharmaceutically acceptable excipient comprises at least one disintegrant.

Embodiment 13: The tablet of embodiment 12, wherein the at least one disintegrant is chosen from sodium starch glycolate, croscarmellose sodium, corn starch, potato starch, pregelatinized starch, methylcrystalline cellulose, methylcellulose, croscarmellose, cross-linked sodium carboxymethyl-cellulose, cross-linked carboxymethylcellulose, cross-linked croscarmellose, crosspovidone, cross-linked polyvinylpyrrolidone, alginic acid, sodium alginate, magnesium aluminum silicate, agar, guar, locust bean, Karaya, pectin, tragacanth, bentonite, citrus pulp, and sodium lauryl sulfate.

Embodiment 14: The tablet of embodiments 12 or 13, wherein the at least one disintegrant is present in a total amount of disintegrant ranging from about 1 to about 15% by weight of the tablet.

Embodiment 15: The tablet of any one of the preceding embodiments, wherein the at least one pharmaceutically acceptable excipient comprises at least one lubricant.

Embodiment 16: The tablet of embodiment 15, wherein the at least one lubricant is chosen from magnesium stearate, stearic acid, calcium hydroxide, talc, sodium stearyl fumerate, mineral oil, hydrogenated soybean oil, aluminum, calcium, magnesium, zinc, sodium stearate, glycerol, talc, wax, boric acid, sodium benzoate, sodium acetate, sodium chloride, leucine, polyethylene glycol, methoxypolyethylene glycol, sodium oleate, sodium benzoate, glyceryl behenate, magnesium lauryl sulfate, sodium lauryl sulfate, colloidal silica, corn starch, silicone oil, and surfactant.

Embodiment 17: The tablet of embodiments 15 or 16, wherein the at least one lubricant is present in a total amount of lubricant ranging from about 0.1 to about 5% by weight of the tablet.

Embodiment 18: The tablet of any one of the preceding embodiments, wherein the tablet comprises an intragranular core and an extragranular portion.

Embodiment 19: The tablet of embodiment 18, wherein the intragranular core comprises at least one diluent, at least one disintegrant, at least one glidant, and at least one lubricant.

Embodiment 20: The tablet of embodiments 18 or 19, wherein the extragranular portion comprises at least one disintegrant and at least one lubricant.

Embodiment 21: The tablet of any one of the preceding embodiments, wherein Compound 1 is present in at least 50% crystalline form, such as at least 60%, at least 70%, at least 80%, at least 90%, at least 95%, at least 96%, at least 97%, at least 98%, at least 99%, 99.5%, or 100% crystalline.

Embodiment 22: The tablet of embodiment 21, wherein the crystalline form is Form A.

Embodiment 23: The tablet of any one of the preceding embodiments, wherein Compound 1 is present in an amorphous form.

Embodiment 24. The tablet of any one of claims 1 to 21, wherein Compound 1 is present in crystalline form A.

Embodiment 25: The tablet of any one of the preceding embodiments, wherein Compound 1 is present in an amount ranging from about 5 mg to about 60 mg.

Embodiment 26: The tablet of any one of the preceding embodiments, wherein Compound 1 is present in an amount of about 5 mg, about 10 mg, about 15 mg, about 20 mg, about 25 mg, about 30 mg, about 35 mg, about 40 mg, about 45 mg, about 50 mg, about 55 mg, or about 60 mg.

Embodiment 27: The tablet of any one of the preceding embodiments, wherein Compound 1 is present in an amount of about 20 mg.

Embodiment 28: The tablet of any one of the preceding embodiments, wherein the tablet comprises 45 to 65% by weight anhydrous lactose, 20 to 40% by weight microcrystalline cellulose, 1 to 10% by weight sodium starch glycolate, 0.1 to 5% by weight colloidal silicon dioxide, and 0.1 to 5% by weight magnesium stearate.

Embodiment 29: The tablet of any one of embodiments 1-27, wherein the tablet comprises 20 to 30% by weight mannitol, 50 to 70% by weight lactose monohydrate, 0.5 to 8% by weight sodium starch glycolate, 0.1 to 1% by weight colloidal anhydrous silica, 0.1 to 1% by weight colloidal silicon dioxide, and 0.1 to 5% by weight magnesium stearate.

Embodiment 30: The tablet of any one of the preceding embodiments, further comprising at least one of a pH adjusting agent, a salt, an antifoaming agent, an antioxidant, a preservative, a dispersing agent, a flavoring agent, a solubilizer, a plasticizer, a suspending agent, a surfactant, a viscosity enhancing agent, and a wetting agent.

Embodiment 31: The tablet of any one of the preceding embodiments, wherein the tablet has a hardness of 60 N to 110 N.

Embodiment 32: The tablet of any one of the preceding embodiments, wherein the tablet has a hardness of 85 N.

Embodiment 33: A pharmaceutical formulation comprising Compound 1 and at least one pharmaceutically acceptable excipient, wherein the at least one pharmaceutically acceptable excipient comprises:
at least one diluent selected from the group consisting of lactose, sucrose, dextrose, dextrates, maltodextrin, mannitol, xylitol, sorbitol, cyclodextrins, calcium phosphate, calcium sulfate, starches, modified starches, cellulose, microcrystalline cellulose, microcellulose, and talc;
at least one disintegrating agent selected from the group consisting of natural starch, a pregelatinized starch, sodium starch, methylcrystalline cellulose, methylcellulose, croscarmellose, croscarmellose sodium, cross-linked sodium carboxymethylcellulose, cross-linked carboxymethylcellulose, cross-linked croscarmellose, cross-linked starch, cross-linked polymer, cross-linked polyvinylpyrrolidone, sodium alginate, clay, and gum; and
at least one binder selected from the group consisting of hydroxypropyl cellulose and polyvinylpyrrolidone; sodium laurel sulfate; silica; and magnesium stearate;
   wherein the pharmaceutical formulation is chosen from tablet or capsule fill and Compound 1 is present in an amount ranging from about 5% to about 15% by weight of the tablet or capsule fill.

Embodiment 34: A pharmaceutical formulation comprising Compound 1 and at least one pharmaceutically acceptable excipient, wherein the formulation is in a form chosen from tablet, stock granulation, and capsule forms; wherein Compound 1 is present in an amount to provide a daily dose ranging from about 5 to about 60 mg per day in single or divided doses or multiple doses, wherein Compound 1 is present in an amount ranging from 5% to 50% by weight of tablet, stock granulation, or capsule fill; and further wherein the at least one pharmaceutically acceptable excipient comprises:
at least one diluent comprising at least one of anhydrous lactose, mannitol, and lactose monohydrate in a total amount of diluent ranging from 45% to 90% by weight of tablet or capsule fill;
at least one disintegrating agent comprising sodium starch glycolate in a total amount of disintegrant ranging from 2% to 10% by weight of tablet or capsule fill;
at least one glidant comprising at least one of silicon dioxide and colloidal anhydrous silica in a total amount of glidant ranging from 0.1% to 2% by weight of tablet or capsule fill; and
at least one lubricant comprising magnesium stearate in a total amount of lubricant ranging from 0.1% to 2% by weight of tablet or capsule fill.

Embodiment 35: A blister pack containing the tablet of any one of embodiments 1-32 or the pharmaceutical formulation of embodiments 33 or 34.

Embodiment 36: A bottle containing the tablet of any one of embodiments 1-32 or the pharmaceutical formulation of embodiments 33 or 34.

Embodiment 37: A liquid suspension comprising a crushed tablet of any one of embodiments 1-32 or the dissolved contents of the pharmaceutical formulation of embodiments 33 or 34.

Embodiment 38: A liquid suspension comprising the tablet of any one of embodiments 1-32, wherein the liquid suspension is prepared by crushing the tablet and adding the crushed tablet to water.

Embodiment 39: A method of treating a disease or condition mediated by RIPK1 in a patient in need thereof, comprising administering to the patient the tablet of any one of embodiments 1-32, the pharmaceutical formulation of embodiments 33 or 34, or the liquid suspension of embodiments 37 or 38.

Embodiment 40: Use of the tablet of any one of embodiments 1-32, pharmaceutical formulation of embodiments 33 or 34, or the liquid suspension of embodiments 37 or 38 for treating a disease involving mediation of the RIPK1 receptor.

Embodiment 41: A tablet comprising 4-(3,3-difluoro-2,2-dimethyl-propanoyl)-3,5-dihydro-2H-pyrido[3,4-f][1,4]oxazepine-9-carbonitrile (Compound 1) and at least one pharmaceutically acceptable excipient, wherein the tablet has a hardness of 60 N to 110 N.

Embodiment 42: The tablet of embodiment 41, wherein the tablet has a hardness of 85 N.

Embodiment 43: The tablet of embodiments 41 or 42, wherein Compound 1 is present in an amount to provide a daily dose ranging from about 5 to about 60 mg per day in single or divided doses or multiple doses.

Embodiment 44: The tablet of any one of embodiments 41 to 43, wherein Compound 1 is present in an amount ranging from 5% to 50% by weight of the tablet.

Embodiment 45: The tablet of any one of embodiments 41 to 44, wherein the tablet comprises an intragranular core and an extragranular portion.

Embodiment 46: The tablet of embodiment 45, wherein the intragranular core comprises at least one diluent, at least one disintegrant, at least one glidant, and at least one lubricant.

Embodiment 47: The tablet of embodiments 45 or 46, wherein the extragranular portion comprises at least one disintegrant and at least one lubricant.

Embodiment 48: The tablet of any one of embodiments 41 to 47, wherein Compound 1 is present in at least 50% crystalline form, such as at least 60%, at least 70%, at least 80%, at least 90%, at least 95%, at least 96%, at least 97%, at least 98%, at least 99%, 99.5%, or 100% crystalline.

Embodiment 49: The tablet of embodiment 48, wherein the crystalline form is Form A.

Embodiment 50: The tablet of any one of embodiments 41 to 47, wherein Compound 1 is present in an amorphous form.

Embodiment 51: The tablet of any one of embodiments 41 to 49, wherein Compound 1 is present in crystalline Form A.

Embodiment 52: The tablet of any one of embodiments 41 to 51, wherein the tablet comprises less than 0.1% of a compound having the structure:

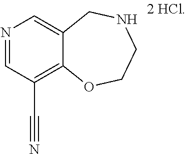 [SMILES: N#Cc1cncc2c1OCCNC2]

Embodiment 53: The tablet of any one of embodiments 41 to 51, wherein the tablet comprises less than 0.1% of a compound having the structure:

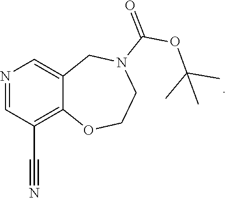 [SMILES: CC(C)(C)OC(=O)N1CCOc2c(C#N)cncc2C1]

Embodiment 54: The tablet of any one of embodiments 41 to 51, wherein the tablet comprises less than 0.1% of a compound having the structure:

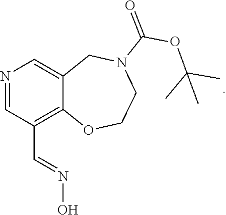 [SMILES: CC(C)(C)OC(=O)N1CCOc2c(/C=N/O)cncc2C1]

Embodiment 55: A blister pack containing the tablet of any one of embodiments 41 to 54.

Embodiment 56: A bottle containing the tablet of any one of embodiments 41 to 54.

Embodiment 57: A liquid suspension comprising a crushed tablet of any one of embodiments 41 to 54.

Embodiment 58: A method of treating a disease or condition mediated by RIPK1 in a patient in need thereof, comprising administering to the patient the tablet of any one of embodiments 41 to 54 or the liquid suspension of embodiment 57.

Embodiment 59: Use of the tablet of any one of embodiments 41 to 54 or the liquid suspension of embodiment 57 for treating a disease involving mediation of the RIPK1 receptor.

Embodiment 60: Tablet of any one of embodiments 1 to 32 or 41 to 54 for use in treating a disease involving mediation of the RIPK1 receptor.

Embodiment 61: Pharmaceutical composition of any one of embodiments 33 to 34 for use in treating a disease involving mediation of the RIPK1 receptor.

Embodiment 62: Pharmaceutical composition of any one of embodiments 33 to 34 for use in treating ALS.

Embodiment 63: Tablet of any one of embodiments 1 to 32 or 41 to 54 for use in treating ALS.

Embodiment 64: Tablet of any one of embodiments 1 to 32 or 41 to 54 prepared by steps comprising: mixing Compound 1 with at least one excipient to obtain a homogenous blend, compressing the blend into a tablet, and spraying the tablet with a film-coating suspension.

Embodiment 65: A crystalline composition that is substantially pure Form A of 4-(3,3-difluoro-2,2-dimethyl-propanoyl)-3,5-dihydro-2H-pyrido[3,4-f][1,4]oxazepine-9-carbonitrile.

Embodiment 66: A crystalline composition that is substantially pure Form A of 4-(3,3-difluoro-2,2-dimethyl-propanoyl)-3,5-dihydro-2H-pyrido[3,4-f][1,4]oxazepine-9-carbonitrile, wherein the crystalline composition exhibits an XRPD pattern profile which is substantially unchanged after storage for 6 months at standard conditions (around 30° C./65% relative humidity).

Embodiment 67: A crystalline composition comprising: substantially pure Form A of 4-(3,3-difluoro-2,2-dimethyl-propanoyl)-3,5-dihydro-2H-pyrido[3,4-f][1,4]oxazepine-9-carbonitrile, and less than or equal to 0.1 percent by weight of a compound having the structure:

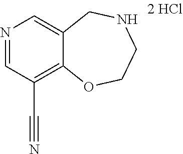 [SMILES: N#Cc1cncc2c1OCCNC2]

Embodiment 68: A crystalline composition comprising: substantially pure Form A of 4-(3,3-difluoro-2,2-dimethyl-propanoyl)-3,5-dihydro-2H-pyrido[3,4-f][1,4]oxazepine-9-carbonitrile, and less than or equal to 0.1 percent by weight of a compound having the structure:

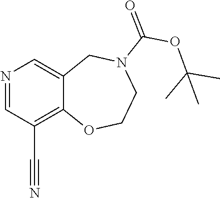 [SMILES: CC(C)(C)OC(=O)N1CCOc2c(C#N)cncc2C1]

Embodiment 69: A crystalline composition comprising: substantially pure Form A of 4-(3,3-difluoro-2,2-dimethyl-propanoyl)-3,5-dihydro-2H-pyrido[3,4-f][1,4]oxazepine-9-carbonitrile, and less than or equal to 0.1 percent by weight of a compound having the structure:

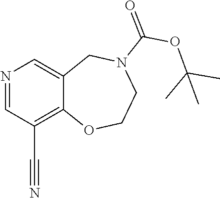 [SMILES: CC(C)(C)OC(=O)N1CCOc2c(C#N)cncc2C1]

Embodiment 70: A crystalline composition comprising at least 97 percent by weight of Form A of 4-(3,3-difluoro-2,2-dimethyl-propanoyl)-3,5-dihydro-2H-pyrido[3,4-f][1,4]oxazepine-9-carbonitrile compared to the total weight of the compound of all forms.

Embodiment 71: A crystalline composition comprising at least 99 percent by weight of Form A of 4-(3,3-difluoro-2,2-dimethyl-propanoyl)-3,5-dihydro-2H-pyrido[3,4-f][1,4]oxazepine-9-carbonitrile compared to the total weight of the compound of all forms.

1.-59. (canceled)

60. A pharmaceutical formulation comprising 4-(3,3-difluoro-2,2-dimethyl-propanoyl)-3,5-dihydro-2H-pyrido[3,4-f][1,4]oxazepine-9-carbonitrile (Compound 1) and at least one pharmaceutically acceptable excipient,
wherein the at least one pharmaceutically acceptable excipient comprises at least one diluent, and
wherein the pharmaceutical formulation comprises a total amount of diluent ranging from about 45% to about 90% by weight of the formulation.

61. The pharmaceutical formulation of claim 60, wherein:
(a) the at least one diluent is chosen from mannitol, lactose monohydrate, anhydrous lactose, microcrystalline cellulose, starch, sorbitol, dextrose, dibasic calcium phosphate, dicalcium phosphate dihydrate, tricalcium phosphate, calcium phosphate, pregelatinized starch, compressible sugar, hydroxypropyl-methylcellulose, hydroxypropylmethylcellulose acetate stearate, sucrose-based diluents, confectioner's sugar, monobasic calcium sulfate monohydrate, calcium sulfate dihydrate, calcium lactate trihydrate, a dextrate, hydrolyzed cereal solids, amylose, powdered cellulose, calcium carbonate, glycine, kaolin, sodium chloride, inositol, and bentonite; or
(b) the at least one diluent is present in a total amount of diluent ranging from about 75% to about 90% by weight of the formulation; or
(c) the at least one diluent is present in a total amount of diluent ranging from about 75% to about 90% by weight of the formulation and comprises at least one of mannitol, lactose monohydrate, anhydrous lactose, microcrystalline cellulose, starch, sorbitol, dextrose, dibasic calcium phosphate, dicalcium phosphate dihydrate, tricalcium phosphate, calcium phosphate, pregelatinized starch, compressible sugar, hydroxypropyl-methylcellulose, hydroxypropylmethylcellulose acetate stearate, sucrose-based diluents, confectioner's sugar, monobasic calcium sulfate monohydrate, calcium sulfate dihydrate, calcium lactate trihydrate, a dextrate, hydrolyzed cereal solids, amylose, powdered cellulose, calcium carbonate, glycine, kaolin, sodium chloride, inositol, and bentonite.

62. The pharmaceutical formulation of claim 60, wherein the at least one pharmaceutically acceptable excipient further comprises at least one glidant, and wherein:
(a) the at least one glidant is chosen from colloidal anhydrous silica, and colloidal silicon dioxide; or
(b) the at least one glidant is present in a total amount of glidant ranging from about 0.1 to about 5% by weight of the formulation; or
(c) the at least one glidant is present in a total amount of glidant ranging from about 0.1 to about 5% by weight of the formulation and comprises at least one of colloidal anhydrous silica and colloidal silicon dioxide.

63. The pharmaceutical formulation of claim 60, wherein the at least one pharmaceutically acceptable excipient further comprises at least one disintegrant, and wherein:
(a) the at least one disintegrant is chosen from sodium starch glycolate, croscarmellose sodium, corn starch, potato starch, pregelatinized starch, methylcrystalline cellulose, methylcellulose, croscarmellose, cross-linked sodium carboxymethyl-cellulose, cross-linked carboxymethylcellulose, cross-linked croscarmellose, crosspovidone, cross-linked polyvinylpyrrolidone, alginic acid, sodium alginate, magnesium aluminum silicate, agar, guar, locust bean, Karaya, pectin, tragacanth, bentonite, citrus pulp, and sodium lauryl sulfate; or
(b) the at least one disintegrant is present in a total amount of disintegrant ranging from about 1 to about 15% by weight of the formulation; or
(c) the at least one disintegrant is present in a total amount of disintegrant ranging from about 1 to about 15% by weight of the formulation and comprises at least one of sodium starch glycolate, croscarmellose sodium, corn starch, potato starch, pregelatinized starch, methylcrystalline cellulose, methylcellulose, croscarmellose, cross-linked sodium carboxymethyl-cellulose, cross-linked carboxymethylcellulose, cross-linked croscarmellose, crosspovidone, cross-linked polyvinylpyrrolidone, alginic acid, sodium alginate, magnesium aluminum silicate, agar, guar, locust bean, Karaya, pectin, tragacanth, bentonite, citrus pulp, and sodium lauryl sulfate.

64. The pharmaceutical formulation of claim 60, wherein the at least one pharmaceutically acceptable excipient further comprises at least one lubricant, and wherein:
(a) the at least one lubricant is chosen from magnesium stearate, stearic acid, calcium hydroxide, talc, sodium stearyl fumerate, mineral oil, hydrogenated soybean oil, aluminum, calcium, magnesium, zinc, sodium stearate, glycerol, talc, wax, boric acid, sodium benzoate, sodium acetate, sodium chloride, leucine, polyethylene glycol, methoxypolyethylene glycol, sodium oleate, sodium benzoate, glyceryl behenate, magnesium lauryl sulfate, sodium lauryl sulfate, colloidal silica, corn starch, silicone oil, and surfactant; or
(b) the at least one lubricant is present in a total amount of lubricant ranging from about 0.1 to about 5% by weight of the formulation; or
(c) the at least one lubricant is present in a total amount of lubricant ranging from about 0.1 to about 5% by weight of the formulation and comprises at least one of magnesium stearate, stearic acid, calcium hydroxide, talc, sodium stearyl fumerate, mineral oil, hydrogenated soybean oil, aluminum, calcium, magnesium, zinc, sodium stearate, glycerol, talc, wax, boric acid, sodium benzoate, sodium acetate, sodium chloride, leucine, polyethylene glycol, methoxypolyethylene glycol, sodium oleate, sodium benzoate, glyceryl behenate, magnesium lauryl sulfate, sodium lauryl sulfate, colloidal silica, corn starch, silicone oil, and surfactant.

65. The pharmaceutical formulation of claim 60, wherein Compound 1 is present in a form that is at least about 50% crystalline.

66. The pharmaceutical formulation of claim 65, wherein the crystalline form is Form A.

67. The pharmaceutical formulation of claim 60, wherein Compound 1 is present in an amorphous form.

68. The pharmaceutical formulation of claim 60, wherein Compound 1 is present in an amount ranging from about 5 mg to about 60 mg.

69. The pharmaceutical formulation of claim 68, wherein Compound 1 is present in an amount of about 20 mg.

70. A method of treating a disease or condition mediated by RIPK1 in a patient in need thereof, comprising administering to the patient the pharmaceutical formulation of claim 60.

71. A pharmaceutical formulation comprising 4-(3,3-difluoro-2,2-dimethyl-propanoyl)-3,5-dihydro-2H-pyrido[3,4-f][1,4]oxazepine-9-carbonitrile (Compound 1) and at least one diluent, wherein the total amount of diluent comprises from about 45% to about 90% by weight of the formulation, and wherein the formulation comprises a tablet having a hardness of 60 N to 110 N.

72. The pharmaceutical formulation of claim 71, wherein the tablet has a hardness of 85 N.

73. The pharmaceutical formulation of claim 71, wherein the at least one diluent is selected from the group consisting of lactose, sucrose, dextrose, dextrates, maltodextrin, mannitol, xylitol, sorbitol, cyclodextrins, calcium phosphate, calcium sulfate, starches, modified starches, cellulose, microcrystalline cellulose, microcellulose, and talc.

74. The pharmaceutical formulation of claim 71, further comprising:
at least one disintegrating agent selected from the group consisting of natural starch, a pregelatinized starch, sodium starch, methylcrystalline cellulose, methylcellulose, croscarmellose, croscarmellose sodium, cross-linked sodium carboxymethylcellulose, cross-linked carboxymethylcellulose, cross-linked croscarmellose, cross-linked starch, cross-linked polymer, cross-linked polyvinylpyrrolidone, sodium alginate, clay, and gum; and
at least one binder selected from the group consisting of hydroxypropyl cellulose and polyvinylpyrrolidone; sodium laurel sulfate; silica; and magnesium stearate;
wherein the pharmaceutical formulation is chosen from tablet or capsule fill and Compound 1 is present in an amount ranging from about 5% to about 15% by weight of the tablet or capsule fill.

75. A method of treating a disease or condition mediated by RIPK1 in a patient in need thereof, comprising administering to the patient the pharmaceutical formulation of claim 71.

76. A process for preparing a pharmaceutical formulation of 4-(3,3-difluoro-2,2-dimethyl-propanoyl)-3,5-dihydro-2H-pyrido[3,4-f][1,4]oxazepine-9-carbonitrile (Compound 1), the process comprising the steps of:
mixing Compound 1 with at least one excipient to obtain a homogenous blend,
compressing the blend, and
spraying the compressed blend with a suspension.

77. The process of claim 76, wherein the suspension is a film-coating suspension.

78. The process of claim 76, further comprising one or more of the following steps: sieving and dry roller compaction.

79. A pharmaceutical formulation comprising 4-(3,3-difluoro-2,2-dimethyl-propanoyl)-3,5-dihydro-2H-pyrido[3,4-f][1,4]oxazepine-9-carbonitrile (Compound 1) and at least one pharmaceutically acceptable excipient, the pharmaceutical formulation prepared by the process of claim 76, wherein the formulation comprises a tablet having a hardness of 60 N to 110 N.
